SEQUENCE LISTING (31 sequences)

SEQ ID NO: 1 - protein, 159 aa, Homo sapiens
KLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 2 - protein, 317 aa, Homo sapiens
  misc_feature (279)..(279): Xaa can be any naturally occurring amino acid
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGXFFKLRSGEEISIEVSNPSLLDPDQDATYFGAFKVRDID

SEQ ID NO: 3 - protein, 159 aa, Homo sapiens
  misc_feature (121)..(121): Xaa can be any naturally occurring amino acid
KLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGXFFKLRSGEEISIEVSNPSLLDPDQDATYFGAFKVRDID

SEQ ID NO: 4 - protein, 12 aa, Artificial
  RANKL fragment
HFYSINVGGFFK

SEQ ID NO: 5 - protein, 12 aa, Artificial
  RANKL fragment
HFYSINVGRFFK

SEQ ID NO: 6 - protein, 15 aa, Artificial
  mouse/human RANKL fragment
EFHFYSINVGGFFKL

SEQ ID NO: 7 - protein, 15 aa, Artificial
  human TNF fragment
KPWYEPIYLGGVFQL

SEQ ID NO: 8 - protein, 15 aa, Artificial
  human CD40L fragment
PCGQQSIHLGGVFEL

SEQ ID NO: 9 - protein, 15 aa, Artificial
  human TRAIL fragment
EYGLYSIYQGGIFEL

SEQ ID NO: 10 - protein, 14 aa, Artificial
  human BAFFfragment
LPNNSCYSAGIAKL

SEQ ID NO: 11 - protein, 15 aa, Artificial
  human APRIL fragment
DRAYNSCYSAGVFHL

SEQ ID NO: 12 - protein, 15 aa, Artificial
  human LTa fragment
EPWLHSMYHGAAFQL

SEQ ID NO: 13 - protein, 250 aa, Homo sapiens [sequence omitted]

SEQ ID NO: 14 - protein, 330 aa, Homo sapiens [sequence omitted]

SEQ ID NO: 15 - protein, 389 aa, Homo sapiens
MGYPEVERRELLPAAAPRERGSQGCGCGGAPARAGEGNSCLLFLGFFGLSLALHLLTLCCYLELRSELRRERGAESRLGGSGTPGTSGTLSSLGGLDPDSPITSHLGQPSPKQQPLEPGEAALHSDSQDGHQMALLNFFFPDEKPYSEEESRRVRRNKRSKSNEGADGPVKNKKKGKKAGPPGPNGPPGPPGPPGPQGPPGIPGIPGIPGTTVMGPPGPPGPPGPQGPPGLQGPSGAADKAGTRENQPAVVHLQGQGSAIQVKNDLSGGVLNDWSRITMNPKVFKLHPRSGELEVLVDGTYFIYSQVYYINFTDFASYEVVVDEKPFLQCTRSIETGKTNYNTCYTAGVCLLKARQKIAVKMVHADISINMSKHTTFFGAIRLGEAPAS

SEQ ID NO: 16 - protein, 391 aa, Homo sapiens
MGYPEVERRELLPAAAPRERGSQGCGCGGAPARAGEGNSCLLFLGFFGLSLALHLLTLCCYLELRSELRRERGAESRLGGSGTPGTSGTLSSLGGLDPDSPITSHLGQPSPKQQPLEPGEAALHSDSQDGHQMALLNFFFPDEKPYSEEESRRVRRNKRSKSNEGADGPVKNKKKGKKAGPPGPNGPPGPPGPPGPQGPPGIPGIPGIPGTTVMGPPGPPGPPGPQGPPGLQGPSGAADKAGTRENQPAVVHLQGQGSAIQVKNDLSGGVLNDWSRITMNPKVFKLHPRSGELEVLVDGTYFIYSQVEVYYINFTDFASYEVVVDEKPFLQCTRSIETGKTNYNTCYTAGVCLLKARQKIAVKMVHADISINMSKHTTFFGAIRLGEAPAS

SEQ ID NO: 17 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 18 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 19 - protein, 205 aa, Homo sapiens
MTPPERLFLPRVCGTTLHLLLLGLLLVLLPGAQGLPGVGLTPSAAQTARQHPKMHLAHSNLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 20 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 21 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 22 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 23 - protein, 244 aa, Homo sapiens
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 24 - protein, 281 aa, Homo sapiens [sequence omitted]

SEQ ID NO: 25 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 26 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 27 - protein, 199 aa, Homo sapiens
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 28 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 29 - protein, 208 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGLFGFWNWGLKVKCFLRHLIWTAHCFIPLTQLVFMQALQSWRNHHCSHFTDEENRGVNR

SEQ ID NO: 30 - protein, 234 aa, Homo sapiens
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 31 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

(19) United States
(12) Patent Application Publication
(10) Pub. No.: US 2014/0165223 A1
(43) Pub. Date: Jun. 12, 2014

(54) TNF SUPERFAMILY TRIMERIZATION INHIBITORS

(71) Applicant: B.S.R.C. "Alexander Fleming", Varkiza (GR)

(72) Inventors: Eleni Ntouni, Spata (GR); Georgios Kollias, Aghia Paraskevi (GR)

(73) Assignee: B.S.R.C. "Alexander Fleming", Varkiza (GR)

(21) Appl. No.: 14/178,469

(22) Filed: Feb. 12, 2014

Related U.S. Application Data
(63) Continuation of application No. PCT/EP2012/065716, filed on Aug. 10, 2012.
(60) Provisional application No. 61/522,728, filed on Aug. 12, 2011.

(51) Int. Cl. C07K 14/47; A61K 31/4178; A61K 31/496; A61K 31/4045; A61K 31/166; A61K 38/17

(52) U.S. Cl. CPC C07K 14/47; A61K 31/166; A61K 38/1709; A61K 31/496; A61K 31/4045; A61K 31/4178
USPC 800/18; 514/616; 564/155; 530/350; 536/23.5; 514/16.6; 424/450; 514/16.7; 514/16.9; 514/254.09; 544/373; 514/419; 548/492; 514/397; 548/312.1; 514/414; 548/454; 435/375; 435/320.1; 435/254.2; 435/252.3; 435/254.11; 435/348; 435/325; 435/254.21; 435/252.33; 435/252.31; 435/254.4; 435/352; 435/358; 435/365.1; 435/254.23

(57) ABSTRACT

Described are methods and compositions for inhibiting the trimerization of ligands belonging to the TNF superfamily, in particular, inhibiting RANKL trimerization. Accordingly, the methods and compositions provided herein can be used to treat disorders associated with increased RANK signaling, in particular those related to bone loss. Compounds that inhibit trimerization of ligands belonging to the TNF superfamily are also described.

### CROSS-REFERENCE TO RELATED APPLICATIONS

This application is a continuation of co-pending International Patent Application PCT/EP2012/065716, filed Aug. 10, 2012, designating the United States of America and published in English as International Patent Publication WO 2013/024040 A2 on Feb. 21, 2013, which claims the benefit under Article 8 of the Patent Cooperation Treaty and under 35 U.S.C. §119(e) to U.S. Provisional Patent Application Ser. No. 61/522,728, filed Aug. 12, 2011, the disclosure of each of which is hereby incorporated herein in its entirety by this reference.

### STATEMENT ACCORDING TO 37 C.F.R. §1.821(c) or (e)

### Sequence Listing Submitted as a TXT and PDF Files

Pursuant to 37 C.F.R. §1.821(c) or (e), files containing a TXT version and a PDF version of the Sequence Listing have been submitted concomitant with this application, the contents of which are hereby incorporated by reference.

### TECHNICAL FIELD

This disclosure relates to methods and compositions for inhibiting the trimerization of ligands belonging to the TNF superfamily. In particular, the disclosure relates to inhibiting RANKL trimerization. Accordingly, the methods and compositions provided herein can be used to treat disorders associated with increased RANK signaling, in particular those related to bone loss.

### BACKGROUND

Bone remodeling is a constant process through the synthesis of bone matrix by osteoblasts and the coordinate bone resorption by osteoclasts.^{[1, 2]} Normally, osteoblastic and osteoclastic activities are balanced so that skeletal integrity is preserved. Perturbations in bone remodeling can result in skeletal abnormalities, such as osteopetrosis and osteoporosis, which are characterized by excessive or decreased bone mass, due to impaired or enhanced osteoclast activity. RANKL is the primary mediator of osteoclast-induced bone resorption^{[3]} and belongs to the TNF superfamily^{[4, 5]} that is characterized by homotrimerization. It is a type II transmembrane protein that consists of a short N-terminal cytoplasmic domain and a conserved extracellular domain forming an antiparallel β-sheet that is predicted to assemble into a trimer required for receptor activation.^{[6, 7]} Soluble RANKL is generated either by proteolytic processing of the transmembrane form or by alternative splicing.^{[8, 9]} RANKL is expressed on activated T lymphocytes^{[4, 5]} as well as on stromal cells^{[10, 11]} and binds as a trimer to its receptor RANK that is expressed on the surface of osteoclast precursors and mature osteoclasts. This interaction is necessary for osteoclast differentiation, activity and survival,^{[10, 12]} which subsequently lead to bone resorption. Osteoprotegerin (OPG), a decoy receptor of RANKL, inhibits the binding of RANKL to RANK and thereby limits osteoclastogenesis.^{[11]} Genetic ablations of both RANKL^{[13, 14]} and RANK^{[15, 16]} result in severe osteopetrosis due to complete lack of osteoclast formation demonstrating that RANKL and RANK are indispensable for osteoclastogenesis. Absence of OPG causes increased osteoclastogenesis and osteopenia.^{[17]} While RANKL is best known for its role in bone resorption, it also plays multiple roles in immune system,^{[4, 5, 13, 18, 19]} mammary gland development during pregnancy,^{[20]} thermoregulation,^{[21]} cancer metastasis,^{[22]} and hormone-derived breast development.^{[23]}

As a result of its effects on the skeleton, RANKL is a major therapeutic target for the suppression of bone resorption in osteoporosis, rheumatoid arthritis and cancer metastasis.^{[24]} Indeed, clinical trials with denosumab, a fully human monoclonal antibody against RANKL, showed an increased bone mass and reduced incidence of fractures in postmenopausal women with osteoporosis^{[25]} and in prostate cancer patients receiving androgen-deprivation therapy.^{[26]} This antibody has been recently approved in the USA and EU for the treatment of patients with osteoporosis and in prostate cancer patients undergoing hormonal ablation therapy. On the other hand, a variety of mutations localized within the extracellular domain of RANKL have been recently reported in children with autosomal recessive osteopetrosis (ARO) (OMIM 602642), an incurable rare genetic disease.^{[27]} However, animal models bearing functional mutations in the Rankl gene have not been reported yet, hampering not only the identification of critical residues involved in RANKL function but also the elucidation of the molecular pathogenic mechanisms underlying ARO.

### DISCLOSURE

Provided is a method for inhibiting trimerization of a TNF superfamily member polypeptide comprising contacting the polypeptide with a trimerization inhibitor selected from
a) a dominant negative TNF superfamily member polypeptide or fragment thereof, preferably having a dominant negative mutation in the trimerization domain,
b) a compound that binds to the TNF superfamily member polypeptide in the F beta-strand of the polypeptide, preferably at the glycine residue that corresponds to position 279 in human RANKL, provided that when the trimerization inhibitor is 6,7-Dimethyl-3-[[methyl[2-[methyl[[1-[3-(trifluoromethyl)phenyl]-1H-indol-3-yl]methyl]amino]ethyl]amino]methyl]-(4H-1-Benzopyran-4-one), the TNF superfamily member polypeptide is not TNF-alpha.

Preferably, when the trimerization inhibitor is a compound of formula 1 as described herein, or a stereoisomer thereof, tautomer thereof, or mixture thereof in any ratio; a pharmaceutically acceptable salt, pharmaceutically acceptable solvate, or pharmaceutically acceptable polymorph thereof; the TNF superfamily member polypeptide is not TNF-alpha. More preferably, when the trimerization inhibitor is a compound that binds to the TNF superfamily member polypeptide in the F beta-strand, the TNF superfamily member polypeptide is not TNF-alpha. Preferably, the TNF superfamily member polypeptide or fragment thereof comprises a mutation in the F beta-strand, preferably in the glycine residue that corresponds to position 279 in human RANKL. In some embodiments, the method is an in vitro method.

Preferably, a method is provided for inhibiting trimerization of a TNF superfamily member polypeptide comprising contacting the polypeptide with T23 or a functional derivative thereof, or a functional derivative of 6,7-Dimethyl-3-[[methyl[2-[methyl[[1-[3-(trifluoromethyl)phenyl]-1H-indol-3-yl]methyl]amino]ethyl]amino]methyl]-(4H-1-Benzopyran-4-one); preferably selected from PRA123, PRA224, PRA333, PRA738, and PRA828, most preferably PRA224.

Preferably, a method is provided for inhibiting TNF-induced cell death comprising contacting a cell susceptible of TNF-induced cell death with T23 or a functional derivative thereof, or a functional derivative of 6,7-Dimethyl-3-[[methyl[2-[methyl[[1-[3-(trifluoromethyl)phenyl]-1H-indol-3-yl]methyl]amino]ethyl]amino]methyl]-(4H-1-Benzopyran-4-one); preferably selected from PRA123, PRA224, PRA333, PRA738, and PRA828, most preferably PRA224. In some embodiments, the method is an in vitro method. In some embodiments, the cell is a non-human cell.

Preferably, a method is provided for reducing TNF-induced matrix metalloproteinase release comprising contacting a cell susceptible of TNF-induced matrix metalloproteinase release with T23 or a functional derivative thereof, or a functional derivative of 6,7-Dimethyl-3-[[methyl[2-[methyl[[1-[3-(trifluoromethyl)phenyl]-1H-indol-3-yl]methyl]amino]ethyl]amino]methyl]-(4H-1-Benzopyran-4-one); preferably selected from PRA123, PRA224, PRA333, PRA738, and PRA828, most preferably PRA224. Preferably, the cell is a synovial fibroblast. In some embodiments, the method is an in vitro method. In some embodiments, the cell is a non-human cell.

Also provided is a method for inhibiting osteoclast formation or decreasing bone loss in an individual, the method comprising administering to an individual in need thereof a therapeutically effective amount of a compound that inhibits trimerization of RANKL selected from
a) a dominant negative RANKL polypeptide or fragment thereof, preferably having a dominant negative mutation in the trimerization domain,
b) a compound that binds to the TNF superfamily member polypeptide in the F beta-strand of the polypeptide, preferably at the glycine residue that corresponds to position 279 in human RANKL.

Preferably, the RANKL polypeptide or fragment thereof comprises a mutation in the F beta-strand, preferably at the glycine residue that corresponds to position 279 in human RANKL. Preferably, the compound is T23 or a functional derivative thereof, or a functional derivative of 6,7-Dimethyl-3-[[methyl[2-[methyl[[1-[3-(trifluoromethyl)phenyl]-1H-indol-3-yl]methyl]amino]ethyl]amino]methyl]-(4H-1-Benzopyran-4-one); preferably selected from PRA123, PRA224, PRA333, PRA738, and PRA828, most preferably PRA224.

Further provided is a method for preventing, treating, or reducing symptoms in an individual afflicted with osteoporosis, rheumatoid arthritis, multiple myeloma, bone metastasis, juvenile osteoporosis, osteogenesis imperfecta, hypercalcemia, hyperparathyroidism, osteomalacia, osteohalisteresis, osteolytic bone disease, osteonecrosis, Paget's disease of bone, bone loss due to rheumatoid arthritis, inflammatory arthritis, osteomyelitis, periodontal bone loss, bone loss due to cancer, age-related loss of bone mass, osteopenia, and inflammatory bowel syndrome, comprising administering to an individual in need thereof a therapeutically effective amount of a compound that inhibits trimerization of RANKL selected from
a) a dominant negative RANKL polypeptide or fragment thereof, preferably having a dominant negative mutation in the trimerization domain,
b) a compound that binds to the TNF superfamily member polypeptide in the F beta-strand of the polypeptide, preferably at the glycine residue that corresponds to position 279 in human RANKL.

Preferably, the RANKL polypeptide or fragment thereof comprises a mutation in the F beta-strand, preferably at the glycine residue that corresponds to position 279 in human RANKL. Preferably, the compound is T23 or a functional derivative thereof, or a functional derivative of 6,7-Dimethyl-3-[[methyl[2-[methyl[[1-[3-(trifluoromethyl)phenyl]-1H-indol-3-yl]methyl]amino]ethyl]amino]methyl]-(4H-1-Benzopyran-4-one); preferably selected from PRA123, PRA224, PRA333, PRA738, and PRA828, most preferably PRA224.

The compound that binds to the TNF superfamily member polypeptide may be a compound as depicted in FIG. 23 or a stereoisomer thereof, tautomer thereof, or mixture thereof in any ratio; a pharmaceutically acceptable salt, pharmaceutically acceptable solvate, or pharmaceutically acceptable polymorph thereof. Preferably, the compound is compound 1 of FIG. 23 (T23).

In certain embodiments, the compound that binds to the TNF superfamily member polypeptide is a compound of formula 1, or a stereoisomer thereof, tautomer thereof, or mixture thereof in any ratio; a pharmaceutically acceptable salt, pharmaceutically acceptable solvate, or pharmaceutically acceptable polymorph thereof;

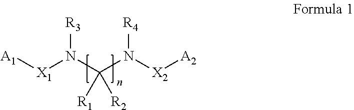

wherein:
A₁ and A₂ are independently a substituted or unsubstituted heterocyclic system selected from:

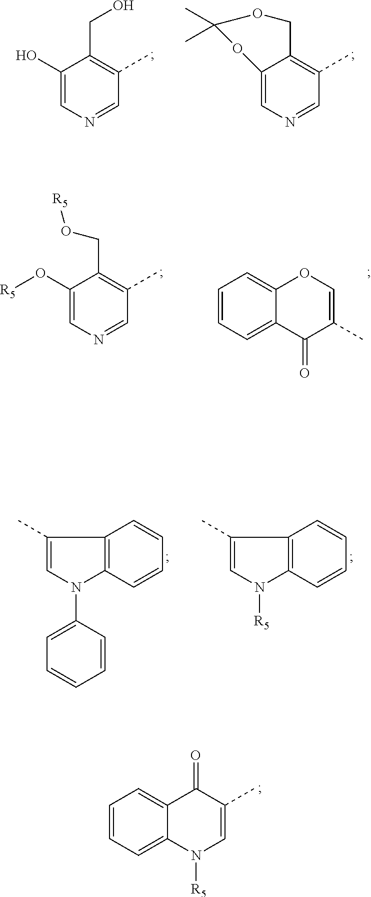

wherein the dotted line indicates the point of attachment, R₅ is hydrogen or (C₁-C₄)-alkyl group and the rings of the heterocyclic systems herein above may be substituted with groups selected from (C₁-C₄)-alkyl, (C₁-C₄)-alkoxy, hydroxyl, hydroxy-(C₁-C₄)-alkyl (e.g., hydroxymethyl or 1-hydroxyethyl or 2-hydroxyethyl), and fluoroalkyl (e.g., CF₃);
X₁ and X₂ are independently a carbonyl group or a methylene (—CH₂—) group; n is an integer from 2-4;
R₁ and R₂ are independently, hydrogen or (C₁-C₄)-alkyl group;
R₃ and R₄ are independently, hydrogen or (C₁-C₄)-alkyl group; and
R₃ and R₄ can optionally form a ring system; with a proviso that when A₁ and A₂ are 1-(3-(thfluoromethyl)phenyl)-1H-indole and 6,7-dimethyl-4H-chromen-4-one respectively and X₁ and X2 are independently a methylene (—CH₂—) group, R₃ and R₄ form a ring system, preferably, wherein the compound is 6,7-Dimethyl-3-[[methyl[2-[methyl[[1-[3-(trifluoromethyl)phenyl]-1H-indol-3-yl]methyl]amino]ethyl]amino]methyl]-(4H-1-Benzopyran-4-one), also known as SPD304.

In certain embodiments, the compound that binds to the TNF superfamily member polypeptide is a compound of formula 1, or a stereoisomer thereof, tautomer thereof, or mixture thereof in any ratio; a pharmaceutically acceptable salt, pharmaceutically acceptable solvate, or pharmaceutically acceptable polymorph thereof;

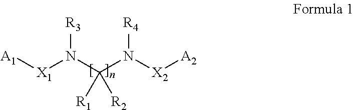

wherein:
A₁ and A₂ are independently a substituted or unsubstituted heterocyclic system selected from:

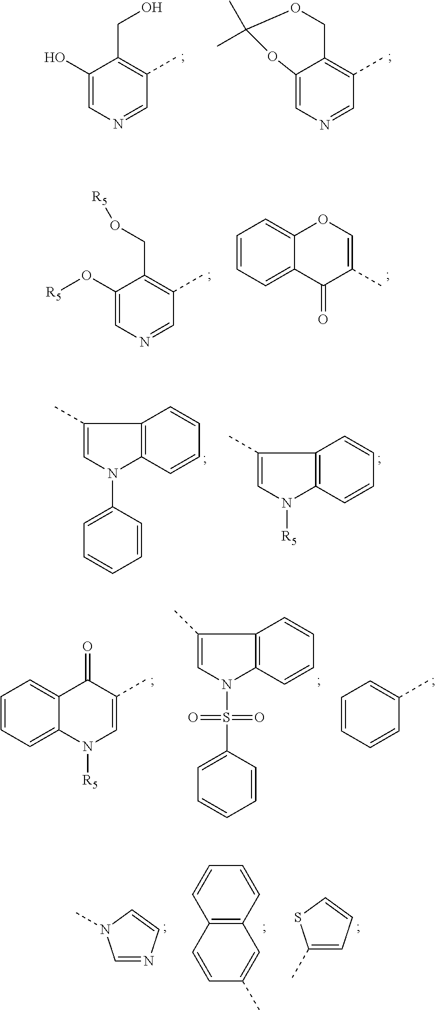

wherein the dotted line indicates the point of attachment, R₅ is hydrogen or (C₁-C₄)-alkyl group and the rings of the heterocyclic systems herein above are unsubstituted or substituted with one or more groups selected from (C₁-C₄)-alkyl, (C₁-C₄)-alkoxy, hydroxyl, hydroxy-(C₁-C₄)-alkyl (e.g., hydroxymethyl or 1-hydroxyethyl or 2-hydroxyethyl), fluoroalkyl (e.g., CF₃), halide (e.g., fluoro); nitro (NO₂) and amino (NH₂);
X₁ and X₂ are independently a carbonyl group or a methylene (—CH₂—) group; n is an integer from 2-4;
R₁ and R₂ are independently, hydrogen or (C₁-C₄)-alkyl group;
R₃ and R₄ are independently, hydrogen or (C₁-C₄)-alkyl group; or wherein R₃ and R₄ are a single (C₁-C₈)-hydrocarbon group connecting the two nitrogen atoms of formula 1, which group may be selected from saturated hydrocarbon (e.g., C₂H₄) and aromatic hydrocarbon (e.g., phenyl).

Preferably, the rings of the heterocyclic systems are unsubstituted or substituted with one or more groups selected from trifluoromethyl (CF₃), fluoro (F); nitro (NO₂) and amino (NH₂). Preferably, A₁ and A₂ are selected from:

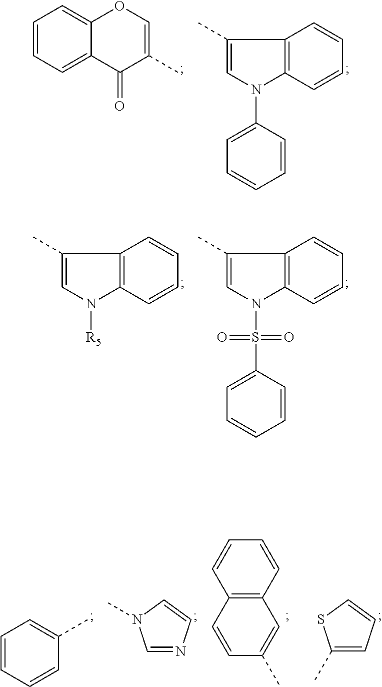

more preferably selected from

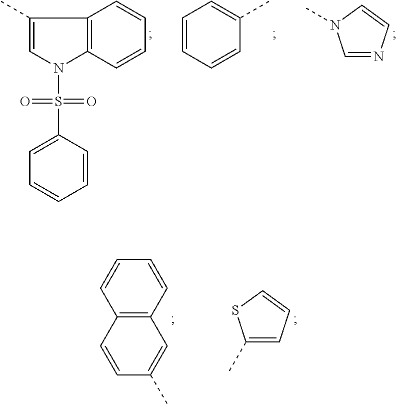

In a further aspect, compounds are provided having Formula 1, or a stereoisomer thereof, tautomer thereof, or mixture thereof in any ratio; a pharmaceutically acceptable salt, pharmaceutically acceptable solvate, or pharmaceutically acceptable polymorph thereof;

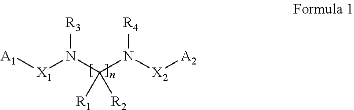

wherein:
A₁ and A₂ are independently a substituted or unsubstituted heterocyclic system selected from:

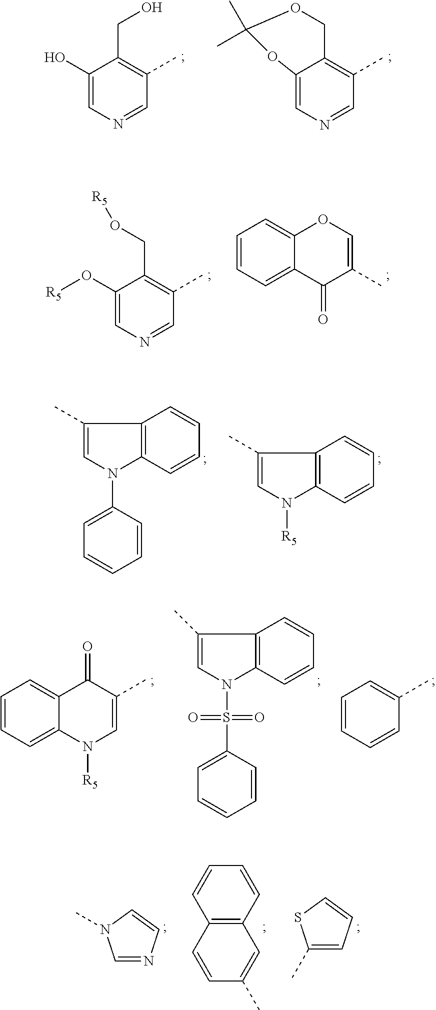

wherein the dotted line indicates the point of attachment, R₅ is hydrogen or (C₁-C₄)-alkyl group and the rings of the heterocyclic systems herein above are unsubstituted or substituted with one or more groups selected from (C₁-C₄)-alkyl, (C₁-C₄)-alkoxy, hydroxyl, hydroxy-(C₁-C₄)-alkyl (e.g., hydroxymethyl or 1-hydroxyethyl or 2-hydroxyethyl), fluoroalkyl (e.g., CF₃), halide (e.g., fluoro); nitro (NO₂) and amino (NH₂);
X₁ and X₂ are independently a carbonyl group or a methylene (—CH₂—) group; n is an integer from 2-4;
R₁ and R₂ are independently, hydrogen or (C₁-C₄)-alkyl group;
R₃ and R₄ are independently, hydrogen or (C₁-C₄)-alkyl group;
or wherein R₃ and R₄ are a single (C₁-C₈)-hydrocarbon group connecting the two nitrogen atoms of formula 1, which group may be selected from saturated hydrocarbon (e.g., C₂H₄) and aromatic hydrocarbon (e.g., phenyl);
with the proviso that when A₁ and A₂ are both selected from:

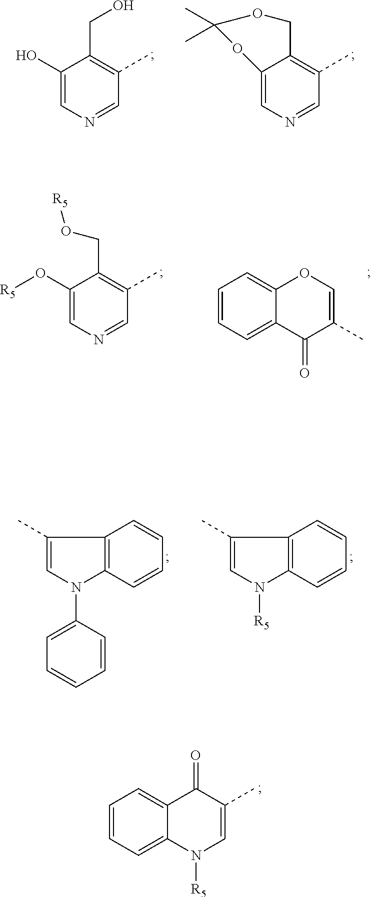

then at least one of the heterocyclic systems is substituted with one or more groups selected from halide (e.g., fluoro); nitro (NO₂) and amino (NH₂).

Preferably, the rings of the heterocyclic systems are unsubstituted or substituted with one or more groups selected from trifluoromethyl (CF₃), fluoro (F); nitro (NO₂) and amino (NH₂). Preferably, A₁ and A₂ are selected from:

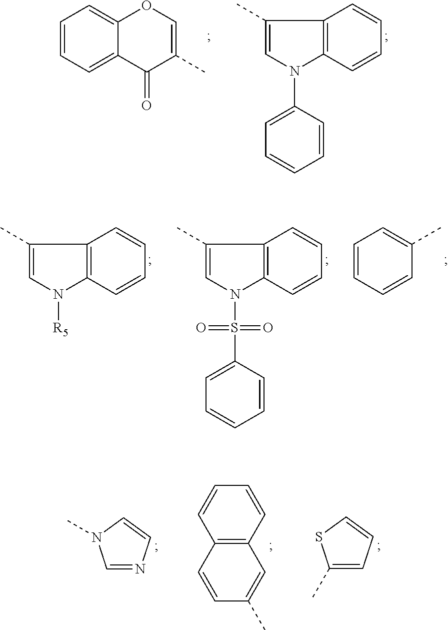

more preferably selected from

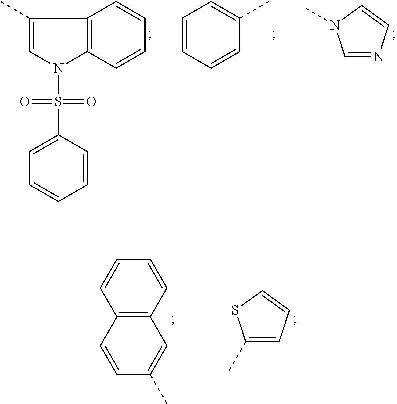

Preferably, a compound is provided selected from the compounds listed in FIG. 22. Preferably, the compound is selected from PRA123, PRA224, PRA333, PRA738, and PRA828; more preferably selected from PRA828, most preferably PRA224. The compounds disclosed above are particularly useful in the methods disclosed herein.

Further provided is a TNF superfamily member polypeptide or fragment thereof that inhibits trimerization of the TNF superfamily member. As used herein, the polypeptide or fragment thereof has a “dominant negative effect.”

Preferably, the polypeptide or fragment thereof has a dominant negative mutation in the trimerization domain, preferably comprising a mutation in F beta-strand, more preferably in the glycine residue that corresponds to position 279 in human RANKL.

In certain embodiments, the TNF superfamily member polypeptide or a functional fragment thereof comprises an amino acid sequence having at least 80% sequence identity to K L E A Q P F A H L T I N A T D I P S G S H K V S L S S W Y H D R G W A K I S N M T F S N G K L I V N Q D G F Y Y L Y A N I C F R H H E T S G D L A T E Y L Q L M V Y V T K T S I K I P S S H T L M K G G S T K Y W S G N S E F H F Y S I N V G X F F K L R S G E E I S I E V S N P S L L D P D Q D A T Y F G A F K V R D I D (SEQ ID NO:3), wherein X is not glycine.

Further provided is a fragment of a wild-type TNF superfamily member polypeptide that has a dominant negative effect. Such a fragment is also useful for the methods of inhibiting trimerization and for treating RANKL related disorders as described herein.

Further provided is an isolated nucleic acid encoding the TNF superfamily member polypeptide or fragment thereof as described herein; a non-human animal comprising the nucleic acid, preferably comprising a nucleic acid encoding for an amino acid sequence having at least 95% identity to SEQ ID NO:2 or SEQ ID NO:3; a vector comprising a nucleic acid as described herein; and a cell comprising the nucleic acid or the cell.

Also provided is a pharmaceutical composition comprising the TNF superfamily member polypeptide or fragment thereof as described herein, compounds of formula I, or T23, and a pharmaceutically acceptable carrier. Also provided is a liposome comprising the TNF superfamily member polypeptide or fragment thereof as described herein. The pharmaceutical compositions and liposomes are particularly useful for treating a bone disorder or a disease having bone disorder as a symptom. Preferred disorders include, osteoporosis, rheumatoid arthritis, multiple myeloma, bone metastasis, juvenile osteoporosis, osteogenesis imperfecta, hypercalcemia, hyperparathyroidism, osteomalacia, osteohalisteresis, osteolytic bone disease, osteonecrosis, Paget's disease of bone, bone loss due to rheumatoid arthritis, inflammatory arthritis, osteomyelitis, periodontal bone loss, bone loss due to cancer, age-related loss of bone mass, osteopenia, and inflammatory bowel syndrome, more preferably postmenopausal associated osteoporosis.

Provided are TNF superfamily trimerization inhibitors for use in the preparation of a medicament for inhibiting osteoclast formation or decreasing bone loss; for preventing, treating, or reducing symptoms in an individual afflicted with osteoporosis, rheumatoid arthritis, multiple myeloma, bone metastasis, juvenile osteoporosis, osteogenesis imperfecta, hypercalcemia, hyperparathyroidism, osteomalacia, osteohalisteresis, osteolytic bone disease, osteonecrosis, Paget's disease of bone, bone loss due to rheumatoid arthritis, inflammatory arthritis, osteomyelitis, periodontal bone loss, bone loss due to cancer, age-related loss of bone mass, osteopenia, and inflammatory bowel syndrome.

### BRIEF DESCRIPTION OF THE DRAWINGS

FIG. 1. Severe osteopetrosis in tles/tles mice. (Panel A) Representative von Kossa-stained proximal tibia sections are shown for 4-week-old WT and tles/tles mice (n=6). (Panel B) Representative serial sections of distal femurs stained with hematoxylin/eosin (H/E) and hematoxylin/TRAP (H/TRAP) (n=6). (Panel C) TRAP staining of osteoclast cultures derived from BM cells or splenocytes (SP) treated with M-CSF and RANKL. (Panel D) TRAP staining of cocultures between BM cells and primary calvarial osteoblasts (OB) in the presence of 1.25 (OH)₂ vitamin D3 and PGE2. Representative data of three experiments performed in triplicate. Bars: (Panel A) 200 μm; (Panels B, C and D) 100 μm.

FIG. 2. Mapping, identification and representation of the ties mutation. (FIG. 2A) Based on genome-wide genetic analysis, the causal mutation was mapped to chromosome 14. (FIG. 2B) DNA sequencing of the Rankl gene in WT control, tles/+ heterozygous and tles/tles homozygous mice revealed that the mutation corresponds to a G to A transition (asterisk) causing a glycine to arginine substitution at residue 278. (FIG. 2C) Ribbon diagram of the RANKL trimer viewed down the three-fold symmetry axis represents a trimer consisting of two WT monomers containing G278 (orange) and one monomer containing the G278R mutated residue (yellow). (FIG. 2D) Space-filling diagram of the RANKL monomer viewed towards the trimer interface with the mutation G278R (yellow chicken wire) in place. Hydrophobic amino acids are colored purple, polar in green and charged (+/−) in blue/red respectively. (FIG. 2E) The sequence of the extracellular F beta-strand of the murine RANKL is aligned to the beta strand of human TNF family cytokines RANKL, TNF, CD40L, TRAIL, BAFF, APRIL, and LTα. The degree of homology correlates with grey scaling, 0-50% conservation (no color), 50-70% (grey), 70-90% (dark grey), >90% (black). Asterisk indicates the residue that corresponds to mouse G278. Sequences appear in FIG. 2E (SEQ ID NO:6; SEQ ID NO:4; SEQ ID NOS:7-12, respectively).

FIG. 3. Genetic confirmation of the RANKL G278R mutation. (FIG. 3A) Serial sections of tibiae from 3-week-old Rankl^{-/tles} compound heterozygous mice were stained with hematoxylin and TRAP (H/TRAP). Bar: 100 μm. (FIG. 3B) Representative femur trabecular areas from Rankl^{+/+}, Rankl^{-/-} and Rankl^{tles/tles} mice scanned with microCT (n=6 per group). (FIG. 3C) Histomorphometric analysis of structural bone parameters of femurs from Rankl^{+/+} (n=12), Rankl^{+/-} (n=6), Rankl^{-/-} (n=6), Rankl^{tles/+} (n=6), and Rankl^{tles/tles} (n=6) littermate mice at 4 weeks of age. BV/TV, bone volume/total volume; NOc/T.Ar, number of osteoclasts/total area; NOc/B.Pm, number of osteoclasts/bone perimeter/mm; Tr.Th, trabecular thickness (mm); Tr.N, trabecular number/mm; Tr.S, trabecular separation/mm3. ***P<0.0001 and **P<0.001 when Rankl^{-/-} and Rankl^{tles/tles} mice were compared to the rest groups. (FIG. 3D) Osteoclast formation is restored by recombinant RANKL administration. Daily subcutaneous injections of recombinant RANKL at 150 μg/kg in Rankl^{tles/tles} mice (n=4) induce formation of TRAP+ cells in trabecular bones. Representative TRAP staining of distal femur sections are shown. Scale bar: 50 μm.

FIG. 4. RANKL^{G278R} fails to trimerize and bind to RANK but interacts with WT RANKL. (FIG. 4A) Recombinant WT GST-RANKL and GST-RANKL^{G278R} were resolved either on native or SDS reduced polyacrylamide gel electrophoresis (PAGE) and detected by Western blotting using monoclonal (mono) or polyclonal (poly) antibodies against RANKL or against GST. (FIG. 4B) Soluble WT RANKL and RANKL^{G278R} proteins were cross-linked with DSS (+) or PBS (−), run on 12% SDS-PAGE and detected by Western blot using an anti-RANKL polyclonal antibody. (FIG. 4C) HEK 293FT cells were transfected with full-length WT RANKL-FLAG, WT RANKL-Myc and/or RANKL^{G278R}-Myc. Lysates were analyzed in native gels followed by Western blot using an anti-Myc antibody. The protein input was determined in denatured acrylamide gels and Westerns using antibodies against FLAG, Myc and actin. (FIG. 4D) The levels of soluble RANKL were quantified in supernatants of transfected HEK 293FT cells displayed in FIG. 4C. Data shown as mean±SEM of three experiments in duplicate. ***p<0.0001 when compared to WT RANKL-expressing cells. (FIG. 4E) Lysates of transfected HEK 293FT cells were immunoprecipitated with a Myc-specific antibody, and immunoblotted with an anti-FLAG antibody. The protein input was determined in Western blots using antibodies against FLAG, Myc and actin. A representative figure of three independent experiments is shown for Western blots. (FIG. 4F) Different concentrations of RANK-Fc were added to plates coated with either WT GST-RANKL, GST-RANKL^{G278R} or GST and the binding was monitored by fluorescence detection of PE-conjugated goat anti-human IgG. Data shown as mean±SEM of three experiments performed in duplicate.

FIG. 5. Dose-dependent suppression of RANKL-induced osteoclast formation by RANKL^{G278R}. (FIG. 5A) Representative TRAP stain of osteoclast cultures from WT BM cells treated with M-CSF and GST-RANKL in the absence (1:0) or presence of GST-RANKL^{G278R} at various concentrations including 100 ng/ml (1:2), 50 ng/ml (1:1), 25 ng/ml (2:1), or 12.5 ng/ml (4:1). Bar: 100 μm. (FIG. 5B) The number of TRAP+ multinucleated (≧3 nuclei) cells was calculated per well (24-well plate). (FIG. 5C) The nuclei number in TRAP+ multinucleated cells was also calculated. Data shown as mean±SEM of three experiments in duplicate. Each group was compared to that of GST-RANKL (1:0) (**p<0.001, ***p<0.0001).

FIG. 6. G122R substitution abrogates TNF trimer formation, binding to TNFR and bioactivity. (FIG. 6A) Soluble WT TNF and TNF^{G122R} proteins were cross-linked with DSS (+) or PBS (−), run on 12% SDS-PAGE and detected by Western blot using an anti-TNF polyclonal antibody. (FIG. 6B) Different concentrations of p75TNFR-Fc (1-160 ng/ml) were added to plates coated with either soluble TNF, or TNF^{G122R} and the binding was monitored by detection of HRP-conjugated goat anti-human IgG. Data shown as mean±SEM of a representative experiment performed in triplicate. (FIG. 6C) L929 cytotoxicity assay was performed in the presence of WT GST-TNF or GST-TNF^{G122R} at serial dilutions (0.03-4 ng/ml). Data shown as mean±SEM of three experiments performed in triplicate.

FIG. 7. SPD304 inhibits RANKL-induced osteoclastogenesis. (FIG. 7A) Representative TRAP stain of osteoclast cultures from WT BM cells treated with M-CSF and GST-RANKL in the presence of 0.25-2 μM SPD304. Bar: 100 μm. (FIG. 7B) The number of TRAP+ multinucleated (≧3 nuclei) cells was quantitated per well (48-well plate). (FIG. 7C) The nuclei number in TRAP+ multinucleated cells was also calculated. Data shown as mean±SEM of three experiments performed in duplicate. The effect of SPD304 on osteoclast formation was compared to that of untreated cells (*p<0.05, **p<0.001, ***p<0.0001).

FIG. 8. Phenotypic characteristics of osteopetrotic tles/tles mice. (FIG. 8A). Failure of tooth eruption in tles/tles mice. (FIG. 8B). Kaplan-Meier survival curve of control +/+ and tles/+ littermates (n=68), and tles/tles mice (n=20) analyzed in a total of 88 progeny derived from intercrosses between heterozygous tles/+ mice.

FIG. 9. Osteoclast precursor cells from tles/tles mice differentiate into osteoclasts. (FIG. 9A) Numbers of TRAP+ multinucleated osteoclasts per well (24-well plate) derived from BM cultures presented in FIG. 1, Panel C. Data shown as mean±SEM of two experiments (n=4) (P>0.05). (FIG. 9B) TRAP staining of cocultures between splenocytes and primary calvarial osteoblasts (OB) in the presence of 1.25(OH)₂ vitamin D3 and PGE2. Representative data of three experiments performed in triplicate. Bar: 100 μm.

FIG. 10. G278R substitution allows normal RANKL protein production. Total extracts from thymus (T), spleen (S), and bone (B) of WT (Rankl+/+) and Rankl^{tles/tles} mice were prepared and analyzed by Western blotting with specific antibodies against RANKL and actin. The transmembrane form of RANKL (tmRANKL) (45 kD) as well as the soluble form of RANKL (sRANKL) (31 kD) are indicated.

FIG. 11. G122R substitution abrogates TNF multimer formation. Recombinant WT GST-TNF and GST-TNF^{G122R} were resolved on native gel and detected by Western blotting using polyclonal antibodies against RANKL or GST.

FIG. 12. Alignment of several members of the TNF superfamily. Sequences appear in FIG. 12 (SEQ ID NOS: 13-31, respectively).

FIG. 13. Effect of SDP304 on RANKL structure. (Panel A) RANKL dimer with SPD304 located on optimum binding position. G278 is shown with space-filled atoms and SPD304 with doted surface. Cyan and green ribbons represent the two RANKL monomers. Diagram created using PYMOL v1.3. (Panel B) Recombinant WT soluble mouse RANKL (60 ng) was preincubated for 1 hour at 37° C. either with SPD304 at various concentrations from 6-200 μM or without SPD304 (−), resolved on native gel and detected by Western blotting using a polyclonal antibody against RANKL. (Panel C) Recombinant soluble mouse RANKL was preincubated with SPD304 at increasing concentrations from 6-100 μM, was cross-linked with DSS, run on 12% SDS-PAGE and detected by Western blot using an anti-RANKL polyclonal antibody. A representative figure of three independent experiments is shown for Western blots.

FIG. 14. The effect of small molecule inhibitors on RANKL activity. (FIG. 14A) SPD304 at 2 μM inhibits human RANKL activity in osteoclastogenesis assays but induces toxicity in osteoclast precursors (IC50=3.4 μM) as shown in MTT survival assays. (FIG. 14B) SPD304 derivatives and T23 at 5 μM inhibit RANKL activity in osteoclastogenesis assays. (FIG. 14C) The toxic effect of SPD304 derivatives and T23 is examined in MTT survival assays of osteoclast precursors. Data shown are representative of at least three experiments.

FIG. 15. Small molecules disrupt RANKL trimers. PRA224 and T23 were preincubated at various ratios with recombinant soluble human RANKL, were cross-linked and analyzed in 12% PAGE. The RANKL forms were detected using a polyclonal anti-RANKL antibody in Western blots. Data shown are representative of at least three experiments.

FIG. 16. The effect of RANKL peptides on RANKL inhibition. (FIG. 16A) Peptides 1 and 2 at 50 μM inhibit human RANKL activity in osteoclastogenesis assays. (FIG. 16B) Peptide 1 inhibits RANKL trimerization at 50:1 ratio as shown in Western blotting. (FIG. 16C) RANKL peptides inhibit binding of human RANKL to its receptor RANK in a dose dependent manner. Data shown are representative of at least three experiments.

FIG. 17. Inhibition of TNF-induced death in L929 cells. Increasing concentrations of the two compounds (Panel a, compound 1=T23; Panel b, compound 2=PRA224) were used to pre-incubate human TNF before addition to cells for 18 hours. Shown are mean values (n=3) relative to controls (TNF pre-incubated with DMSO). Data shown are representative of at least three experiments. In parallel experiments, the toxicity of the compounds was tested also in L929 cells using the same approach but omitting TNF and actinomycin D from the experimental set-up (Panel c, compound 1; Panel d, compound 2). Shown are mean values (n=3) relative to controls (DMSO-treated cells). Data shown are representative of at least three experiments.

FIG. 18. Disruption of the TNF/TNF-R1 interaction by PRA224. Increasing concentrations of compound 2 (PRA224) were used to pre-incubate human TNF before addition on a TNF-R1 substrate. Binding was measured by ELISA. Shown are mean values (n=2) of one experiment, representative of at least three repeats.

FIG. 19. Reduction of TNF-induced MMP9 release in synovial fibroblasts. Increasing concentrations of the compounds were used to pre-incubate human TNF before used as a stimulus in cultured wild-type synovial fibroblasts for 18 hours (Panel a). Supernatants were collected and MMP activity was visualized by gelatin zymography. The compounds were also used to treat synovial fibroblasts isolated from the human TNF-transgenic mouse, which release MMP9 without stimulation (Panel b). In both Panels a and b, DMSO was used as a control.

FIG. 20. TNF cross-linking experiment. Human TNF was incubated with different molar ratios of the compounds, or DMSO as a control, cross-linked with BS3, and subjected to SDS-PAGE. This was followed by Western blotting to detect the various TNF multimers.

FIG. 21. G249R substitution abrogates BAFF trimer formation and binding to BAFF receptor. (FIG. 21A) Various amount (1.2, 0.6, 0.3 μg) of soluble WT BAFF and BAFF^{G249R} proteins were cross-linked with DSS (+) or PBS (−), run on 12% SDS-PAGE and detected by Western blot using an anti-BAFF polyclonal antibody. (FIG. 21B) Different concentrations of BAFF receptor (3-400 ng/ml) were added to plates coated with either soluble BAFF, or BAFF^{G249R}. The RANKL binding to RANK was monitored by detection of HRP-conjugated goat anti-human IgG. Data shown as mean±SEM of a representative experiment.

FIG. 22. Structure of SPD304 analogues.

FIG. 23. Structure of T23 and derivatives. Compound 1 corresponds to T23.

### DETAILED DESCRIPTION

The disclosure relates to the identification of a functional amino acid critical for ligand trimerization and bioactivity within the TNF ligand superfamily. A conserved glycine residue was found to be involved in RANKL trimer assembly. Further demonstrated is that RANKL trimerization can be inhibited by mutating an amino acid in the RANKL trimerization domain or by providing a compound that binds to the trimerization domain.

Describes is a chemically induced recessive mutation in the Rankl gene that causes severe osteopetrosis in mice similar to Rankl deficient mice. This loss-of-function mutation induces a glycine to arginine substitution (G278R) at the inner hydrophobic F beta-strand of the RANKL monomer that not only inhibits trimer assembly but also exerts a dominant negative effect on the wild-type (WT) RANKL assembly and function.

Although it has been previously proposed that RANKL trimerization involves intersubunit interactions among 43 residues, scattered mainly within the ten highly conserved beta-strands of each monomer,^{[6]} it is shown here for first time that a single amino acid substitution is sufficient to completely disrupt trimer assembly. Previous attempts at identifying functional RANKL residues have been based on predictions made on the crystal structure of RANKL/RANK. Such studies have been exclusively concentrated in amino acids interacting with the RANK receptor such as the Glu225, Arg222, and Asp299 residues,^{[36]} where their substitution leads to a dramatic decrease on binding to RANK and subsequent inability to promote osteoclast formation. The forward genetics approach described in the disclosure is the first to identify and characterize a critical amino acid substitution that results in protein inactivation and subsequently to osteopetrosis in vivo.

RANKL is a member of the TNF (Tumor Necrosis Factor) superfamily. TNF superfamily proteins are important regulators of innate and adaptive immune responses and developmental events and proteins constitute an important class of cytokines that participate in a variety of cellular and intracellular signaling processes. The cognate receptors of the TNF superfamily ligands make up a related superfamily of receptors.

The TNF superfamily proteins are synthesized as type 2 membrane proteins and fold into conserved beta-pleated sheet structures. The three-dimensional structures of TNF superfamily members are very similar, made up of a sandwich of two anti-parallel beta-sheets each formed by five anti-parallel beta strands with the “jelly roll” or Greek key topology. The inner sheet is formed from beta strands A, A′, H, C, and F, while the outer sheet is formed from beta strands B, B′, D, E, and G.

In addition, all characterized members of the family assemble into noncovalently associated trimers. The biologically active trimers exist in both membrane-bound and soluble cleaved forms. Most TNF superfamily members form homotrimers, although lymphotoxin-beta, for example, can form heterotrimers with lymphotoxin-alpha. Similarly, APRIL and BAFF also form both homotrimers and heterotrimers together (Daridon et al., Autoimmunity Reviews, Volume 7, Issue 4, February 2008, pages 267-271).

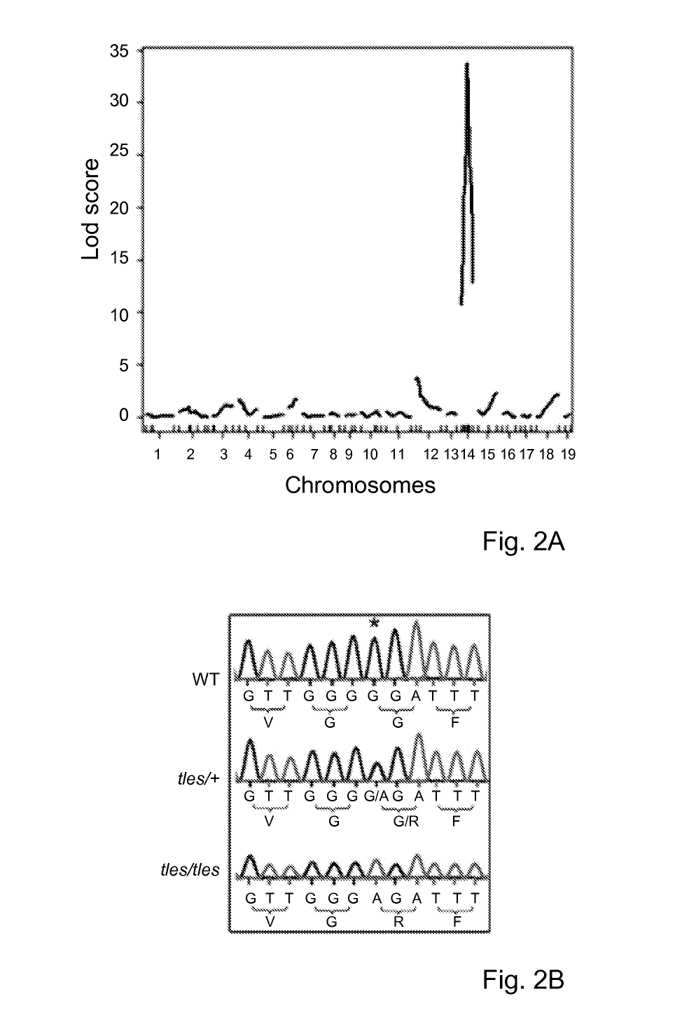

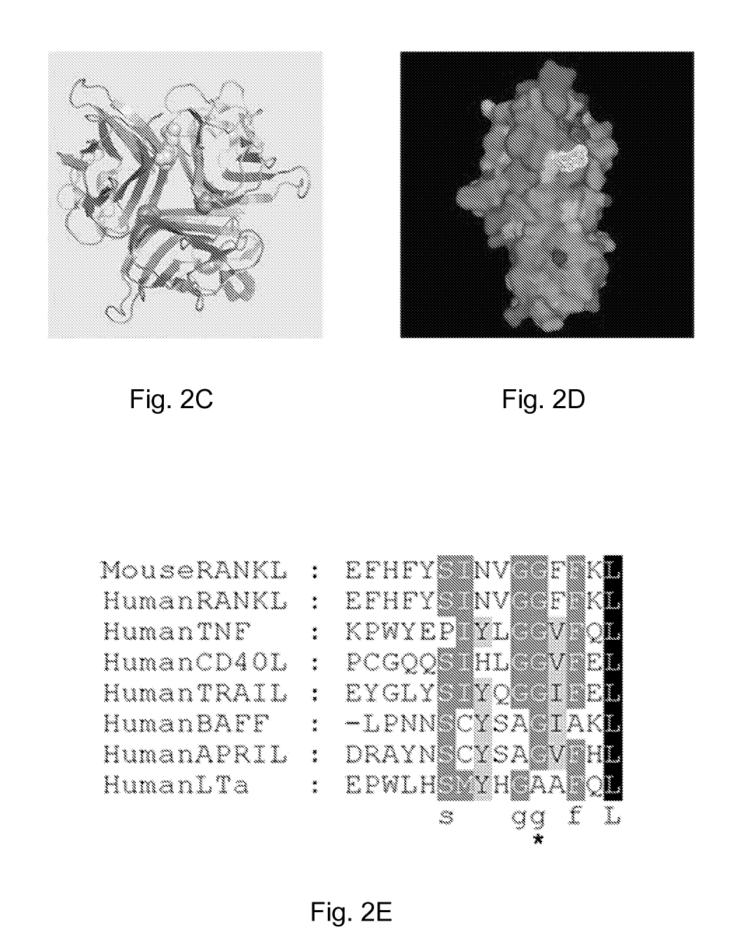

The RANKL^{G278R} mutation identified herein is located at the hydrophobic F beta-strand, which is 100% conserved between human and mouse RANKL. The F beta-strand is part of the inner A′AHCF β-sheet that is involved in intersubunit association. The introduction of a positive charge as well as a long side chain is expected to disrupt the hydrophobic interface and create steric hindrances causing packing inefficiencies (FIG. 2D). Biochemical analysis on recombinant soluble RANKL has revealed that functional trimers or multimers are not detected for the RANKL^{G278R} protein, confirming our structure-based prediction regarding the trimerization inability of RANKL^{G278R}. Instead, the studies described herein reveal the presence of monomers as well as the formation of RANKL^{G278R} aggregates. Since formation of a functional RANKL trimer is prerequisite for receptor binding, RANKL^{G278R} is unable to bind and activate RANK that is required for the stimulation of the downstream signaling cascades leading to osteoclast differentiation, activation and survival.

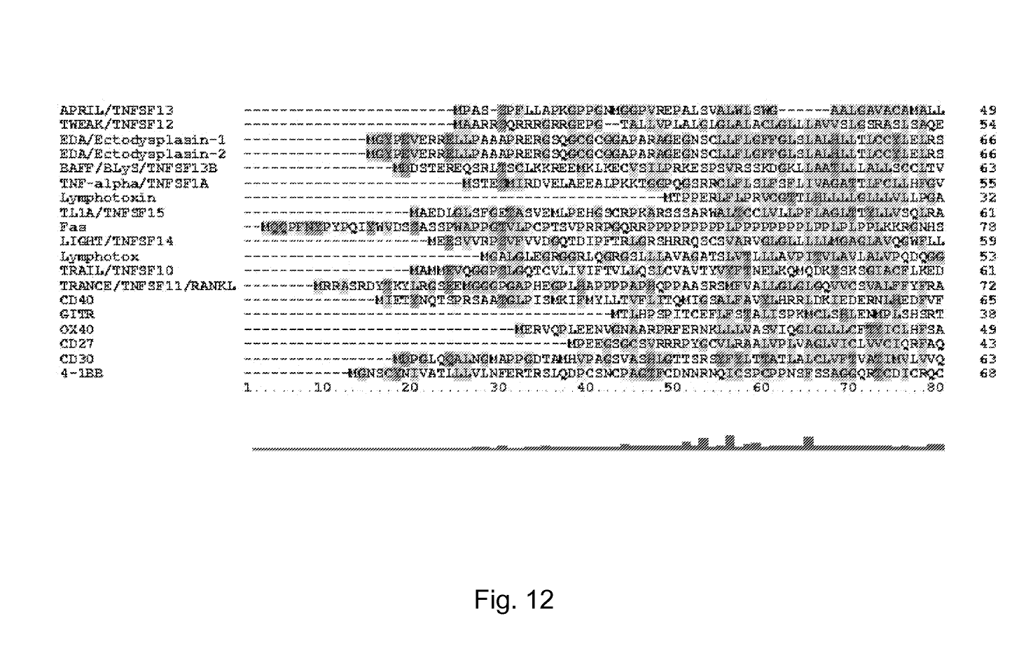

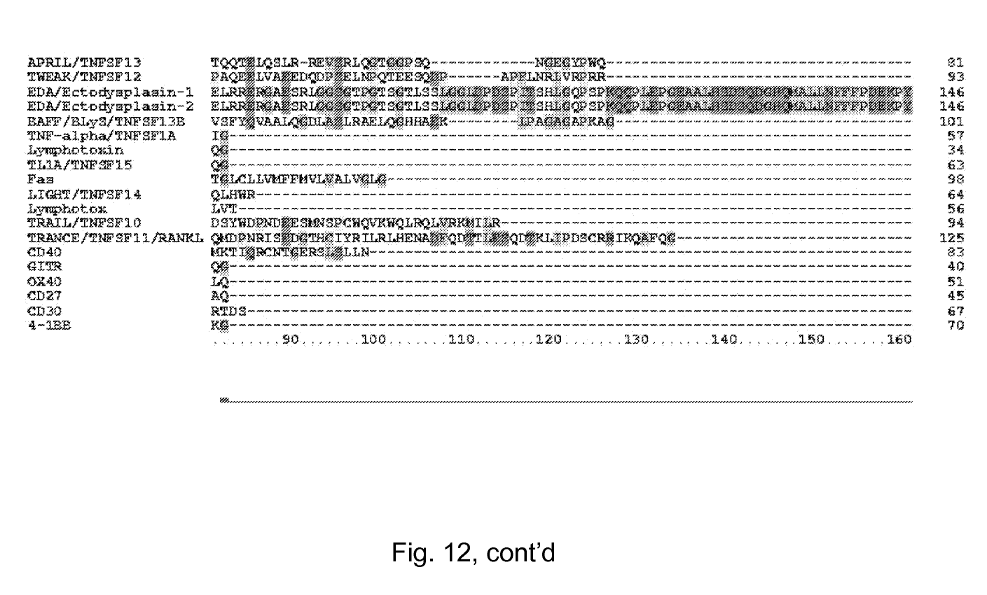

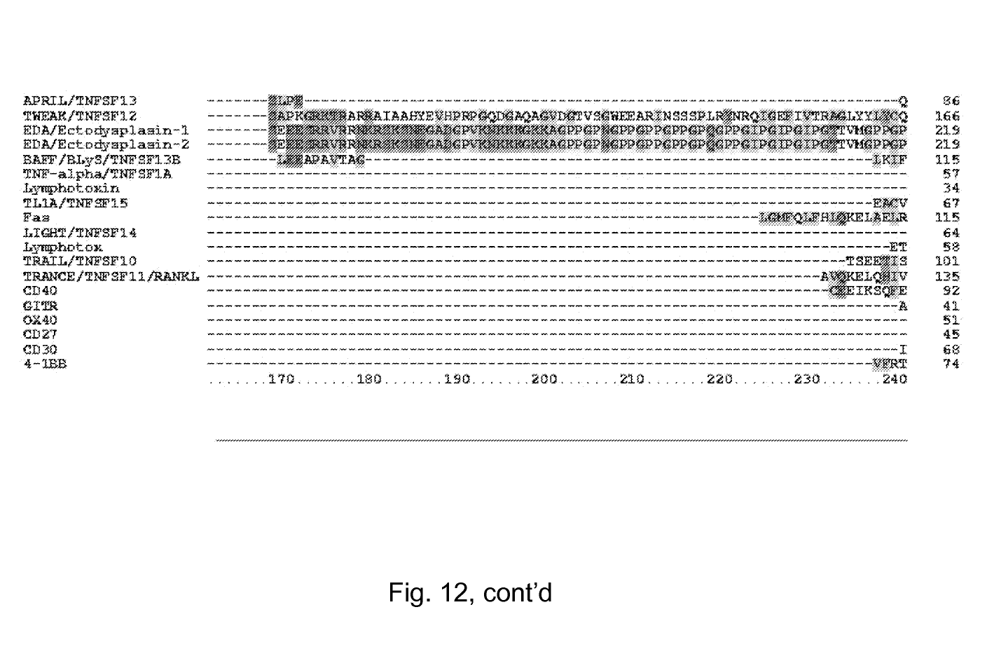

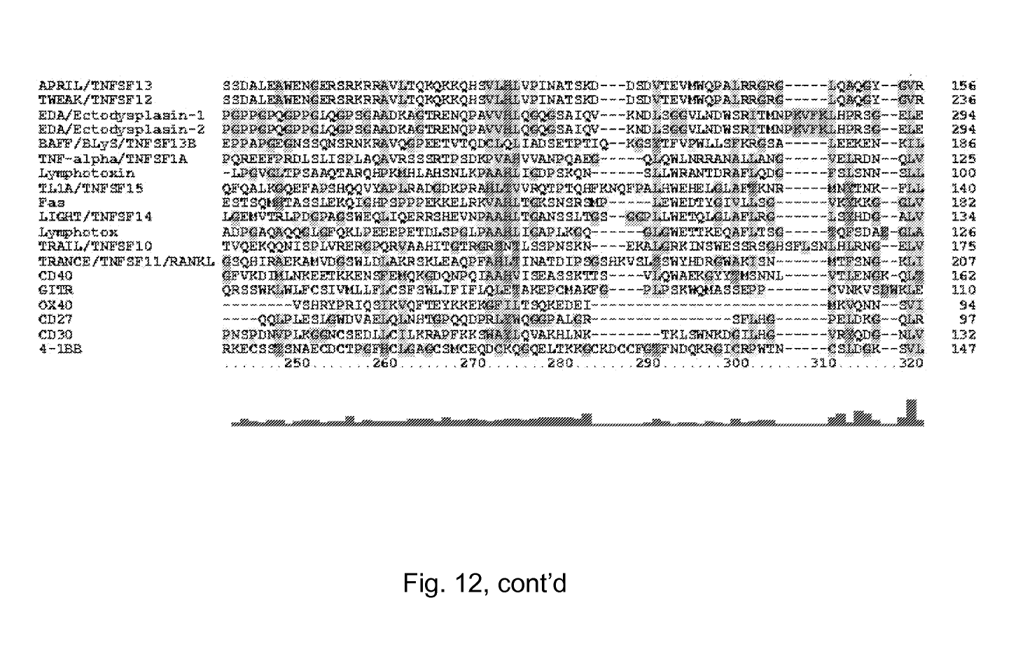

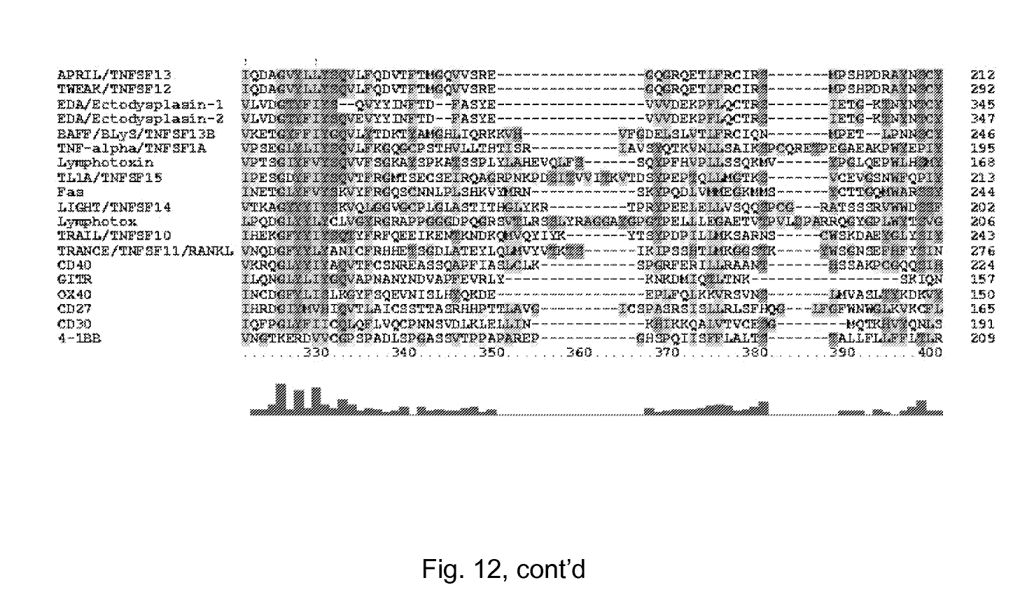

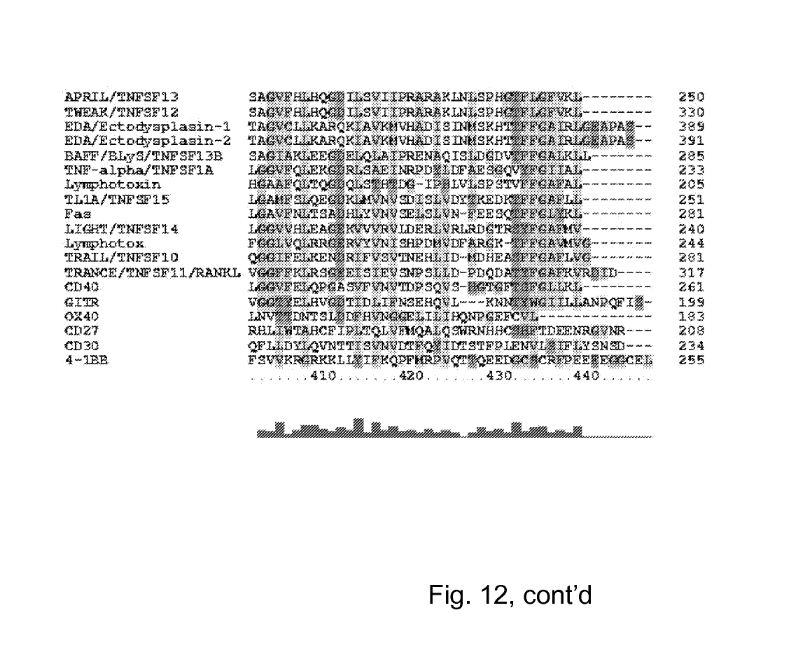

The sequence identity between members of the TNF superfamily is around 20-30% and members share a number of conserved residues as depicted in FIG. 12. Interestingly, the glycine residue identified in RANKL for its involvement is trimerization is conserved among the TNF superfamily. This residue is also conserved among several members of the C1q family, which also trimerize, such as C1qA, C1Qb, C1Qc, Precerebellin, and CollVIIIa2 (see, FIG. 2 of Bodmer et al., 2002, Trends in Biochemical Sciences, which is hereby incorporated by reference). The disclosure further demonstrates that a similar residue substitution in TNF, G122R, abrogates TNF trimer formation, binding to the p75TNF receptor and bioactivity, highlighting its importance within the TNF superfamily.

Accordingly, also provided is a method for inhibiting trimerization of a TNF superfamily member polypeptide comprising contacting the polypeptide with a compound that inhibits trimerization of the polypeptide, herein referred to as a “trimerization inhibitor.” The polypeptide may be any polypeptide belonging to the TNF superfamily that forms a trimer, for example, TNF-alpha, lymphotoxin-alpha, lymphotoxin-beta, Fas ligand (FasL), TRAIL, CD40 ligand (CD40L), CD30 ligand, CD27 ligand, Ox40 ligand, APRIL, BAFF (BLyS), 4-IBBL, BAFF, and RANKL. Preferably, the polypeptide is TNF-alpha or RANKL.

The polypeptide may also belong to a related family, such as the C1q family. Many of these proteins—which also form trimers or multimers of trimers—have been implicated in development, and immunological and physiological homeostasis. Preferred members of the C1q family are C1qA, C1Qb, C1Qc, Precerebellin, and CollVIIIa2.

Preferably, the method comprises contacting a cell expressing a TNF superfamily member polypeptide with a trimerization inhibitor. Preferably, the cell is a mammalian cell, more preferably a human cell. In some embodiments, the method is carried out in vitro. The trimerization inhibitors as described herein may therefore be used as tools to study the TNF superfamily signaling pathways.

Preferably, the trimerization inhibitor binds a TNF superfamily, or related family, member polypeptide at the F beta-strand. The disclosure provides a number of trimerization inhibitors including compounds and derivatives of formula I, TNF superfamily polypeptide or fragments thereof, and T23. “F beta-strand” binders are useful in the methods described herein.

Preferably, the trimerization inhibitor as described herein is selected from a) a compound that binds to the TNF superfamily, or related family, member polypeptide at the F beta-strand, preferably at the glycine residue that corresponds to position 279 in human RANKL and b) a TNF superfamily, or related family, member polypeptide or fragment thereof, preferably having a dominant negative mutation in the trimerization domain (herein referred to as the “dominant negative polypeptide”). Preferably, the trimerization inhibitor also induces the disassociation of already formed trimers.

Preferably, the trimerization inhibitor is 6,7-Dimethyl-3-[[methyl[2-[methyl[[1-[3-(trifluoromethyl)phenyl]-1H-indol-3-yl]methyl]amino]ethyl]amino]methyl]-(4H-1-Benzopyran-4-one) (also known as SPD304) or functional derivatives thereof. As used herein, a functional derivative can bind a TNF superfamily polypeptide and act as a trimerization inhibitor. Preferably, the derivatives are selected from PRA123, PRA224, PRA333, PRA738, and PRA828, more preferably PRA828, most preferably PRA224.

The formation or disassociation of trimers can be measured by any number of assays known to one of skill in the art, including mass spectrometry (see, e.g., reference 35 herein), intrinsic fluorescence measurements, dynamic light scattering, and the assays described in the Examples (Example 4). The effect on trimerization can also be observed by measuring the binding of a TNF superfamily ligand to its cognate receptor, as receptor binding is dependent of ligand trimerization, or by measuring receptor activity (see, e.g., Examples 4 and 5). In some embodiments, the compound is provided to a cell. Preferably, the provision of the compound to a cell inhibits trimerization of the TNF superfamily, or related family, member polypeptide by at least 10, at least 20, at least 30, at least 40, at least 50, at least 60, at least 70, at least 80, or at least 90%.

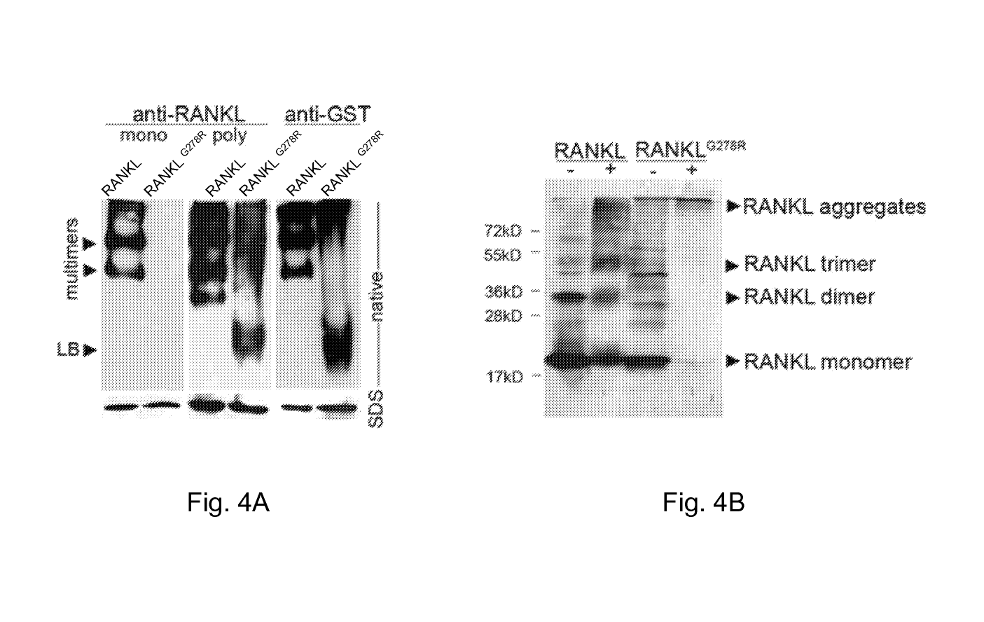

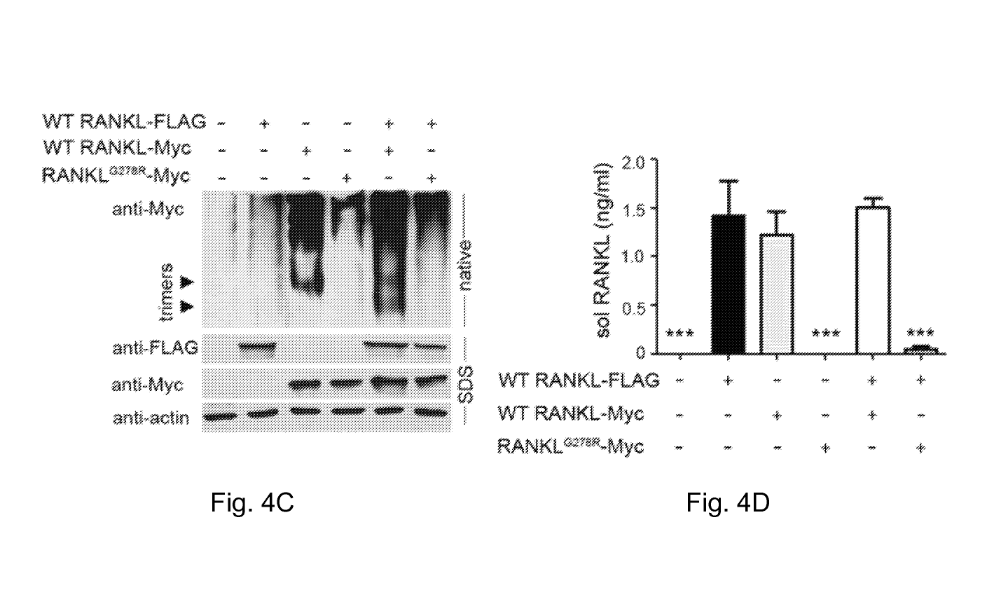

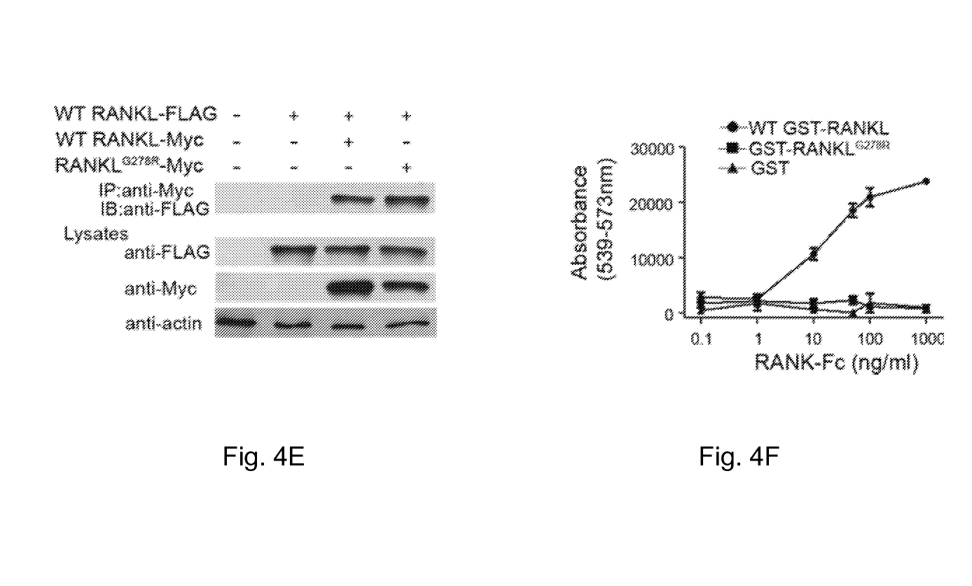

The inhibition of trimerization also encompasses the induction of non-functional RANKL aggregates and/or the increase of monomers. Accordingly, the detection of an increase in aggregates indicates an inhibition of trimerization. This increase in aggregates can also be detected as a decrease in soluble RANKL (see, FIG. 4D). In a preferred embodiment, the inhibition of trimerization results in the decrease of trimeric soluble RANKL protein by at least 30, at least 40, at least 50, at least 60, at least 70, at least 80, or at least 90%.

Further provided are methods for inhibiting osteoclast formation or decreasing bone loss in an individual comprising administering an effective amount of a compound that inhibits trimerization of RANKL. Preferably, the trimerization inhibitor is used to treat inflammation-induced and/or immune-mediated loss of bone and/or cartilage and/or RANKL-mediated osteoporosis.

The trimerization inhibitor may be administered prophylactically, i.e., before bone loss occurs, in order to prevent bone loss or it may be administered after bone loss has occurred in order to decrease further bone loss. Preferably, the trimerization inhibitor is administered to an individual such that bone loss is decreased by at least 5, 10, 20, 30, 40, 50, or 60% compared to non-treatment.

Provided are methods for preventing, treating, or reducing symptoms in an individual afflicted with osteoporosis, preferably postmenopausal osteoporosis, rheumatoid arthritis, multiple myeloma, bone metastasis, juvenile osteoporosis, osteogenesis imperfecta, hypercalcemia, hyperparathyroidism, osteomalacia, osteohalisteresis, osteolytic bone disease, osteonecrosis, Paget's disease of bone, bone loss due to rheumatoid arthritis, inflammatory arthritis, osteomyelitis, periodontal bone loss, bone loss due to cancer, age-related loss of bone mass, osteopenia, and inflammatory bowel syndrome comprising administering an effective amount of a compound that inhibits trimerization of RANKL.

Preferably, the individual (or subject) is a mammal, more preferably a human.

Rheumatoid arthritis (RA) is a chronic systemic inflammatory disorder with an unknown cause characterized by invasive synovial hyperplasia leading to progressive joint destruction. Bone erosion begins in the early stages of the disease and results in severe deformity of the affected joints, which impairs the normal activity and quality of life of patients. Rheumatoid arthritis can be associated with elevated RANKL in T-cells, synovial fibroblasts, and bone marrow stroma;

The BXD2 mouse strain develops arthritis with bone erosions, synovial hyperplasia with mononuclear cell infiltration, and joint deformation. These mice also have high levels of rheumatoid factor and anti-DNA auto-antibodies. In this model, inhibition of RANKL completely prevented bone loss and partially protected against cartilage loss (Y. Wu et al., 2005, Arthritis Rheum. 52:3257-3268).

Periodontal diseases are chronic infectious inflammatory diseases characterized by increased leukocyte infiltration into the periodontal lesions. This infiltration results in the secretion of a number of cytokines, which leads to the destruction of periodontal tissues including alveolar bone (M. A. Taubman et al., 2001, Crit. Rev. Oral Biol. Med. 12:125-135).

RANKL expressed by either osteoblasts or infiltrating T cells in response to bacterial infection is involved in alveolar bone destruction in periodontal diseases. RANKL messenger RNA is up-regulated in gums from patients with severe periodontitis.

Periprosthetic bone loss leading to aseptic loosening of implants is one of the most challenging complications of joint replacement surgeries. Osteoclast-like multinucleated cells are observed in the bone-implant interface of the loosened joints and the fibroblastic cells in the perioprosthetic tissues have been shown to induce the differentiation of normal human peripheral blood mononuclear cells into mature osteoclasts by a mechanism that involves both RANKL and TNF-alpha (A. Sabokbar et al., 2005, J. Orthop. Res. 23:511-519).

Hypercalcemia is a late stage complication of cancer, disrupting the body's ability to maintain normal levels of calcium, resulting in calcium deposit in the kidneys, heart conditions and neural dysfunction and occurs most frequently in patients with cancers of the lung and breast. Hypercalcemia also occurs in patients with multiple myeloma, cancers of the head and neck, sarcoma, cancers of unknown primary origin, lymphoma, leukemia, melanoma, renal cancer, and gastrointestinal cancers (e.g., esophageal, stomach, intestinal, colon and rectal cancers). RANK and RANKL play a role in bone loss associated with cancers. When RANKL+ myeloma cells are injected into C57BL mice, the mice develop bone disease characterized by a marked decrease in cancellous bone volume in the tibial and femoral metaphyses, increased osteoclast formation, and radiologic evidence of osteolytic bone lesions.

Specific blockade of RANKL prevents the skeletal complications in various animal models of myeloma and suppressed bone resorption in patients with myeloma bone disease. Treatment of myelomatous SCID-human mice with a RANK-Fc fusion protein reduced myeloma-induced bone resorption and resulted in a greater than 80% reduction in paraprotein. Treatment resulted in a reduced number of osteoclasts, but had no effect on the apoptosis and proliferation of myeloma cells, suggesting that the anti-myeloma effect of RANKL inhibitors is associated with inhibition of osteoclast activity (Yaccodby et al., 2002, Br. J. Haematol., 116:278-290).

Other cancer indications, which the compounds described herein can treat include, but are not limited to: hematologic neoplasias and neoplastic-like conditions, for example, Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia.

Some primary tumors and metastatic malignant tumors, such as breast cancer and lung cancer, invade bone tissues. Osteoclasts are primarily responsible for the osteolysis observed in these patients and there's evidence that in patients with severe osteolysis, the RANKL/OPG ratio is increased (Y. Wittrant et al., Biochim. Biophys. Acta 2004, 1704:49-57; E. Greimaud et al., 2003, Am. J. Pathol. 163:2021-2031).

The RANKL/RANK/OPG system has also been reported to be involved in bone destruction in breast cancer cells, prostate cancer cells, and other metastatic bone tumors (S. Kitazawa et al., 2002, J. Pathol. 198:228-236; H. R. Park et al., 2003, J. Korean. Med. Sci. 18:541-546; J. Zhang et al., 2001, J. Clin. Invest. 107:1235-1244; E. T. Keller et al., 2001, Cancer Metastasis Rev., 20:333-349).

Some patients with Juvenile Paget's Disease have mutations in the OPG gene, which result in undetectable serum levels of OPG and large increases in soluble RANKL levels. This disorder is a rare disease with an autosomal inheritance pattern, and it displays various deformities of long bones and vertebral column, which increase in severity during adolescence. (M. P. Whyte et al., 2002, N. Engl. J. Med. 347:175-184; T. Cundy et al., 2002, Hum. Mol. Genet. 11:2119-2127; B. Chong et al., 2003, J. Bone Miner. Res. 18:2095-2104).

Compounds that bind to TNF superfamily, or related family, member polypeptides at the glycine residue that corresponds to position 279 in human RANKL and their method of preparation are described, for example, in WO2008/142623, which is hereby incorporated by reference.

The compounds include a compound of formula 1, or a stereoisomer thereof, tautomer thereof, or mixture thereof in any ratio; a pharmaceutically acceptable salt, pharmaceutically acceptable solvate, or pharmaceutically acceptable polymorph thereof;

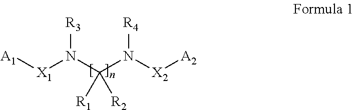

wherein:
A₁ and A₂ are independently a substituted or unsubstituted heterocyclic system selected from:

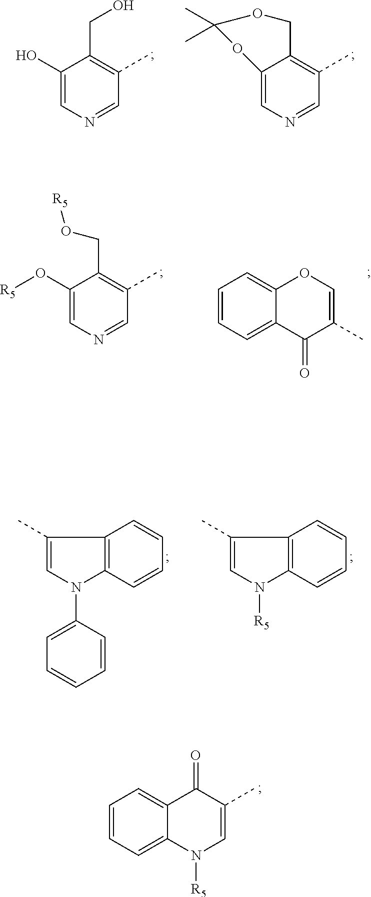

wherein the dotted line indicates the point of attachment, R₅ is hydrogen or (C₁-C₄)-alkyl group and the rings of the heterocyclic systems herein above may be substituted with groups selected from (C₁-C₄)-alkyl, (C₁-C₄)-alkoxy, hydroxyl, hydroxy-(C₁-C₄)-alkyl (e.g., hydroxymethyl or 1-hydroxyethyl or 2-hydroxyethyl), and fluoroalkyl (e.g., CF₃);
X₁ and X₂ are independently a carbonyl group or a methylene (—CH₂—) group; n is an integer from 2-4;
R₁ and R₂ are independently, hydrogen or (C₁-C₄)-alkyl group;
R₃ and R₄ are independently, hydrogen or (C₁-C₄)-alkyl group; and
R₃ and R₄ can optionally form a ring system; with a proviso that when A₁ and A₂ are 1-(3-(thfluoromethyl)phenyl)-1H-indole and 6,7-dimethyl-4H-chromen-4-one respectively and X₁ and X2 are independently a methylene (—CH₂—) group, R₃ and R₄ form a ring system.

Preferably, A₁ and A₂ are independently a substituted or unsubstituted phenyl group wherein the substituents on the phenyl ring are selected from:
(C1-C4)-alkyl, fluoroalkyl such as CF₃, hydroxyl, (C₁-C₄)-alkoxy, benzyloxy and hydroxy-(C₁-C₄)-alkyl;
X₁ and X₂ are independently a carbonyl group or a methylene (—CH₂—) group;
n is an integer from 2-4;
R₁ and R₂ are independently, hydrogen or (C₁-C₄)-alkyl group;
R₃ and R₄ are independently, hydrogen or (C₁-C₄)-alkyl group; and
R₃ and R₄ can optionally form a ring system.

Preferably, the compound is selected from:
3,3′-(ethane-1,2-diylbis(methylazanediyl))bis(methylene)bis(6,7-dimethyl-4H-chromen-4-one)dihydrochloride;
5,5′-(ethane-1,2-diylbis(methylazanediyl))bis(methylene)bis(4-(hydroxymethyl)-2-methylpyridin-3-ol)dihydrochloride;
6,7-Dimethyl-3-((methyl(2-(methyl((2,2,8-trimethyl-4H-[1,3] dioxino[4,5-c]pyhdin-5-yl)methyl)amino)ethyl)amino)methyl)-4H-chromen-4-one dihydrochloride;
1,4-Bis((1-(3-(trifluoromethyl) phenyl)-1H-indol-3-yl)methyl)piperazine dihydrochloride;
6,7-Dimethyl-3-((4-((1-(3-(trifluoromethyl)phenyl)-1H-indol-3-yl)methyl)piperazin-1-yl)methyl)-4H-chromen-4-one dihydrochloride;
N₁,N₂-bis(4-(benzyloxy)-3-methoxybenzyl)ethane-1,2-diamine dihydrochloride;
N,N′-(ethane-1,2-diyl)bis(2-hydroxybenzamide)dihydrochloride;
N,N′-(propane-1,3-diyl)bis(2-hydroxybenzamide)dihydrochloride; and
4-Hydroxy-N-(2-(2-hydroxybenzamido)ethyl)-3-methoxybenzamide dihydrochloride.

Most preferably, the compound is 6,7-Dimethyl-3-[[methyl[2-[methyl[[1-[3-(trifluoromethyl)phenyl]-1H-indol-3-yl]methyl]amino]ethyl]amino]methyl]-(4H-1-Benzopyran-4-one) (also known as SPD304) or functional derivatives thereof.

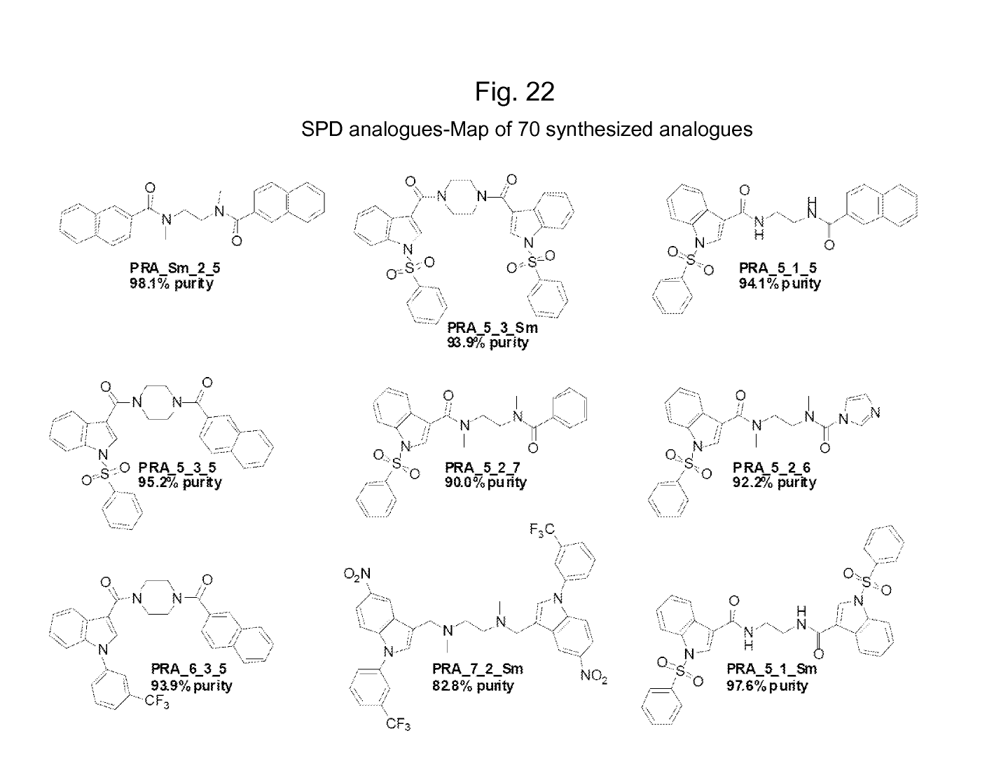

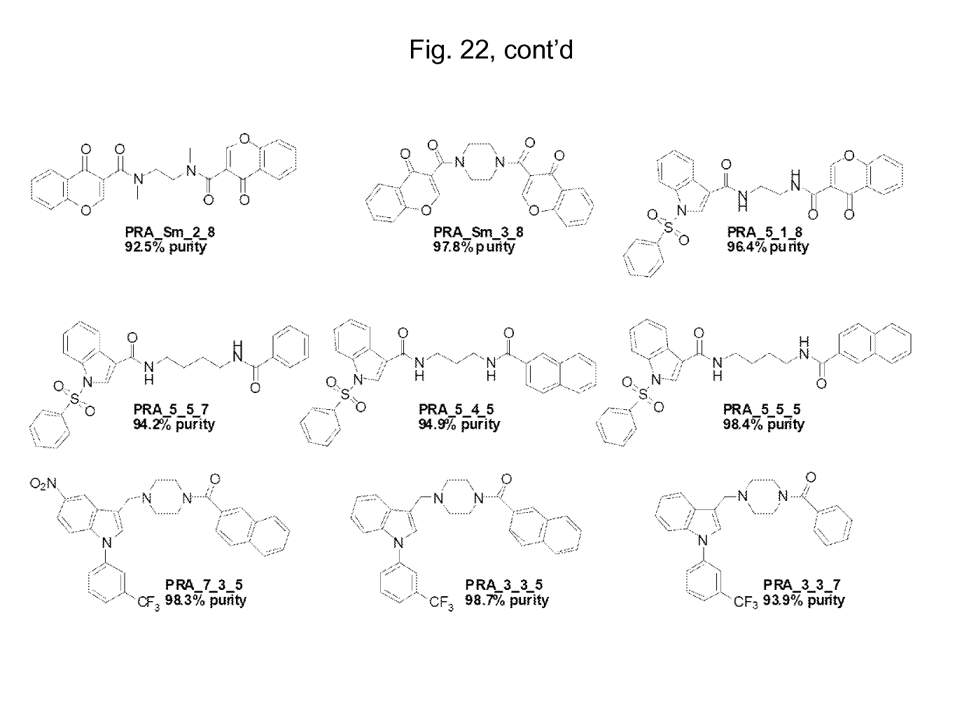

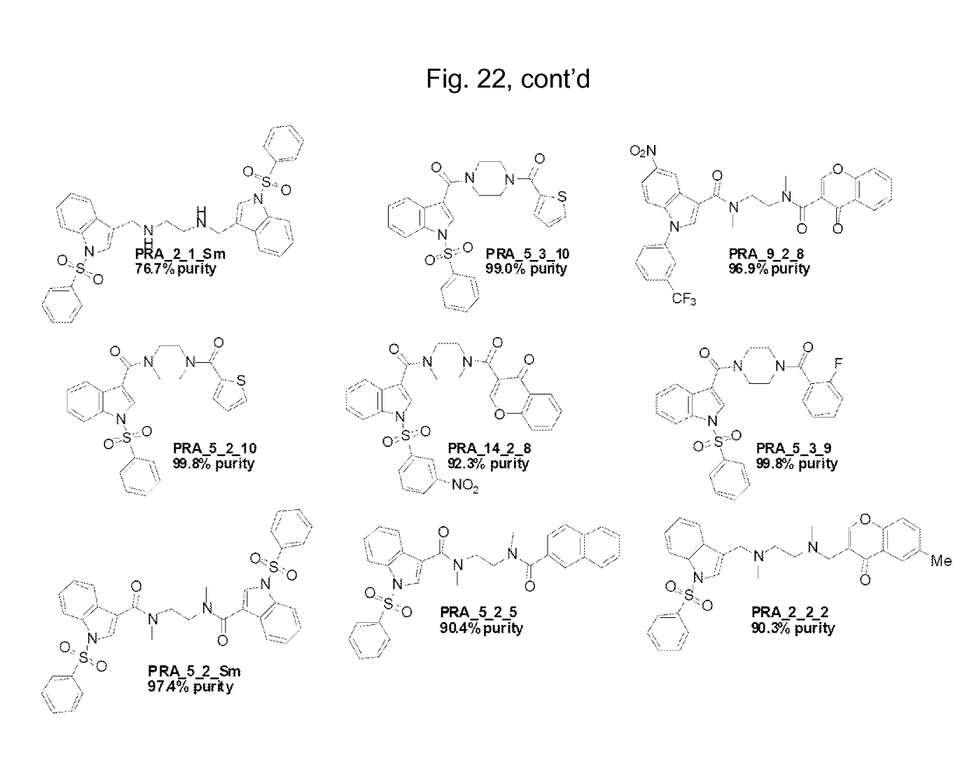

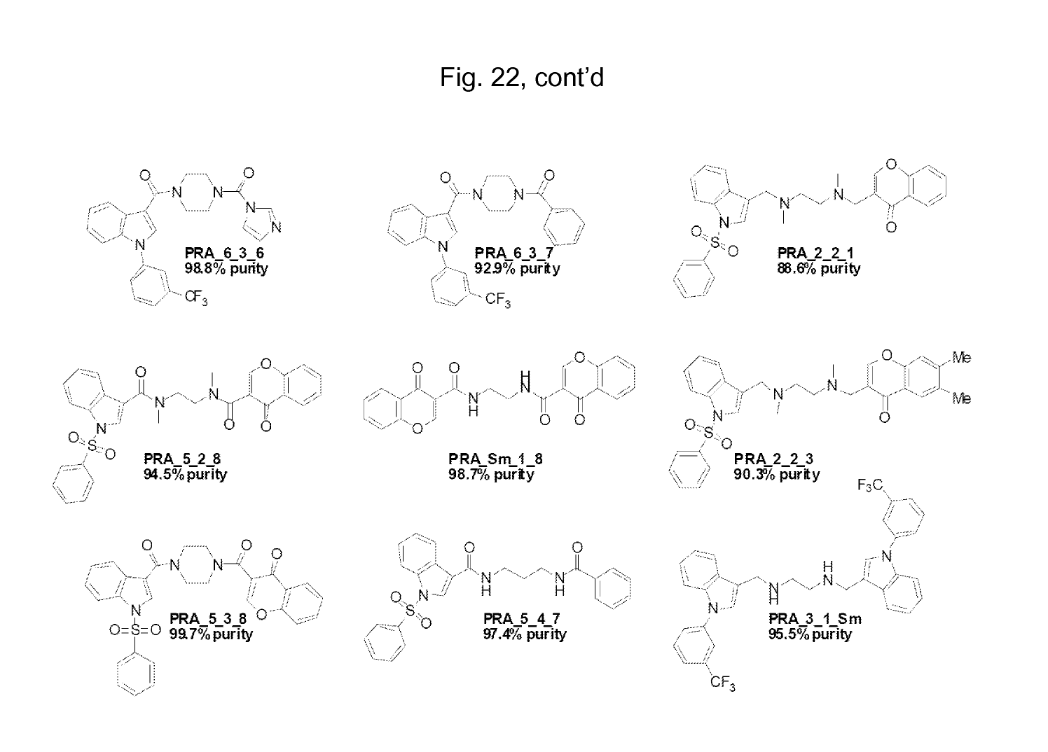

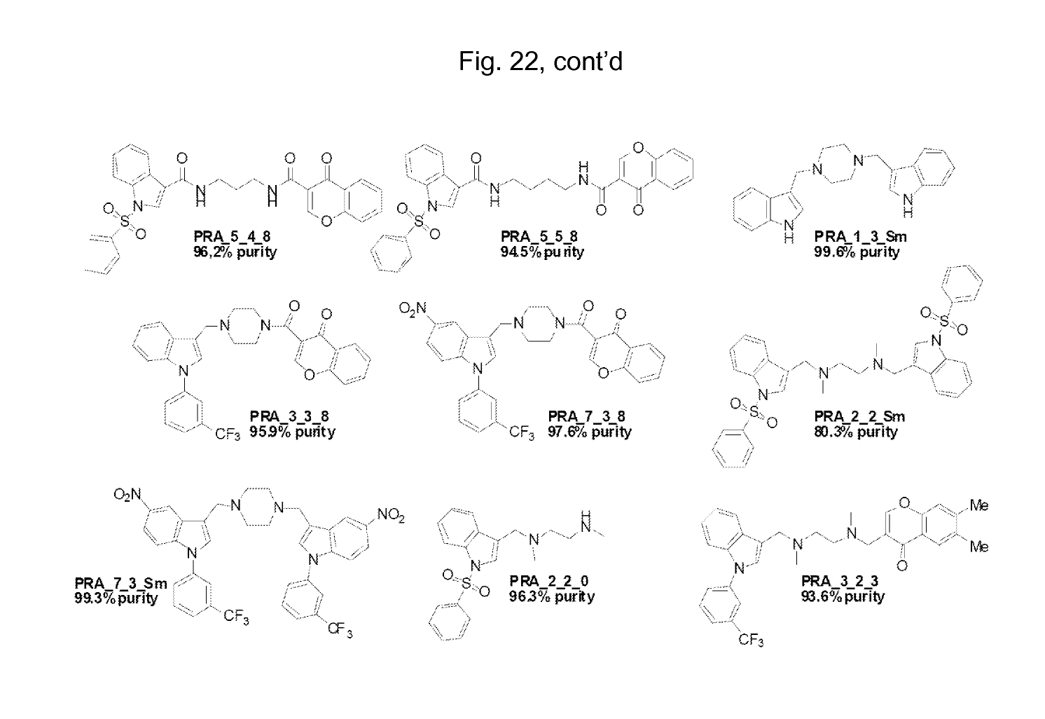

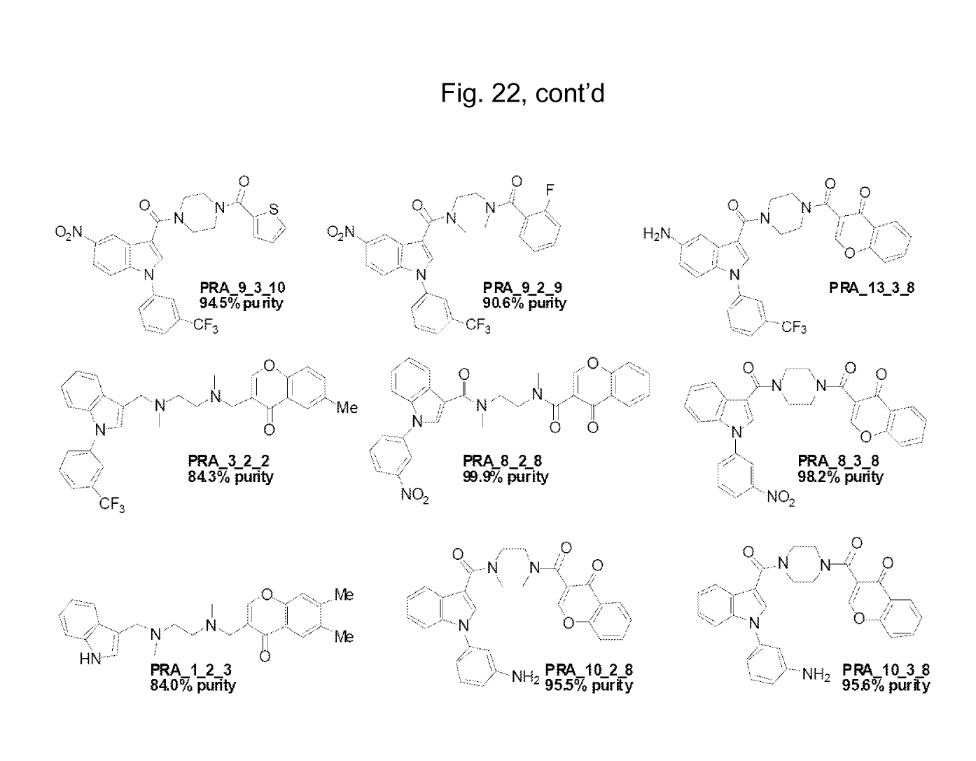

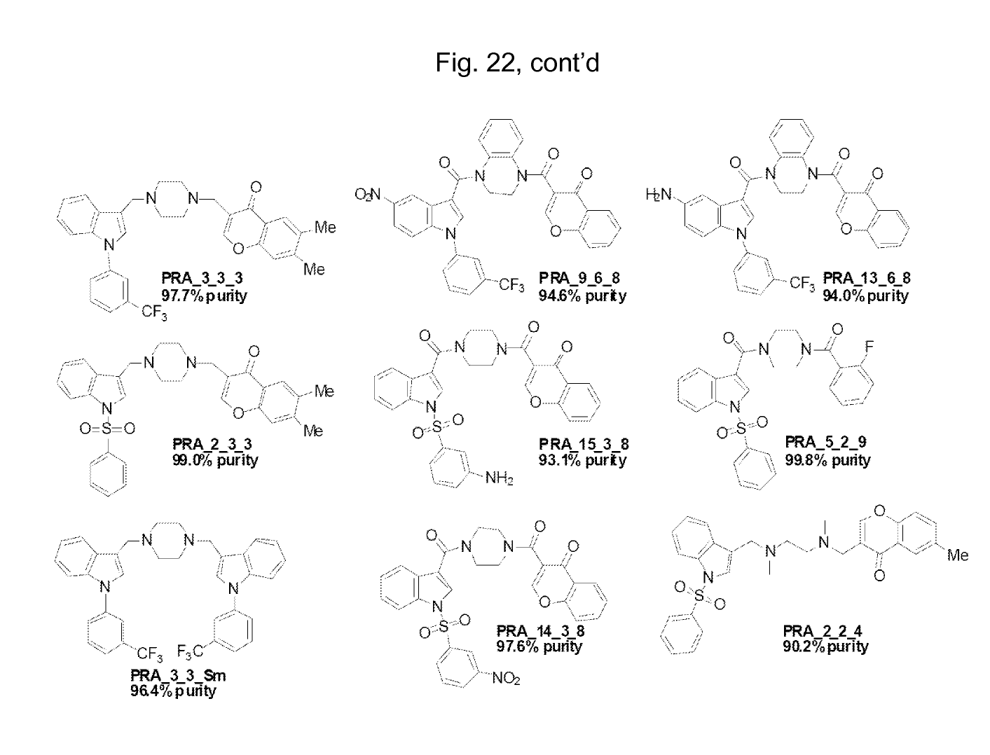

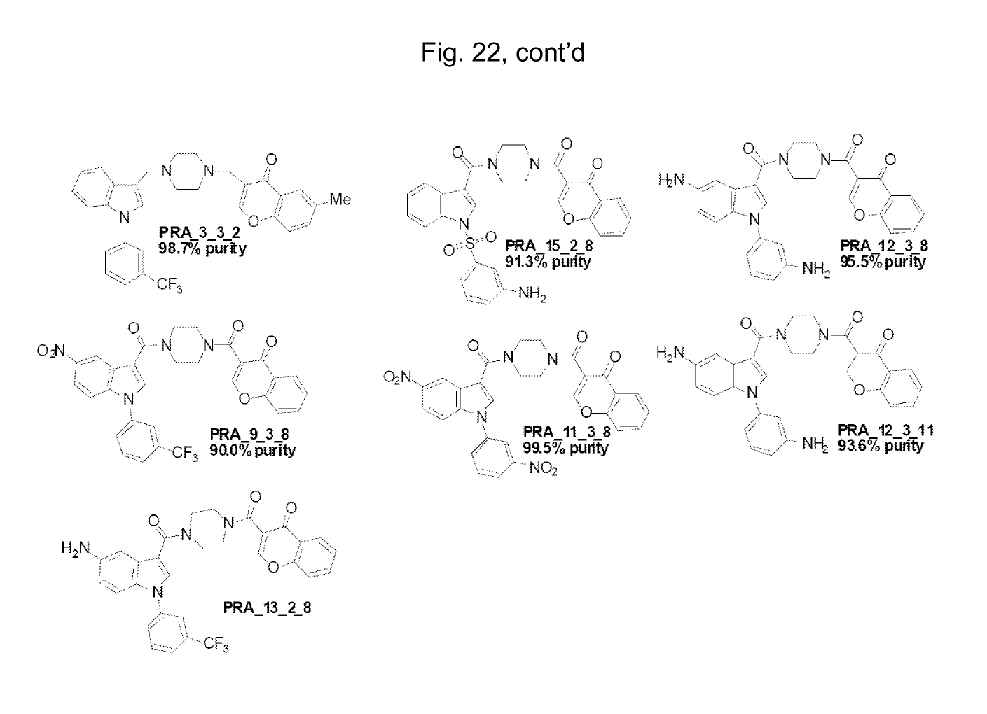

In order to identify compounds with improved properties, particularly lower toxicity, new SPD304 derivatives were synthesized and tested for their ability to inhibit TNF and RANKL in vitro. FIG. 22 depicts active compounds.

The disclosure thus also includes new compounds not disclosed in WO2008/142623. Such compounds include those having formula 1:

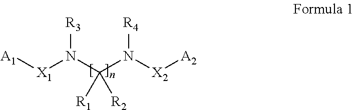

wherein:
A₁ and A₂ are independently a substituted or unsubstituted heterocyclic system selected from:

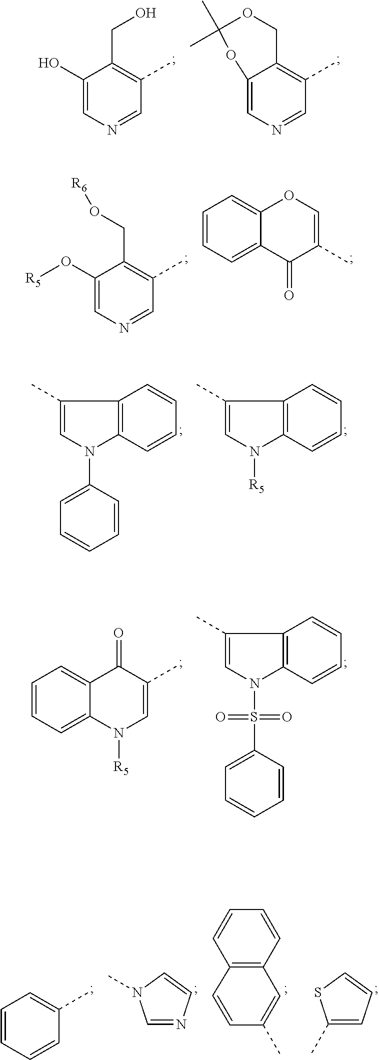

wherein the dotted line indicates the point of attachment, R₅ is hydrogen or (C₁-C₄)-alkyl group and the rings of the heterocyclic systems herein above are unsubstituted or substituted with one or more groups selected from (C₁-C₄)-alkyl, (C₁-C₄)-alkoxy, hydroxyl, hydroxy-(C₁-C₄)-alkyl (e.g., hydroxymethyl or 1-hydroxyethyl or 2-hydroxyethyl), fluoroalkyl (e.g., CF₃), halide (e.g., fluoro); nitro (NO₂) and amino (NH₂);
X₁ and X₂ are independently a carbonyl group or a methylene (—CH₂—) group; n is an integer from 2-4;
R₁ and R₂ are independently, hydrogen or (C₁-C₄)-alkyl group;
R₃ and R₄ are independently, hydrogen or (C₁-C₄)-alkyl group; or
wherein R₃ and R₄ are a single (C₁-C₈)-hydrocarbon group connecting the two nitrogen atoms of formula 1, which group may be selected from saturated hydrocarbon (e.g., C₂H₄) and aromatic hydrocarbon (e.g., phenyl);
with the proviso that when A₁ and A₂ are independently a substituted or unsubstituted heterocyclic system selected from;

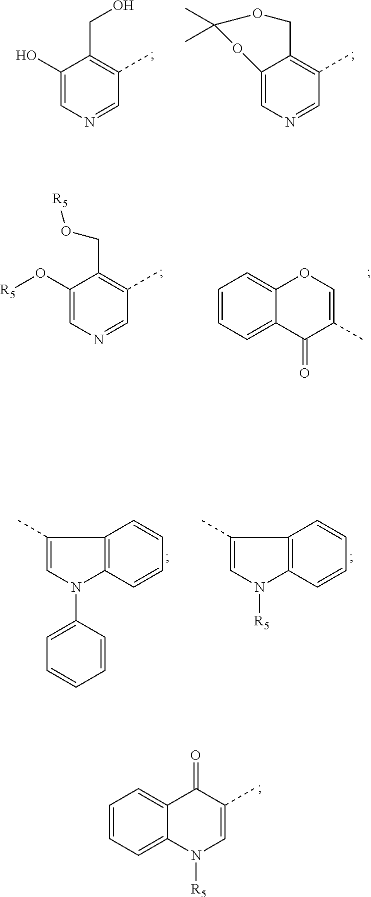

the heterocyclic systems are substituted with one or more groups selected from halide (e.g., fluoro); nitro (NO₂) and amino (NH₂).

In the methods disclosed herein, a compound of formula 1, or a stereoisomer thereof, tautomer thereof, or mixture thereof in any ratio; a pharmaceutically acceptable salt, pharmaceutically acceptable solvate, or pharmaceutically acceptable polymorph thereof may be used, including:

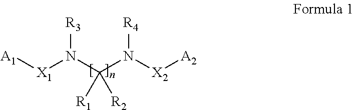

wherein:
A₁ and A₂ are independently a substituted or unsubstituted heterocyclic system selected from:

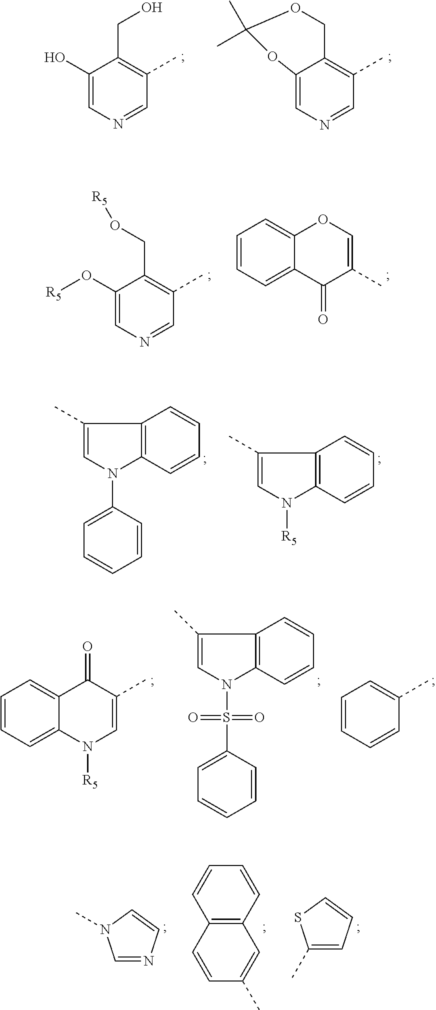

wherein the dotted line indicates the point of attachment, R₅ is hydrogen or (C₁-C₄)-alkyl group and the rings of the heterocyclic systems herein above are unsubstituted or substituted with one or more groups selected from (C₁-C₄)-alkyl, (C₁-C₄)-alkoxy, hydroxyl, hydroxy-(C₁-C₄)-alkyl (e.g., hydroxymethyl or 1-hydroxyethyl or 2-hydroxyethyl), fluoroalkyl (e.g., CF₃), halide (e.g., fluoro); nitro (NO₂) and amino (NH₂);
X₁ and X₂ are independently a carbonyl group or a methylene (—CH₂—) group; n is an integer from 2-4;
R₁ and R₂ are independently, hydrogen or (C₁-C₄)-alkyl group;
R₃ and R₄ are independently, hydrogen or (C₁-C₄)-alkyl group;
or wherein R₃ and R₄ are a single (C₁-C₈)-hydrocarbon group connecting the two nitrogen atoms of formula 1, which group may be selected from saturated hydrocarbon (e.g., C₂H₄) and aromatic hydrocarbon (e.g., phenyl).

It has been previously shown that the small molecule SDP304, which interacts with TNF at the glycine residue at position 122, effectively inhibits TNF trimerization and function.^{[35]} Accordingly, when SDP304 is used as the trimerization inhibitor, the TNF superfamily member is not TNF-alpha.

Preferably, when the trimerization inhibitor is a compound of formula 1 as described above, or a stereoisomer thereof, tautomer thereof, or mixture thereof in any ratio; a pharmaceutically acceptable salt, pharmaceutically acceptable solvate, or pharmaceutically acceptable polymorph thereof; the TNF superfamily member polypeptide is not TNF-alpha. More preferably, when the trimerization inhibitor is a compound that binds to the TNF superfamily member polypeptide in the F beta-strand, the TNF superfamily member polypeptide is not TNF-alpha.

Demonstrated is that 6,7-Dimethyl-3-[[methyl[2-[methyl[[1-[3-(trifluoromethyl)phenyl]-1H-indol-3-yl]methyl]amino]ethyl]amino]methyl]-(4H-1-Benzopyran-4-one) effectively inhibits RANKL-induced ex vivo osteoclast formation, suggesting a surprising possible common mechanism of action to that of TNF inhibition.

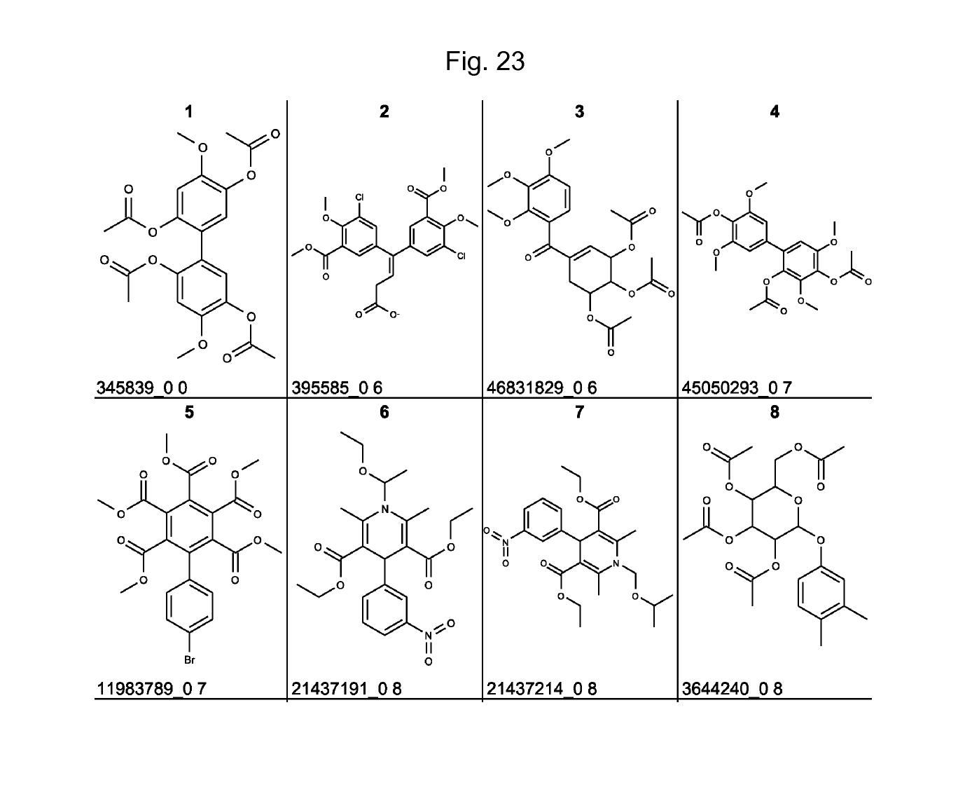

In one aspect, T23 and its functional derivatives are provided as TNF superfamily inhibitors. T23 was identified based on in silico screening method to identify molecules that bind the F-strand of the TNF superfamily. As demonstrated in the examples, T23 (compound 1 of FIG. 23) inhibits trimerization of both RANKL and TNF. Functional derivatives of T23 are further provided (compound 2-1000 of FIG. 23). As used herein, functional derivatives of T23 bind a TNF superfamily polypeptide, preferably TNF or RANKL, preferably the F-strand of the polypeptide, and inhibit its trimerization. The functional derivatives of T23 were identified by searching a chemical database for neighbors of T23 in the chemical space. These derivatives are predicted to have similar binding and, therefore, similar functional properties as T23.

It will be appreciated by those skilled in the art that the compounds described herein may also be provided in the form of their pharmaceutically acceptable salts or solvates thereof. The pharmaceutically acceptable salts of the compounds are, in particular, salts that are non-toxic, or that can be used physiologically. The disclosure furthermore includes all solvates of the compounds, for example, hydrates, and the solvates formed with other solvents of crystallization, such as alcohols, ethers, ethyl acetate, dioxane, DMF, or a lower alkyl ketone, such as acetone, or mixtures thereof.

In one aspect, a dominant negative TNF superfamily, or related family, member polypeptide or fragment thereof is provided (i.e., “dominant negative polypeptide”) is provided. Preferably, the dominant negative polypeptide comprises a mutation in the trimerization domain. Preferably, the dominant negative polypeptide is a wild-type TNF superfamily peptide.

As used herein, a dominant negative polypeptide refers to a polypeptide that affects the function of the normal, wild-type form of the polypeptide. In preferred embodiments, the dominant negative polypeptides adversely affect the ability of wild-type TNF family polypeptides to form trimers. It has been previously shown that trimer assembly within the TNF ligand family constitutes a dynamic process, where subunits can be exchanged.^{[40]} Although not wishing to be bound by theory, this phenomenon could explain the dominant negative effect exerted by the RANKL^{G278R} variant.

Dominant negative polypeptides are useful as trimerization inhibitors of the TNF superfamily or related families such as members of the C1q family that forms trimers. Preferably, the dominant negative polypeptide is selected from TNF-alpha, lymphotoxin-alpha, lymphotoxin-beta, Fas ligand (FasL), TRAIL, CD40 ligand (CD40L), CD30 ligand, CD27 ligand, Ox40 ligand, APRIL, BAFF (BLyS), 4-IBBL, BAFF, TWEAK, ectodysplasin-1, ectodysplasin-2, LIGHT, and RANKL, more preferably, the polypeptide is TNF-alpha or RANKL.

Preferably, the dominant negative polypeptide is non-naturally occurring.

Preferably, the dominant negative polypeptide is provided as an isolated and/or purified polypeptide. As used herein, “isolated” means that the polypeptides are separated from other components of either (a) a natural source, such as a plant or cell, preferably bacterial culture, or (b) a synthetic organic chemical reaction mixture. Preferably, via conventional techniques, the compounds of the disclosure are purified. As used herein, “purified” means that when isolated, the isolate contains at least about 80%, preferably at least about 90%, more preferably at least about 95% and even more preferably at least about 98%, of the polypeptide by weight of the isolate.

Preferably, the dominant negative polypeptide is the same family member as the TNF superfamily member whose trimerization is to be inhibited. It is contemplated that dominant negative polypeptides of one species, e.g., RANKL from mouse, can be used to inhibit the trimerization of a TNF superfamily polypeptide in another species, e.g., RANKL from human. A skilled person will appreciate that cross-species inhibition is possible based on the conservation of sequence between species. Preferably, the dominant negative polypeptide is from the same species as the TNF superfamily member to be inhibited.

Preferably, the dominant negative polypeptide comprises at least one amino acid mutation in its trimerization domain that inhibits the ability of the polypeptide to form trimers. The mutation may be an amino acid deletion, insertion, or substitution, preferably the mutation is a substitution. Preferred amino acid residues in the trimerization domain include the tyrosine residue that corresponds to position 307 in human RANKL (Y227 in human TNF-alpha and Y151 in soluble human TNF-alpha), the asparagine, valine, glycine, and glycine residues that correspond to positions 276-279 in human RANKL (195-198 in human TNF-alpha and 119-122 in soluble human TNF-alpha), as well as the leucine residue that corresponds to position 57 in soluble human TNF-alpha, the tyrosine residue that corresponds to position 59 in soluble human TNF-alpha, the serine residue that corresponds to position 60 in soluble human TNF-alpha, and the glutamine residue that corresponds to position 61 in soluble human TNF-alpha. It is clear to a skilled person that mutations can be made in other TNF superfamily, and related family, members at positions that correspond to those described in RANKL and TNF-alpha.

Preferably, the dominant negative polypeptide comprises a mutation in the glycine residue that corresponds to position 279 in human RANKL. This position corresponds to 215 in APRIL, 295 in TWEAK, 348 in Ectodysplasin-1, 350 in Ectodysplasin-2, 249 in BAFF, 246 in TRAIL, 227 in CD40L, 198 in TNF-alpha, 122 in soluble human TNF, 205 in LIGHT, and 209 in Lymphotoxin. Preferably, the mutation is an amino acid substitution, more preferably a non-conservative amino acid substitution.

Preferably, the dominant negative polypeptide comprises non-conservative modifications (e.g., substitutions). By “non-conservative” modification herein is meant a modification in which the wild-type residue and the mutant residue differ significantly in one or more physical properties, including hydrophobicity, charge, size, and shape. For example, modifications from a polar residue to a nonpolar residue or vice-versa, modifications from positively charged residues to negatively charged residues or vice versa, and modifications from large residues to small residues or vice versa are non-conservative modifications. For example, substitutions may be made that more significantly affect: the structure of the polypeptide backbone in the area of the alteration; the charge or hydrophobicity of the molecule at the target site; or the bulk of the side chain. The substitutions that, in general, are expected to produce the greatest changes in the polypeptide's properties are those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine. In a preferred embodiment, the dominant negative polypeptide comprises a mutation in the glycine residue that corresponds to position 279 in human RANKL, wherein glycine is substituted for arginine, lysine, histidine, ornithine, methyllysine, or acetyllysine. Preferably, the glycine is substituted for arginine.

It is also contemplated that the dominant negative polypeptide as disclosed herein may include one or more amino acid analogs such as D-amino acid, di-amino acid, and/or beta-amino acid.

The dominant negative polypeptides may also contain additional amino acid modifications that those related to disrupting trimerization. Examples include amino acid substitutions introduced to enable soluble expression in E. coli, amino acid substitutions introduced to optimize protein stability, and amino acid substitutions introduced to modulate immunogenicity. The polypeptides may also comprise epitope or purification tags or be fused to other therapeutic proteins or proteins such as Fc or serum albumin for pharmacokinetic purposes.

As used herein, dominant negative polypeptides include non-full length polypeptides such as the soluble form of the polypeptides, i.e., lacking the transmembrane domain. An exemplary soluble polypeptide is the RANKL soluble polypeptide: KLEAQPFAHLTINATDIPSGSHKVSLSSWYHDRGWAKISNMTFSNGKLIVNQDGFYYLY ANICFRHHETSGDLATEYLQLMVYVTKTSIKIPSSHTLMKGGSTKYWSGNSEFHFYSINV GGFFKLRSGEEISIEVSNPSLLDPDQDATYFGAFKVRDID (SEQ ID NO:1).

Preferably, the dominant negative polypeptide or a fragment thereof is a peptide comprising HFYSINVGGFFK (SEQ ID NO:4) or HFYSINVGRFFK (SEQ ID NO:5). Preferably, the dominant negative polypeptide or a fragment thereof is a peptide comprising an amino acid sequence at least 90% identical to HFYSINVGGFFK (SEQ ID NO:4) or HFYSINVGRFFK (SEQ ID NO:5). Preferably, the peptide has between 12-100, more preferably between 12-50, most preferred between 12-30 amino acids.

As is apparent to one of skill in the art, dominant negative polypeptides useful in the methods disclosed herein also include functional fragments of the polypeptides. As used herein, “functional fragments” refers to fragments that inhibit trimerization. At a minimum, such functional fragments comprise the F beta strand residues (corresponding to amino acid residues 270-282 of human RANKL). Preferably, the functional fragments comprise an amino acid sequence at least 90% identical to amino acid residues 270-282 of human RANKL. Additional residues may also be present in order to provide stability or influence the pharmokinetics of the fragments. In some embodiments, the fragment is a retro-inverso analogue or a circular peptide.

In some aspects, the disclosure provides a polypeptide or a functional fragment thereof comprising an amino acid sequence having at least 80, at least 90, at least 95, or at least 99% identity to the human RANKL sequence: MRRASRDYTKYLRGSEEMGGGPGAPHEGPLHAPPPPAPHQPPAASRSMFVALLGLGLG QVVCSVALFFYFRAQMDPNRISEDGTHClYRILRLHENADFQDTTLESQDTKLIPDSCRRI KQAFQGAVQKELQHIVGSQHIRAEKAMVDGSWLDLAKRSKLEAQPFAHLTINATDIPSG SHKVSLSSWYHDRGWAKISNMTFSNGKLIVNQDGFYYLYANICFRHHETSGDLATEYL QLMVYVTKTSIKIPSSHTLMKGGSTKYWSGNSEFHFYSINVGXFFKLRSGEEISIEVSNPS LLDPDQDATYFGAFKVRDID (SEQ ID NO:2), wherein X is not glycine.

Preferably, a polypeptide or a functional fragment thereof is provided comprises an amino acid sequence having at least 80, at least 90, at least 95, or at least 99% identity to the soluble form of the human RANKL sequence: KLEAQPFAHLTINATDIPSGSHKVSLS SWYHDRGWAKISNMTFSNGKLIVNQDGFYYLY ANICFRHHETSGDLATEYLQLMVYVTKTSIKIPSSHTLMKGGSTKYWSGNSEFHFYSINV GXFFKLRSGEEISIEVSNPSLLDPDQDATYFGAFKVRDID (SEQ ID NO:3), wherein X is not glycine.

The polypeptide or functional fragment thereof preferably reduces RANKL trimer assembly by at least 10, at least 20, at least 30, at least 40, at least 50, at least 60, at least 70, at least 80, or at least 90%.

In some embodiments, the fragments of TNF superfamily member polypeptides that induce a dominant negative effect are fragments of a wild-type sequence of a TNF superfamily member.

The dominant negative polypeptides may derive from any source, although mammalian polypeptides are preferred. Suitable mammals include, rodents (rats, mice, hamsters, guinea pigs, etc.), primates, farm animals (including sheep, goats, pigs, cows, horses, etc.); and in the most preferred embodiment, from humans.

The mutations resulting in the dominant negative polypeptides may be generated by any number of techniques known to one of skill in the art. These include, e.g., alanine scanning (see, U.S. Pat. No. 5,506,107), gene shuffling (WO 01/25277), and site-directed PCR mutagenesis.

In addition to providing the dominant negative polypeptides as described herein, the disclosure also provides isolated nucleic acids encoding the polypeptides, vectors containing such nucleic acids, and host cells and expression systems for transcribing and translating such nucleic acids into polypeptides.

Accordingly, provided are nucleic acids encoding the dominant negative polypeptides as disclosed herein. The nucleic acids may be operably linked to additional sequences such as promoter sequences, ribosomal binding sites, transcriptional start and stop sequences, translational start and stop sequences, and enhancer or activator sequences. Promoter sequences encode either constitutive or inducible promoters. The promoters may be either naturally occurring promoters or hybrid promoters. Hybrid promoters, which combine elements of more than one promoter, are also known in the art, and are useful in the disclosure. In a preferred embodiment, the promoters are strong promoters, allowing high expression in cells, particularly mammalian cells, such as the CMV promoter, particularly in combination with a Tet regulatory element.

Vectors comprising the nucleic acids are also provided. A “vector” is a recombinant nucleic acid construct, such as plasmid, phase genome, virus genome, cosmid, or artificial chromosome, to which another DNA segment may be attached. The term “vector” includes both viral and nonviral means for introducing the nucleic acid into a cell in vitro, ex vivo or in vivo. Non-viral vectors include plasmids, liposomes, electrically charged lipids (cytofectins), DNA-protein complexes, and biopolymers. Viral vectors include retrovirus, adeno-associated virus, pox, baculovirus, vaccinia, herpes simplex, Epstein-Barr and adenovirus vectors. Vector sequences may also contain one or more regulatory regions, and/or selectable markers useful in selecting, measuring, and monitoring nucleic acid transfer results (transfer to which tissues, duration of expression, etc.).

Cells comprising the nucleic acids or vectors comprising nucleic acids are also provided. The method of introduction is largely dictated by the targeted cell type include, e.g., CaPO₄ precipitation, liposome fusion, lipofectin, electroporation, dextran-mediated transfection, calcium phosphate precipitation, polybrene mediated transfection, protoplast fusion, viral infection, encapsulation of the polynucleotide(s) in liposomes, and direct microinjection of the DNA into nuclei. The nucleic acids may stably integrate into the genome of the host cell (for example, with retroviral introduction, outlined below), or may exist either transiently or stably in the cytoplasm (i.e., through the use of traditional plasmids, utilizing standard regulatory sequences, selection markers, etc.).

Dominant negative polypeptides as described herein may be produced by culturing a host cell transformed with an expression vector containing nucleic acid encoding a dominant negative polypeptide. Appropriate host cells include yeast, bacteria, archaebacteria, fungi, and insect and animal cells, including mammalian cells. Of particular interest are Drosophila melangaster cells, Saccharomyces cerevisiae and other yeasts, E. coli, Bacillus subtilis, SF9 cells, C129 cells, 293 cells, Neurospora, BHK, CHO, COS, Pichia pastoris, etc.

Preferably, the polypeptides are expressed in mammalian cells. Mammalian expression systems are also known in the art, and include retroviral systems.

Suitable cell types include tumor cells, Jurkat T cells, NIH3T3 cells, CHO, and Cos, cells.

Preferably, the polypeptides are expressed in bacterial systems. Bacterial expression systems are well known in the art.

In a preferred embodiment, the nucleic acid encoding the dominant negative polypeptide may also be used in gene therapy. In gene therapy applications, genes are introduced into cells in order to achieve in vivo synthesis of a therapeutically effective genetic product, for example, for replacement of a defective gene. “Gene therapy” includes both conventional gene therapy, where a lasting effect is achieved by a single treatment, and the administration of gene therapeutic agents, which involves the one time or repeated administration of a therapeutically effective DNA or mRNA.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11:205-210 (1993)).

Further provided are non-human animals, preferably mammals, comprising nucleic acids encoding dominant negative polypeptides. Methods for introducing nucleic acids into animals are known to one of skill in the art and include standard transgenic techniques such as introducing the nucleic acid into an undifferentiated cell type, e.g., an embryonic stem (ES) cell. The ES cell is injected into a mammalian embryo, where it integrates into the developing embryo. Insertion of the nucleic acid construct into the ES cells can be accomplished using a variety of methods well known in the art including, for example, electroporation, microinjection, and calcium phosphate treatment. The embryo is implanted into a foster mother for the duration of gestation.

Transgenic animals comprise a heterologous nucleic acid sequence present as an extrachromosomal element or stably integrated in all or a portion of its cells, especially in germ cells. During the initial construction of the animal, “chimeras” or “chimeric animals” are generated, in which only a subset of cells have the altered genome. Chimeras are primarily used for breeding purposes in order to generate the desired transgenic animal. Animals having a heterozygous alteration are generated by breeding of chimeras. Male and female heterozygotes are typically bred to generate homozygous animals.

Also provided is the generation of a novel autosomal recessive osteopetrosis model in mice (tles), characterized by defective tooth eruption due to a complete lack in osteoclasts. These mice carry a loss-of-function allele of Rankl that corresponds to a single amino acid substitution from glycine to arginine (G278R) at the extracellular inner hydrophobic F β-strand of RANKL. Unlike previously described mice having Rankl null alleles,^{[13, 14]} the various forms of the RANKL protein are present in the homozygous Rankl^{tles/tles} mutant mice. Since, no differences were detected in the skeletal phenotype between tles and Rankl null alleles, our results indicate that a single amino acid change is sufficient to cause osteopetrosis without interfering with RANKL expression.

The ties osteopetrotic model closely resembles RANKL-mediated human ARO as in both cases the RANKL protein is produced but is inactive due to mutations at the extracellular bioactive region. Three RANKL mutations have been identified in ARO, M199K, del145-177AA, and V277WfX5;^{[27]} the single amino acid substitution M199K is located within a highly conserved domain, the deletion 145-177 removes a region essential for osteoclastogenesis whereas the frameshift deletion V277WfX5 is predicted to lack the trimerization domain. Notably, the Rankl^{tles/tles} mice constitute a unique animal model useful in the validation of new therapeutic approaches in ARO.

Further provided are pharmaceutical preparations comprising a trimerization inhibitor as disclosed herein and a pharmaceutically acceptable carrier, filler, preservative, adjuvant, solubilizer, diluent and/or excipient is also provided. Such pharmaceutically acceptable carrier, filler, preservative, adjuvant, solubilizer, diluent and/or excipient may for instance be found in Remington: The Science and Practice of Pharmacy, 20th Edition, Baltimore, Md., Lippincott Williams & Wilkins, 2000.

When administering the pharmaceutical preparations hereof to an individual, it is preferred that the compound is dissolved in a solution that is compatible with the delivery method. For intravenous, subcutaneous, intramuscular, intrathecal and/or intraventricular administration it is preferred that the solution is a physiological salt solution. Preferred are excipients capable of forming complexes, vesicles and/or liposomes that deliver such a compound as defined herein in a vesicle or liposome through a cell membrane. Many of these excipients are known in the art. Suitable excipients comprise polyethylenimine (PEI) or similar cationic polymers, including polypropyleneimine or polyethylenimine copolymers (PECs) and derivatives, ExGen 500, synthetic amphiphils (SAINT-18), LIPOFECTIN™, DOTAP and/or viral capsid proteins that are capable of self-assembly into particles that can deliver such compounds, to a cell.

Active ingredients of the disclosure can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the disclosure. The disclosure thus encompasses single unit dosage forms suitable for oral administration such as, but not limited to, tablets, capsules, gel caps, and caplets that are adapted for controlled-release.

Actual dosage levels of the pharmaceutical preparations described herein may be varied so as to obtain an amount of the active ingredient that is effective to achieve the desired therapeutic response for a particular patient, composition, and mode of administration, without being toxic to the patient. The selected dosage level will depend upon a variety of factors including the activity of the particular compound, the route of administration, the time of administration, the rate of excretion of the particular compound being employed, the duration of the treatment, other drugs, compounds and/or materials used in combination, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start with doses of the compounds described herein at levels lower than that required in order to achieve the desired therapeutic effect and gradually increase the dosage until the desired effect is achieved.

A trimerization inhibitor can be administered alone or in combination with other treatments, therapeutics or agents, either simultaneously or sequentially dependent upon the condition to be treated. Additional agents or therapeutics include, e.g., anti-RANKL agents or antibodies, immune modulators, or anti-resorptive agents, such as progestins, polyphosphonates, bisphosphonate(s), estrogen agonists/antagonists, estrogen, estrogen/progestin combinations, and estrogen derivatives or therapeutics, hormones. Those skilled in the art will recognize that other bone anabolic agents, also referred to as bone mass augmenting agents, may be used in conjunction with a trimerization inhibitor. A bone mass augmenting agent is a compound that augments bone mass to a level that is above the bone fracture threshold as detailed in the World Health Organization Study World Health Organization, “Assessment of Fracture Risk and its Application to Screening for Postmenopausal Osteoporosis” (1994), Report of a WHO Study Group, World Health Organization Technical Series 843. Any prostaglandin, or prostaglandin agonist/antagonist may be used in combination with the compounds of this disclosure. Those skilled in the art will recognize that IGF-1, sodium fluoride, parathyroid hormone (PTH), active fragments of parathyroid hormone, growth hormone or growth hormone secretagogues may also be used.

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound as defined herein may comprise additional component(s) than the ones specifically identified, the additional component(s) not altering the unique characteristic of the disclosure.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

All patent and literature references cited in the present specification are hereby incorporated by reference in their entirety.

The disclosure is further described in the following examples. These examples do not limit the scope of the invention, but merely serve to clarify the invention.

### EXAMPLES

### Example 1

### Generation of a Novel ENU-Induced Mouse Model of Severe Osteopetrosis

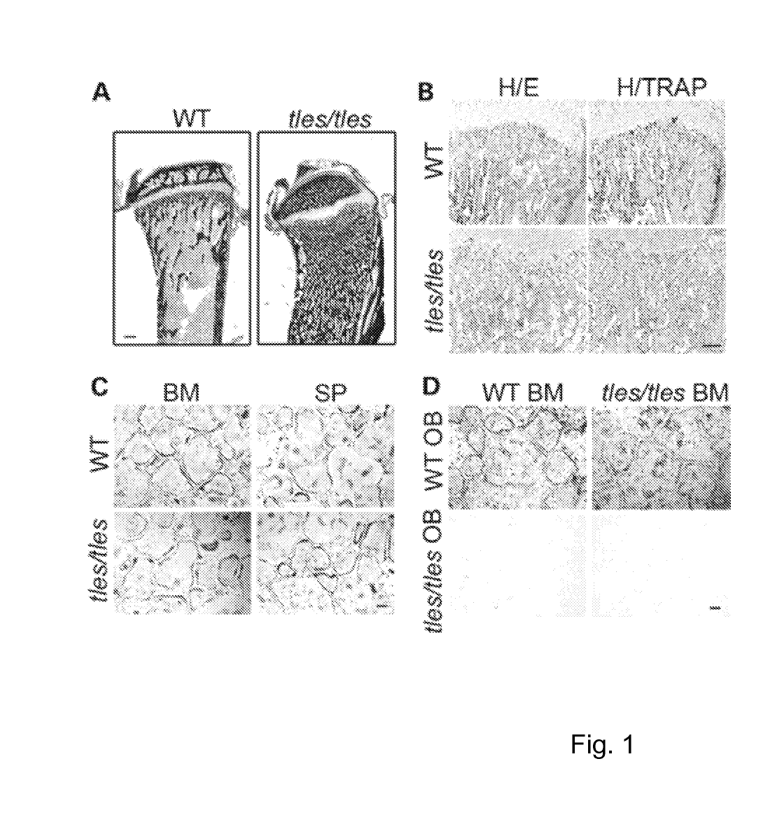

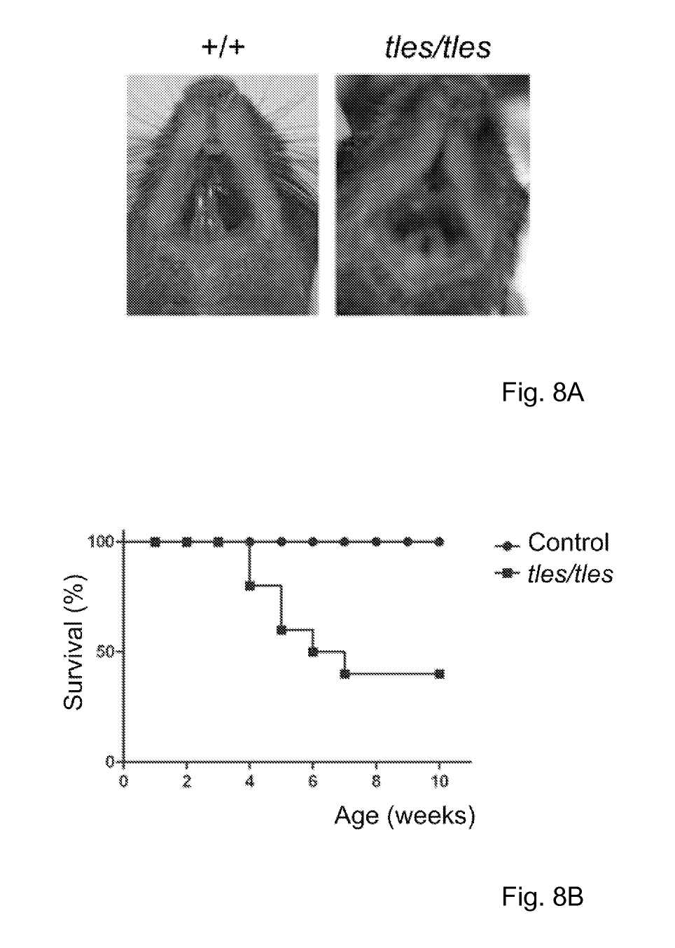

The toothless (tles) phenotype was identified as a recessive trait in which complete failure of tooth eruption was detected in N-ethyl-N-nitrosourea (ENU)-mutagenized G3 mice in both sexes (FIG. 8A). Mutant mice displayed also growth retardation, and lymphoid aberrations characterized by thymic hypoplasia, enlarged spleens, and absence of lymph nodes. Additionally, these mice displayed early lethality, where 60% of the tles/tles mice died by the 7th week of age (FIG. 8B). Since failure of tooth eruption is a typical finding in osteopetrosis, we performed extensive histological analysis of the tibiae and femurs in 4 to 6-week-old tles/tles mice and WT control littermates. Staining of long bones with von Kossa (FIG. 1A), as well as with hematoxylin/eosin (FIG. 1B) revealed severe osteopetrosis in mutant mice whereas staining with tartrate-resistant acid phosphatase (TRAP), an enzyme that is highly expressed in osteoclasts, showed that tles/tles mice completely lacked TRAP-positive (TRAP+) multinucleated osteoclasts (FIG. 1B).

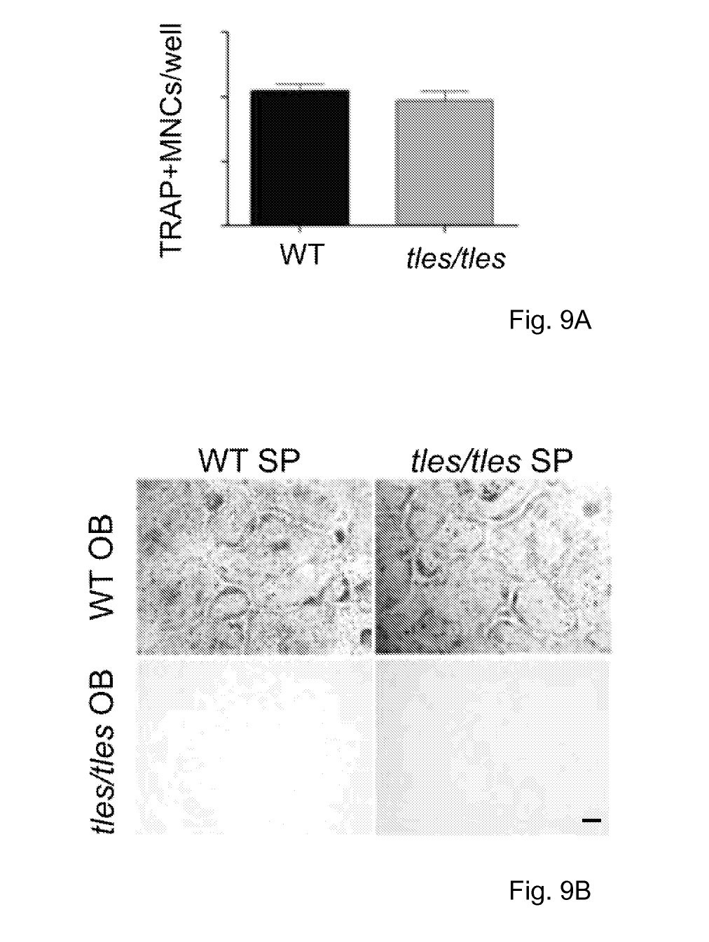

Failure of osteoclast formation can result either from an intrinsic defect in osteoclast differentiation or an impaired cross-talk between osteoclasts and osteoblasts/stromal cells.^{[28, 29]} To discriminate these possibilities we performed ex vivo osteoclastogenesis assays using hematopoietic progenitor cells isolated from bone marrow (BM) or spleens that can differentiate into TRAP+ mature multinucleated osteoclasts in the presence of macrophage colony-stimulating factor (M-CSF) and RANKL.^{[13]} Cultures of BM cells and splenocytes from either WT or tles/tles mice differentiated into TRAP+ multinucleated osteoclasts (FIGS. 1C and 9A), indicating that the intrinsic osteoclast differentiation process is not defective in the tles/tles mice. To determine whether osteoblasts isolated from the tles/tles mice can support osteoclastogenesis, we established ex vivo co-culture assays between primary osteoblast cultures and hematopoietic progenitors from BM or spleens in the presence of 1,25(OH)₂ vitamin D3 and prostaglandin E2 (PGE2).^{[30]} Osteoblasts from WT mice supported osteoclast formation in progenitors isolated either from WT or tles/tles mice, whereas osteoblasts derived from tles/tles mice were inadequate to cross-talk with hematopoietic progenitors and direct their differentiation towards osteoclasts (FIGS. 1D and 9B). These results demonstrate a defective cross-talk between osteoclast precursors and osteoblasts that could be possibly caused by a critical factor missing from the osteoblasts of tles/tles mice.

### Example 2

### tles is a Missense Mutation in the Rankl Gene

The entire genome of 124 F2 animals (62 affected and 62 normal control siblings) was scanned with a collection of 71 polymorphic markers. Initial screening of 20 animals (10 affected and 10 normal siblings) established linkage to distal chromosome 14. Fine mapping of the locus based on 248 meioses, confirmed linkage to 14qD3 at 44cM, between single nucleotide polymorphisms rs13482262 and rs30965774, with a logarithm of odds (LOD) score of 33,8 and a p value equal to 8,80912e⁻⁴² (FIG. 2A).

Screening of the region for candidate genes indicated the presence of the Rankl gene and sequencing of its coding region identified within exon 5 a single base transition of guanine to adenine (GenBank NM_{—}011613.3), resulting in a glycine (G) to arginine (R) substitution at position 278 (G278R) (NP_{—}035743) (FIG. 2B). G278 is located at the hydrophobic F 13-strand of the monomer that is part of the inner A′AHCF β-sheet involved in intersubunit association and trimer assembly.^{[6]} Thus, the G278R substitution is likely to interrupt trimerization of the RANKL monomers due to steric clashes and positive charge introduction (FIGS. 2C and 2D). G278 residue is highly conserved among various TNF superfamily members, including TNF, CD40L, TRAIL, BAFF and APRIL (FIG. 2E).

### Example 3

### Genetic Confirmation of the RANKL G278R Mutation

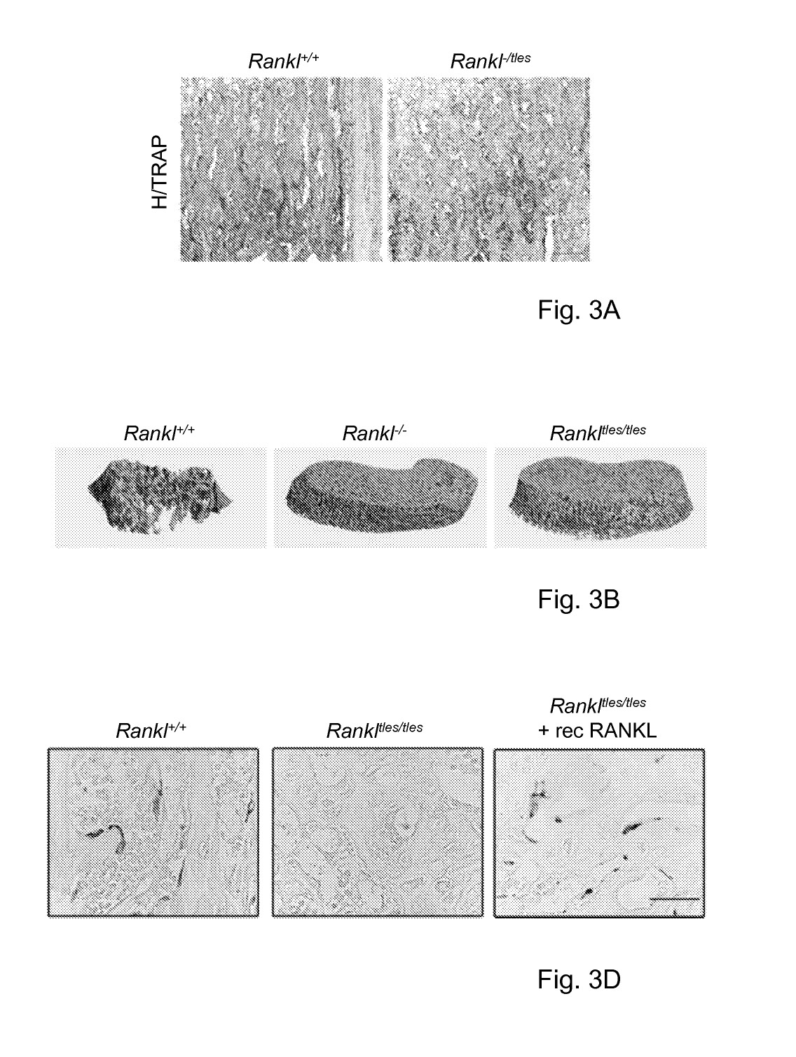

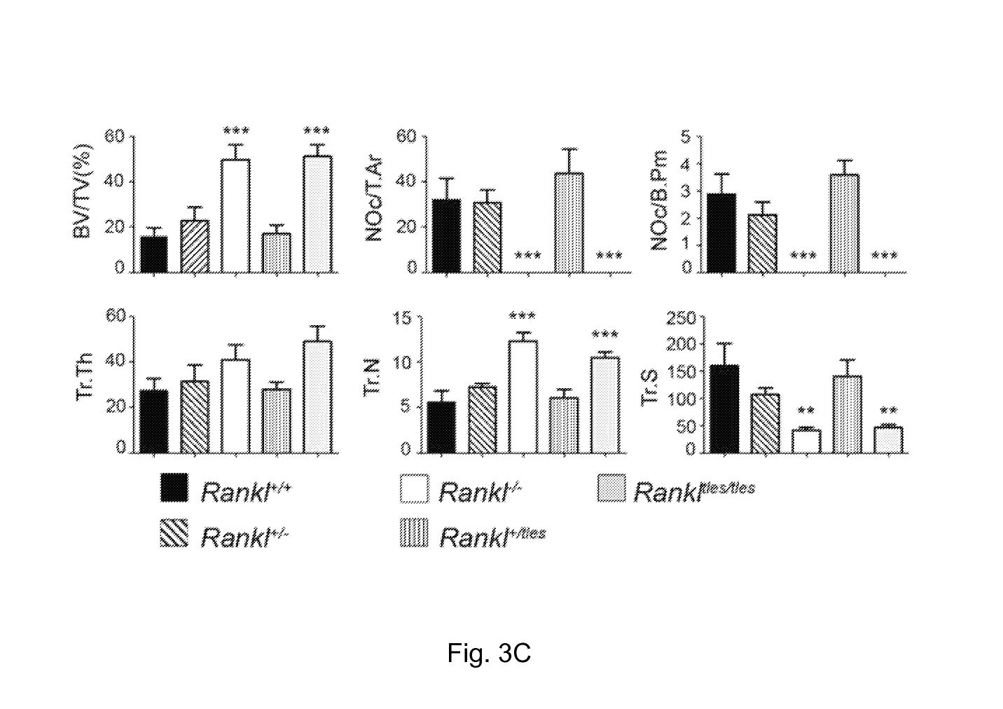

To confirm that the RANKL G278R substitution causes the osteopetrotic phenotype developed in the tles/tles (Rankl^{tles/tles}) mice, we performed genetic complementation by generating Rankl^{-/tles} compound heterozygous mice through intercrosses between heterozygous Rankl^{tles/+} mice and heterozygous Rankl null mice (Rankl^{+/-}).^{[13] Rankl}^{-/tles} mice (n=6) exhibited severe osteopetrosis characterized by failure of tooth eruption, high bone mass and absence of osteoclasts comparable with the phenotype developed in Rankl^{tles/tles} and Rankl^{-/-} mice (FIG. 3A). These results verify that the G to A transition is a loss-of-function mutation that results in severe osteopetrosis in the Rankl^{tles/tles} mice.

Three-dimensional microstructural analyses using high-resolution microcomputed tomography confirmed severe osteopetrosis in Rankl^{tles/tles} mice (FIG. 3B), which was further validated using bone histomorphometric analysis (FIG. 3C). Rankl^{tles/tles} mice develop severe osteopetrosis similarly to Rankl^{-/-} mice, also indicating that the mutant protein is inactive. Interestingly, Rankl^{tles/+} mice are not osteopetrotic and exhibit similar bone parameters, to those of WT control mice and Rankl^{+/-} mice (FIG. 3C).

To verify whether administration of recombinant RANKL restores osteoclast formation in vivo, Rankl^{tles/tles} mice were treated from day 13 of age for a period of 14 days with daily subcutaneous injections of recombinant murine RANKL at 150 μg/kg. A massive formation of TRAP+ cells was identified both in trabecular and cortical bones of RANKL-treated Rankl^{tles/tles} mice indicating that exogenous RANKL efficiently restores osteoclast formation in vivo (FIG. 3D). These results confirm that administration of recombinant RANKL might be considered for the therapy of human RANKL-mediated ARO.^{[27]}

### Example 4

### G278R Impairs RANKL Trimerization and Binding to RANK

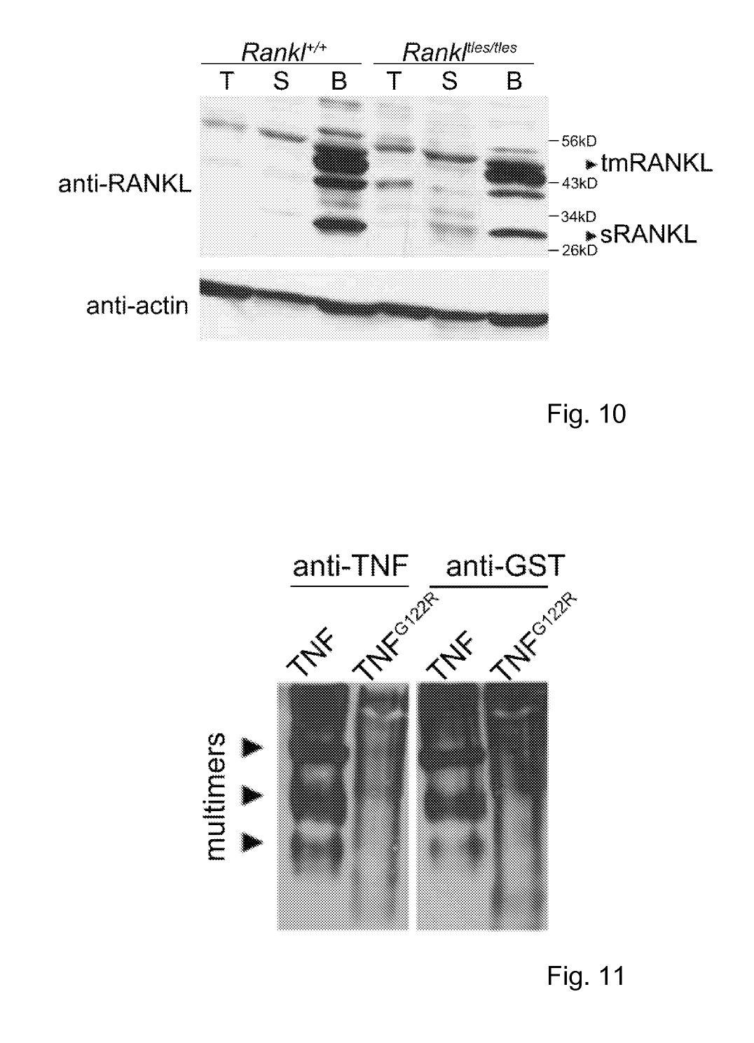

G278R substitution allows normal RANKL gene expression and protein production (FIG. 10). Since G278 resides at the subunit interfaces in the trimer, it may alter trimer formation. To determine whether G278R affects trimer assembly, recombinant soluble WT RANKL and RANKL^{G278R} fused at the N terminus with Glutathione S-Transferase (GST) were produced and characterized biochemically. Previous studies have shown that the GST moiety doesn't impact on RANKL function,^{[31, 32]} whereas it enhances the formation of multimers due to the natural tendency of GST to dimerize. RANKL multimers were detected in WT GST-RANKL, but not in GST-RANKL^{G278R}, using both monoclonal and polyclonal antibodies against murine RANKL or polyclonal antibodies against GST in native polyacrylamide gels (FIG. 4A). Instead, a lower molecular weight band (LB) was detected exclusively in GST-RANKL^{G278R} using polyclonal antibodies against RANKL or GST, which corresponds most probable to GST-RANKL^{G278R} monomers. In addition, both antibodies immunoreacted with high molecular weight GST-RANKL^{G278R} complexes, indicating protein aggregation. The failure of GST-RANKL^{G278R} detection by the monoclonal antibody in native conditions could be explained by the modification of the RANKL^{G278R} structure so that the specific epitopes were either destroyed or masked. However, both GST-RANKL and GST-RANKL^{G278R} were identified by both monoclonal and polyclonal antibodies against RANKL in SDS reduced conditions (FIG. 4A).

The inability of the soluble RANKL^{G278R} protein to form trimers was then verified using chemical cross-linking (FIG. 4B). GST was removed from the RANKL protein with proteolytic cleavage of GST-RANKL bound on glutathione-beads. Even though, soluble WT RANKL was released efficiently from the beads, the majority of the soluble RANKL^{G278R} protein remained bound on the beads after digestion (data not shown). This phenomenon indicates increased hydrophobicity of the RANKL^{G278R} protein due to the formation of hydrophobic protein-protein interactions. Chemical cross-linking of soluble WT RANKL showed a trimer form in addition to a dimer and a monomer form, while without cross-linker only monomers were detected (FIG. 4B).^{[33]} In contrast, cross-linking of the released soluble RANKL^{G278R} protein revealed only the monomer form and a high molecular weight “aggregate” form.

To verify that RANKL^{G278R} cannot form trimers in eukaryotic cells, HEK 293FT cells were transiently transfected with expression vectors of the full-length WT or RANKL^{G278R} fused to FLAG or Myc tag at the C terminus (FIG. 4C). Similar to the analysis of recombinant RANKL proteins, trimer formation was detected only in WT RANKL-Myc but not in RANKL^{G278R}-Myc. Co-transfection of WT RANKL-FLAG with either WT RANKL-Myc or RANKL^{G278R}-Myc revealed the presence of trimer formation only in cells co-expressing both WT forms (FIG. 4C). These results indicate that RANKL^{G278R} not only fails to form trimers but also inhibits WT RANKL trimerization.

Soluble RANKL^{[8, 9]} was detected in supernatants of HEK 293FT cells transfected with WT RANKL-FLAG, WT RANKL-Myc or co-transfected with both WT forms but not in supernatants of cells transfected with RANKL^{G278R}-Myc or co-transfected with WT RANKL-FLAG (FIG. 4D). These results support the failure of trimer assembly in cells expressing RANKL^{G278R} or co-expressing RANKL^{G278R} and WT RANKL, as the specific antibodies recognize epitopes on RANKL trimers that are not formed in the latter cases.

To investigate whether RANKL^{G278R} interacts with WT RANKL, immunoprecipitation was performed. Lysates of HEK 293FT cells transfected with WT RANKL-FLAG in the presence of either WT RANKL-Myc or RANKL^{G278R}-Myc, were immunoprecipitated with an anti-Myc antibody and the immunoprecipitates were assayed for the presence of the FLAG epitope by immunoblot (FIG. 4E). WT RANKL-FLAG co-immunoprecipitated with either WT RANKL-Myc or RANKL^{G278R}-Myc, indicating that WT RANKL interacts with RANKL^{G278R}.

To examine whether RANKL^{G278R} binds to the RANK receptor, serial dilutions of murine RANK-Fc were incubated with immobilized WT GST-RANKL, GST-RANKL^{G278R} or GST (FIG. 4F). RANK-Fc interacted with GST-RANKL in a dose-dependent manner, but not with GST-RANKL^{G278R} or GST. This result shows that the binding affinity of GST-RANKL^{G278R} for RANK-Fc was completely abolished, as a result of its inability to form trimers. Collectively, these results indicate that G278R substitution is critically involved in the abrogation of RANKL trimer formation and subsequently receptor binding.

### Example 5

### RANKL^{G278R} Lacks Biological Activity and Possesses a Dominant Negative Effect

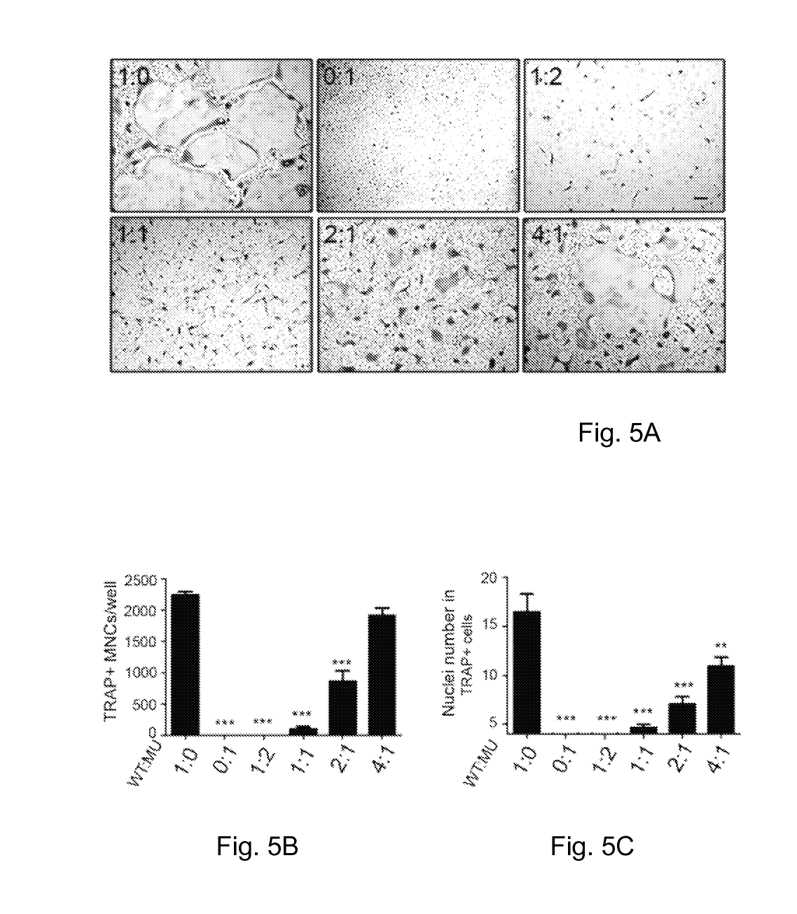

To confirm that RANKL^{G278R} is inactive and to test whether it interferes with the ability of WT RANKL to induce ex vivo osteoclast formation, BM cells were treated with 25 ng/ml M-CSF and 50 ng/ml GST-RANKL for 5 days in the presence or absence of GST-RANKL^{G278R} at different concentrations from 12.5-100 ng/ml. It is prominent that RANKL^{G278R} lacks biological activity as GST-RANKL^{G278R} failed to induce formation of TRAP+ cells (ratio 0:1) (FIGS. 5A-5C). Instead, WT GST-RANKL (ratio 1:0) induced formation of TRAP+ giant osteoclasts (FIGS. 5A-5C). Complete inhibition in the formation of multinucleated TRAP+ osteoclasts was noticed when the concentration of WT GST-RANKL was half of that of GST-RANKL^{G278R} (ratio 1:2). Incubation of WT GST-RANKL with GST-RANKL^{G278R} at equal molar 1:1 concentrations completely impaired the formation of TRAP+ giant multinucleated cells, whereas small size TRAP+ cells with low numbers of nuclei were still formed (FIGS. 5A-5C). However, a small number of TRAP+ giant multinucleated cells was formed at a 2:1 ratio, which were morphologically smaller and exhibited less multinucleation as compared to osteoclasts formed in the presence of WT GST-RANKL exclusively (ratio 1:0). Formation of giant osteoclast-like-cells appeared when WT GST-RANKL was mixed with GST-RANKL^{G278R} at a ratio of 4:1 or higher. Incubation of WT GST-RANKL with GST at similar concentrations (12.5-100 ng/ml), didn't affect the formation of osteoclasts. These results indicate that the RANKL^{G278R} variant lacks biological activity and possesses a dominant negative effect on WT RANKL function.

### Example 6

### G122R Substitution Abrogates TNF Activity

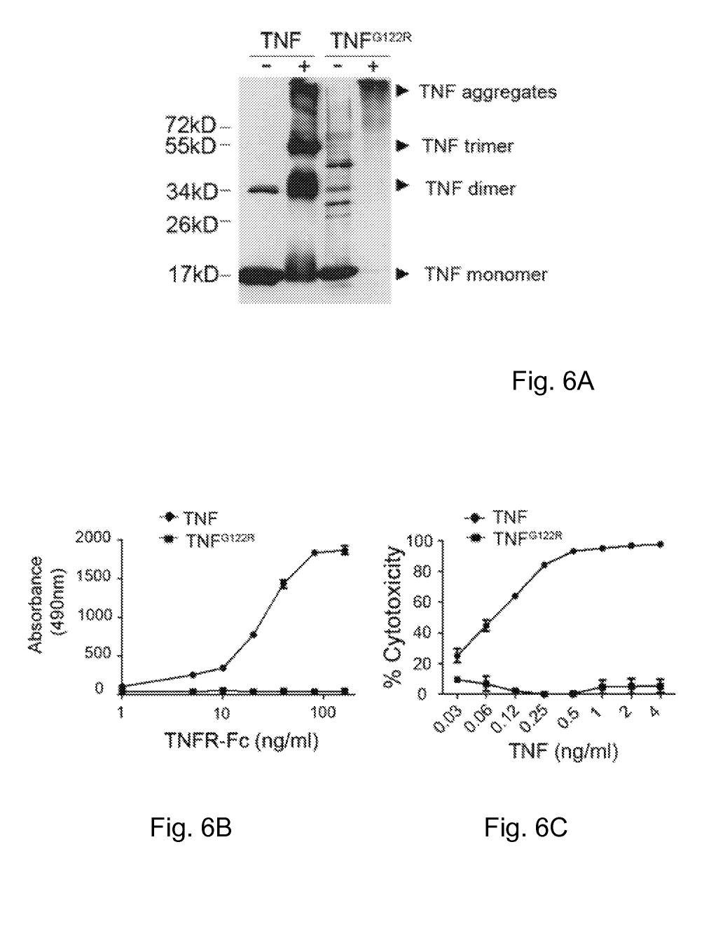

The G278 residue of RANKL is highly conserved among various members of the TNF superfamily (FIG. 2E). Thus, we investigated whether a similar substitution in soluble human TNF, which corresponds to a replacement of glycine with arginine at position 122 (G122R), modifies TNF trimerization and function. TNF multimers were detected in recombinant WT GST-TNF but not in GST-TNF^{G122R} indicating failure of spontaneous trimer assembly. This result was also confirmed by chemical cross-linking (FIG. 6A) of soluble WT TNF or TNF^{G122R} after removal of GST. Similarly to the RANKL^{G278R} variant, G122R substitution in TNF abrogated trimer formation whereas monomers and mainly aggregates were formed instead of trimers, dimers and monomers detected in WT TNF (FIG. 6A).^{[34]}

To examine whether TNF^{G122R} binds to TNF receptor, serial dilutions of human p75TNFR-Fc were incubated with immobilized soluble TNF or TNF^{G122R} (FIG. 6B). p75TNFR-Fc interacted with TNF in a dose-dependent manner, but not with TNF^{G122R} indicating that TNF^{G122R} cannot bind to its receptor. The biological activity of the GST-TNF^{G122R} variant was tested using in vitro cytotoxicity assays. Although recombinant WT GST-TNF induced dose dependent cytotoxicity in L929 cells, GST-TNF^{G122R} was inefficient to induce cytotoxicity not only at similar doses (0.03-4 ng/ml) (FIG. 6C) but also at doses 60 times more concentrated (240 ng/ml). These results indicate that a similar residue substitution in TNF, G122R, is critically involved in the abrogation of TNF trimer assembly, receptor binding and biological activity.

### Example 7

### Small Molecule SPD304 Inhibits RANKL-Induced Osteoclastogenesis

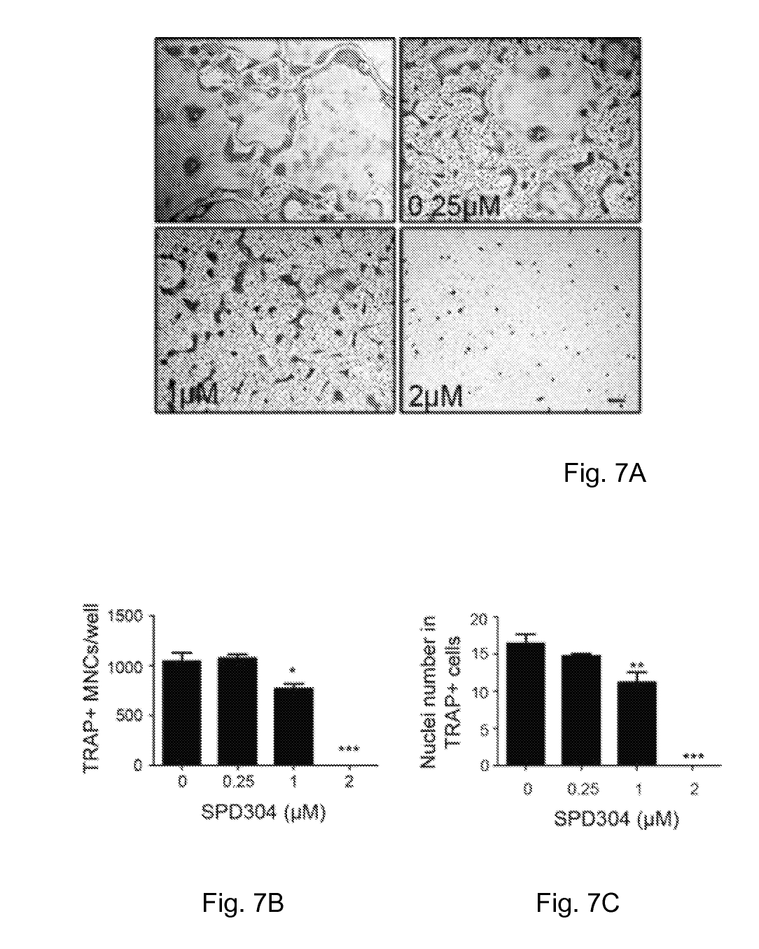

A novel small molecule inhibitor of TNF trimerization, named SPD304, has been recently reported^{[35]} to interact with glycine 122 (G122) that corresponds to G278 in RANKL. To investigate whether SPD304 can also inhibit RANKL-induced osteoclast formation, BM cells were treated with 25 ng/ml M-CSF and 80 ng/ml GST-RANKL in the presence of SPD304 at different concentrations ranging from 0.25 to 2 μM. SPD304 at 1 μM attenuated both the number and the size of TRAP+ multinucleated cells, whereas at 2 μM the formation of multinuclear TRAP+ osteoclast was completely inhibited (FIGS. 7A-7C).

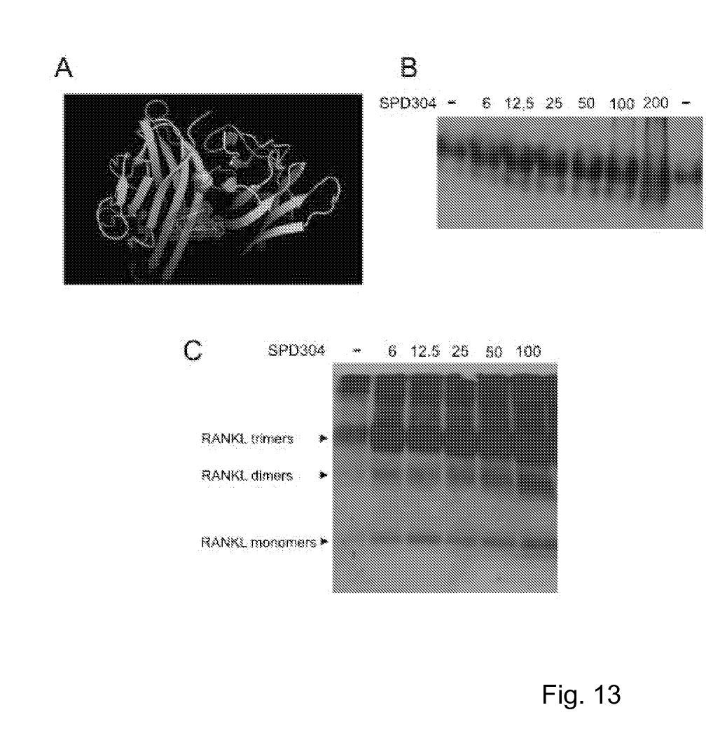

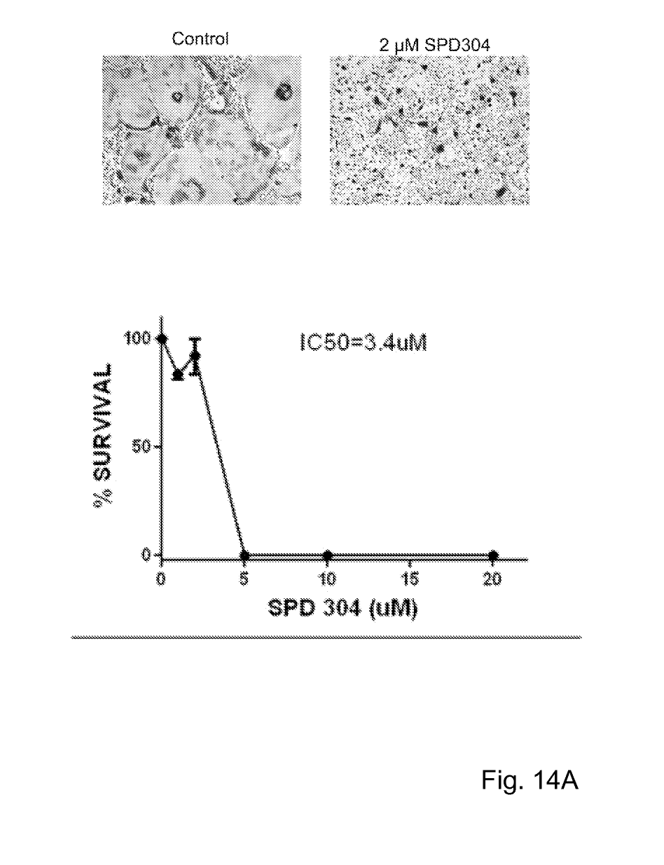

Experimental evidence on the TNF analogue and in silico binding studies on mouse RANKL confirm that the optimal binding position of SPD304, causing trimer inhibition, is located very close (<4 Å) to the G278 mutation position in the structure (FIG. 13, Panel A). To experimentally confirm the interference of the SPD304 with the RANKL structure, soluble mouse RANKL was preincubated with increasing concentrations of SPD304 (6-200 μM) and analyzed in native gels showing the natural conformation of RANKL protein (FIG. 13, Panel B). In the absence of SPD304, soluble RANKL was detected as a single main band whereas a second band of lower molecular weight was also evident in the presence of SPD304. This change of the RANKL conformation appeared even in the lower concentration of SPD304 tested (6 μM) and was more noticeable at 200 μM, indicating a possible release of RANKL dimers and monomers by SPD304. To confirm this, chemical cross-linking experiments were performed in soluble RANKL preincubated with SPD304 at similar concentrations. Indeed, in the presence of SPD304, a dramatic increase of RANKL dimers and monomers was detected indicating disruption of the trimeric RANKL structure (FIG. 13, Panel C). Intriguingly, a significant increase in the intensity of the band corresponding to RANKL trimers was also noticed. This could reflect a possible conformational alteration in the structure of RANKL trimers complexed with SPD304 that lowers the threshold required for the detection of RANKL trimeric molecules by the polyclonal anti-RANKL antibody enabling the detection of more RANKL molecules.

### Example 8

### Inhibition of RANKL Trimerization and Activity by Small Molecules

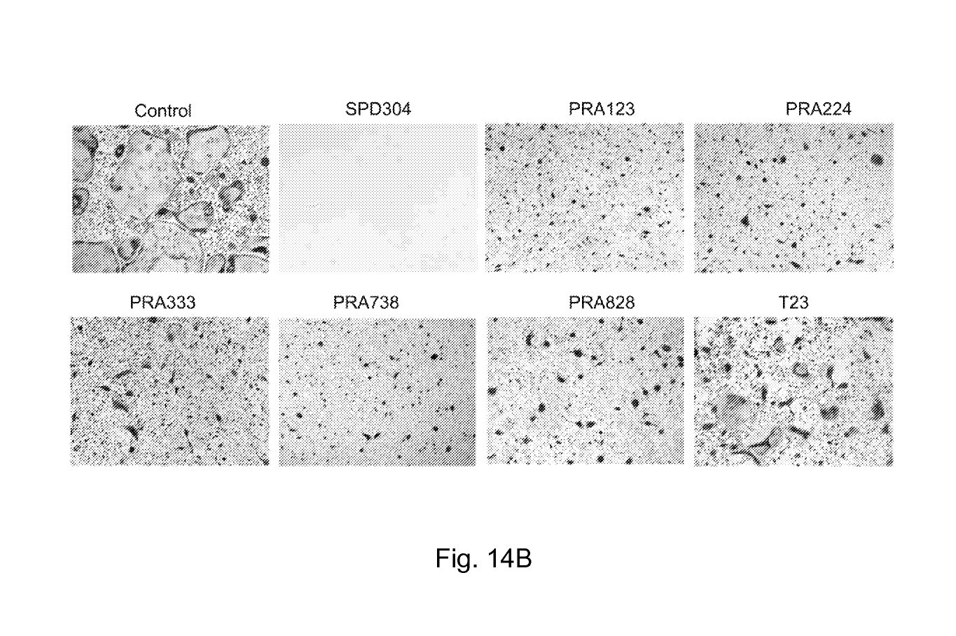

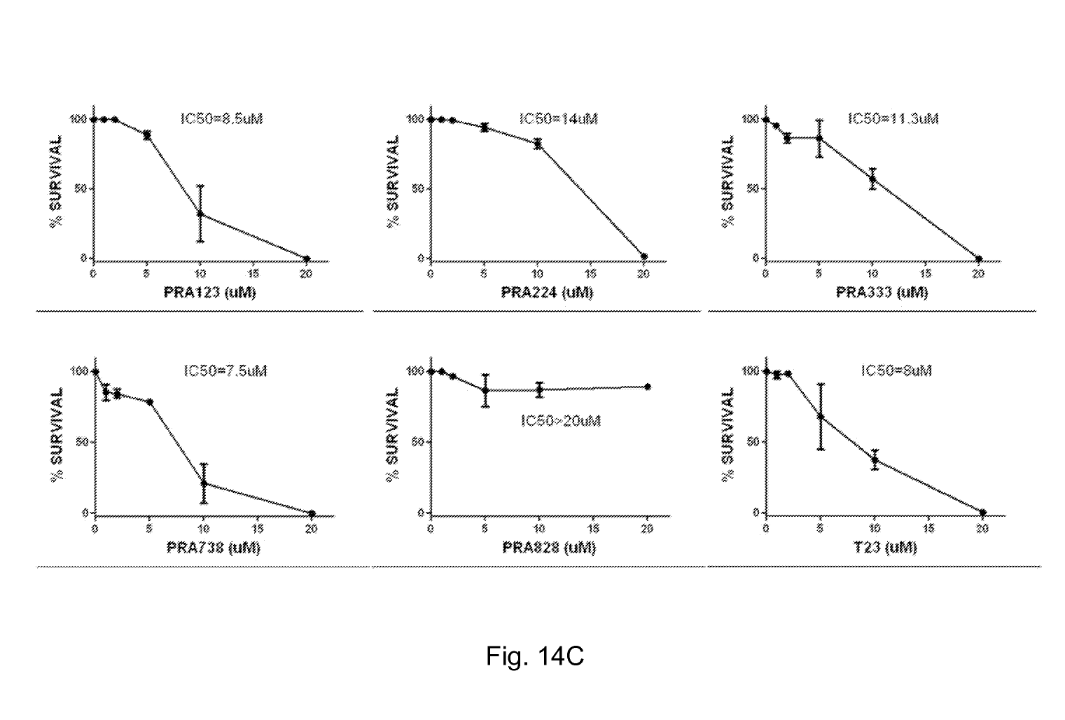

SDP304 at 2 μM is effective in inhibiting human RANKL function (FIG. 14A). However, SPD304 contains a potentially toxic 3-substituted indole moiety that produces reactive intermediates that possibly cause toxicities by covalently binding to nucleophilic residues of protein and/or DNA. In order to evaluate the toxicity induced by SPD304 we established a MTT survival assay in osteoclast precursors and observed that the SPD304 is toxic in concentrations above 5 μM (IC50=3.4 μM) as shown in FIG. 14A. Therefore, we investigated the potential of testing SPD304 derivatives designed to be less toxic with higher specificity for human RANKL. We present here results for the SPD304 derivatives PRA123, PRA224, PRA333, PRA738, and PRA828, that effectively inhibited RANKL activity in osteoclastogenesis assays (FIG. 14B). These small molecules have been also tested as regards their effect on cell toxicity (FIG. 14C). Notably, all these compounds are less toxic compared to SPD304 (IC50>3.4 μM). Among them PRA828 does not affect the survival of osteoclast precursors (IC50>20 μM) and specifically inhibits RANKL-mediated osteoclastogenesis. In addition, the small molecule T23 predicted to interact with the trimerization region also inhibited human RANK activity (FIG. 14B) with IC50=8 μM in cell toxicity (FIG. 14C).

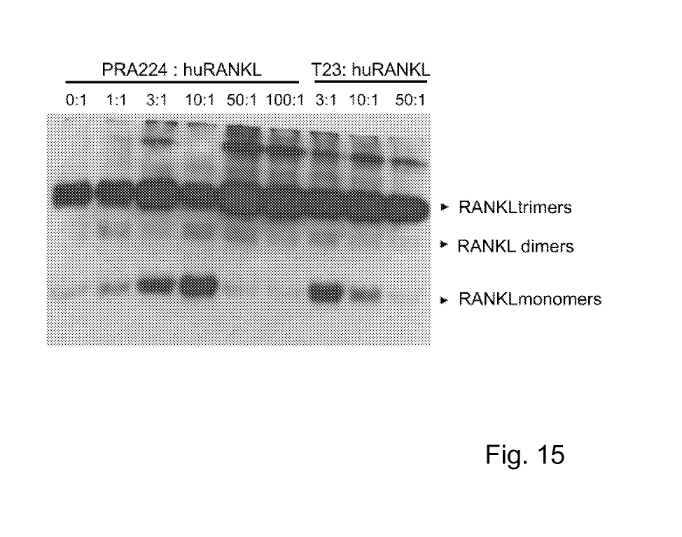

In order to study the effectiveness of these small molecules at the molecular levels, we initially examined the effect of PRA224 and T23 on RANKL trimerization in cross-linking assays and Western blot (FIG. 15). Various molar ratios (from 1:1 to 100:1) between PRA224 and the trimeric form of human RANKL were tested. A gradual increase at the levels of human RANKL monomers was observed at ratios 1:1, 3:1 and 10:1, indicating disruption of the RANKL functional trimers. Similarly, T23 induced an increase of RANKL inactive monomers at the ratios of 3:1 and 10:1. Interestingly, such increase was not observed at higher concentrations of small molecule (50:1, 100:1), indicating that the ratio between the small molecules and the RANKL trimer is critical for the inhibition of trimerization. Collectively, we have identified small molecules targeting the trimerization region of RANKL, which inhibit human RANKL function and display less toxicity compared to SPD304.

### Example 9

### Inhibition of RANKL Trimerization and Activity by Peptides

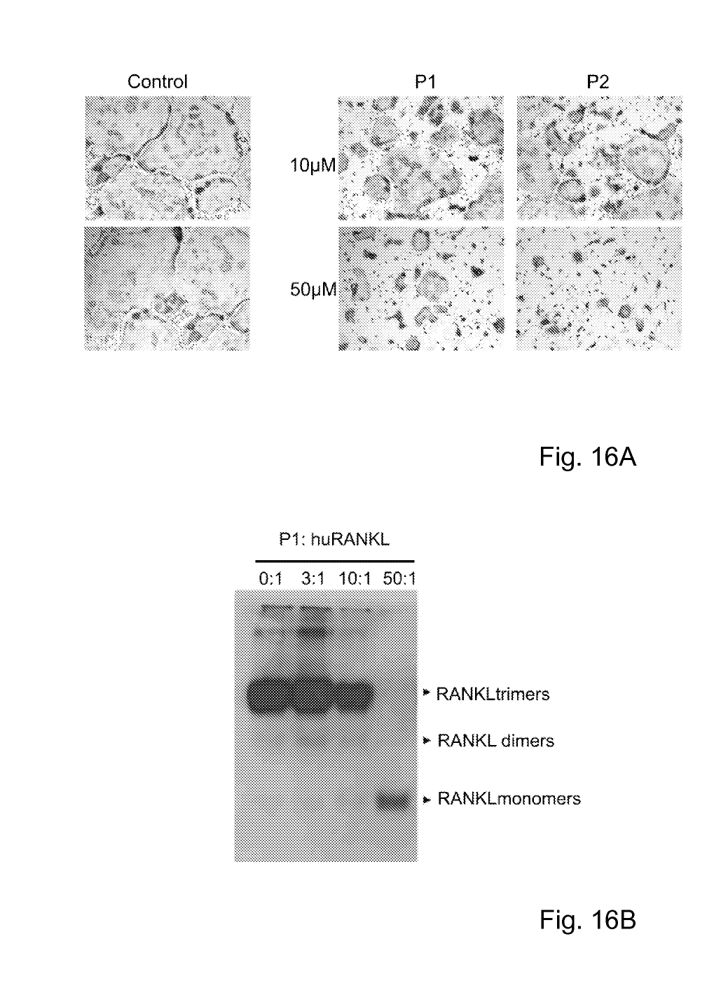

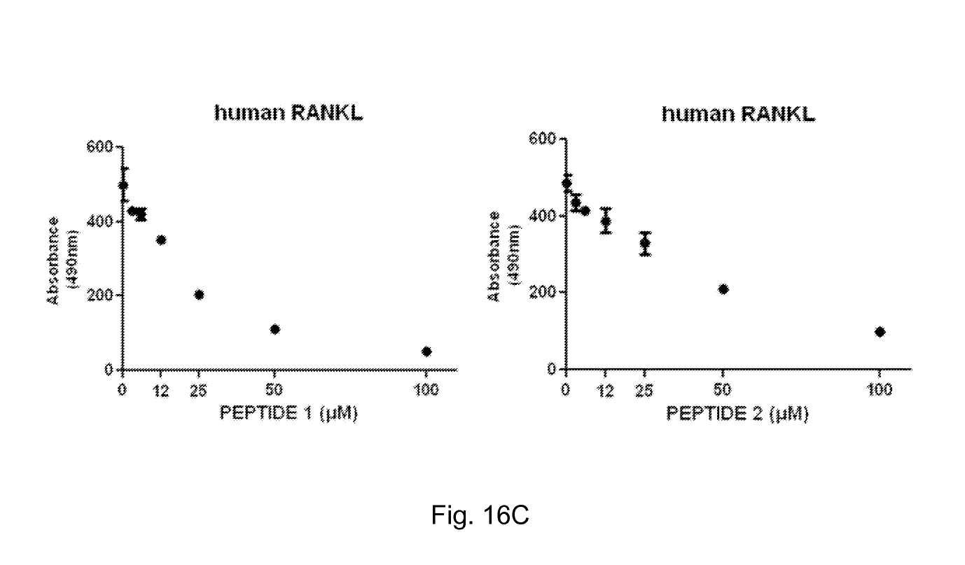

In order to examine whether RANKL trimerization and function is also inhibited by RANKL peptides, we tested the efficacy of 12 mer peptides that correspond to the F β-strand of RANKL. Peptide 1 consists of the wild-type sequence (HFYSINVGGFFK (SEQ ID NO: ______)), whereas peptide 2 contains the glycine to agrinine substitution (HFYSINVGRFFK (SEQ ID NO: ______)). Both peptides inhibited the function of RANKL as detected in RANKL-dependent osteoclastogenesis assays (FIG. 16A). However, the effect of such peptides was not possible to be examined in concentrations higher than 50 μM as in such case there was interference by the increased amounts of DMSO in culture. The effect of the peptide 1 on human RANKL trimerization was tested after cross-linking in Western blotting. A dramatic decrease of the RANKL trimers with a concomitant increase of RANKL inactive monomers was observed at the ratio 50:1 between peptide 1 and RANKL trimers, indicating disruption of RANKL trimers (FIG. 16B). Both peptides were also found to inhibit the binding of human RANKL to its receptor RANK (FIG. 16C).

### Example 10

### Inhibition of TNF-Induced Death

In order to test the ability of the two classes of small molecules to inhibit the function of TNF, one of the most frequently used assays of TNF activity was employed. This exploits the ability of TNF to induce death in the murine fibrosarcoma cell line L929 following sensitization by the transcription inhibitor actinomycin D. If the compounds truly obstruct the activity of TNF at a functional level, they should also prevent it from being cytotoxic in this setting.

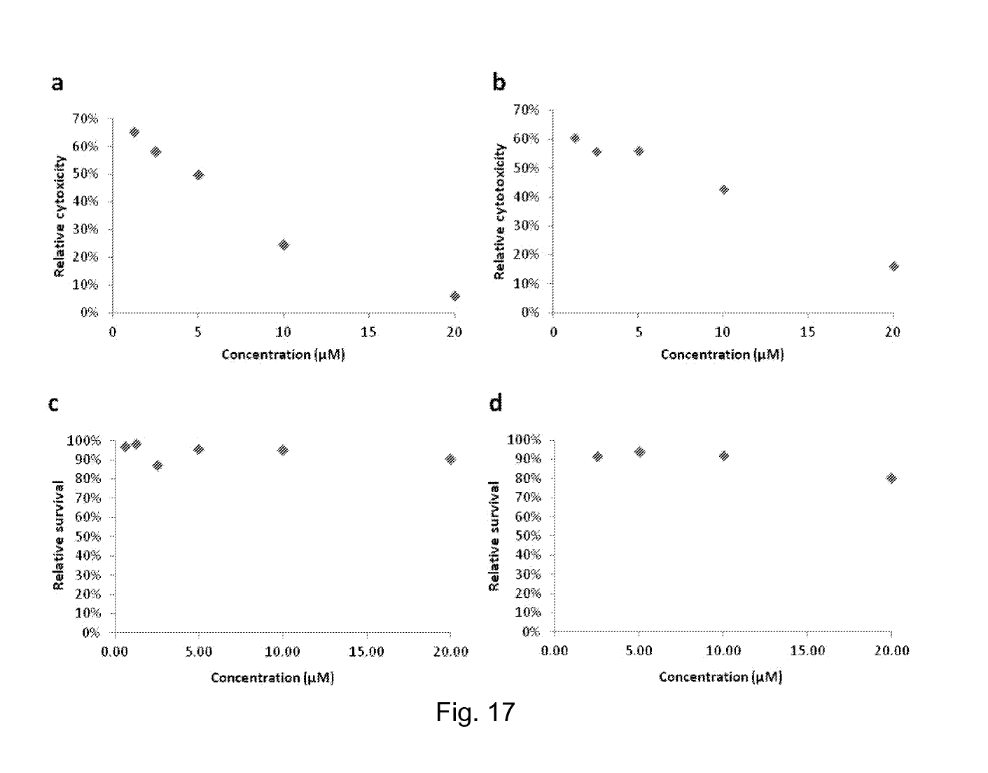

As can be seen in FIG. 17, Panels a and b, both T23 (compound 1) and PRA224 (compound 2) were able to inhibit TNF-driven toxicity in L929 cells. The IC₅₀ values from the respective dose-response experiments were estimated to be less than 10 μM for both compounds. Considering that the read-out of this assay is protection of death, it can also give an indication of the toxicity of the compounds; if they be more toxic than protective, no inhibition would be detected. However, in order to further test any toxicity, the compounds were used in the same concentrations as in the above experiments but with the omission of TNF in order to ascertain whether they exhibit any toxic effects. As is evident in FIG. 17, Panels b and c, both compounds were found to be minimally toxic at least up to a concentration of 20 μM.

### Example 11

### Inhibition of the TNF/TNF-R1 Interaction

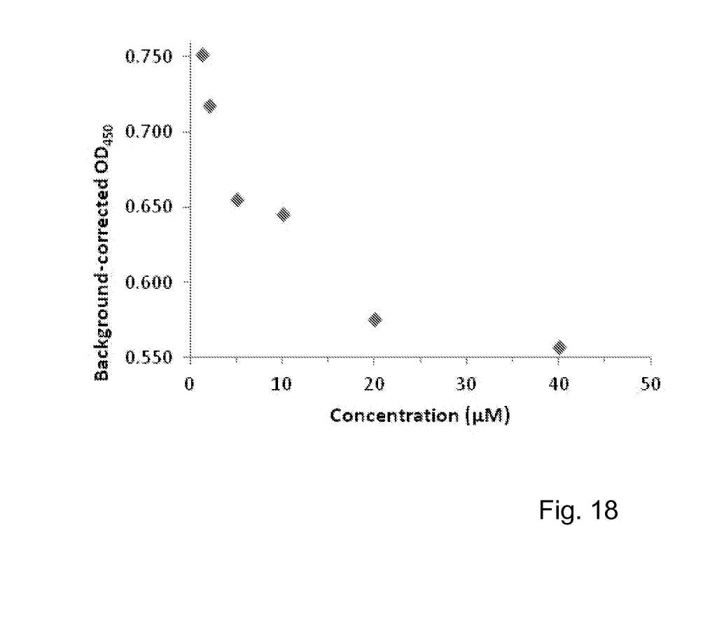

Having established that both compounds (T23 and PRA224) can obstruct the function of TNF, and given that TNF exerts its functions primarily through interacting with the receptor, TNF-R1, a further test was devised to test any effects on this interaction. This test was approached using the ELISA method. Compound 1 (T23) was not found to inhibit the TNF/TNF-R1 interaction in this experimental setting (data not shown). Compound 2 (PRA224) exhibited a pronounced obstruction of this interaction (FIG. 18) with an estimated IC₅₀ of 10 μM.

### Example 12

### Reduction of TNF-Induced MMP Release

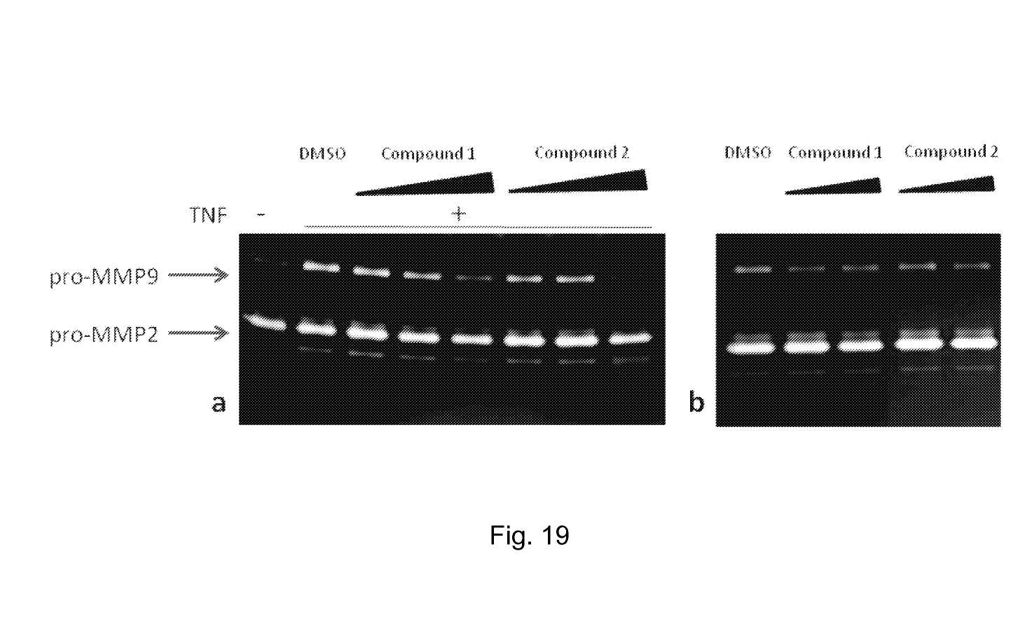

A further line of evidence for the inhibitory capacity of T23 and PRA224 came from taking advantage of the ability of TNF to induce the release of matrix metalloproteinases. It is known that the cellular pathogenic determinant in rheumatoid arthritis, the synovial fibroblast, releases the arthritogenic MMP9 upon stimulation with TNF. It is also known that the human TNF-expressing synovial fibroblast (i.e., isolated from the Tg197 model) releases this MMP naively. As can be seen in FIG. 19, Panel A, both compounds exhibited a dose-dependent reduction in the release of MMP9 in wild-type synovial fibroblasts stimulated by TNF. Notably, a reduction can also be observed in the TNF over-expressing synovial fibroblasts (FIG. 19, Panel B).

### Example 13

### Obstruction of TNF Trimerization

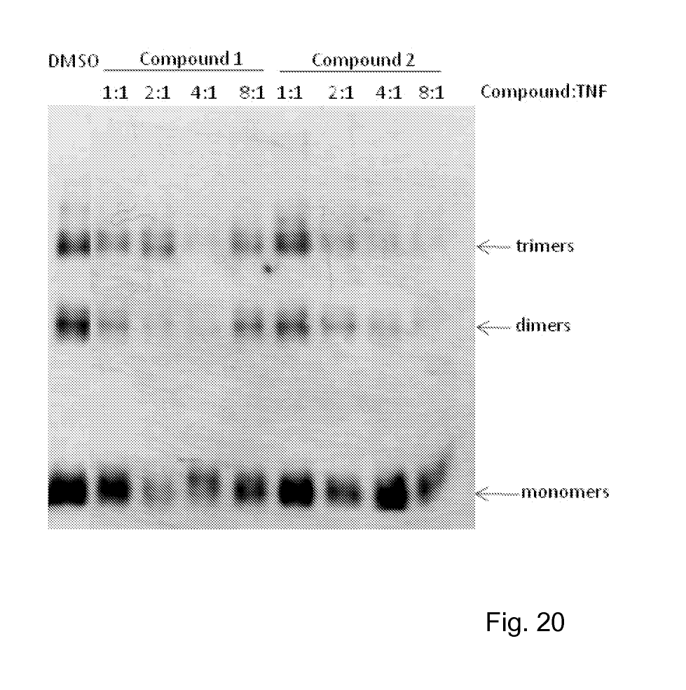

Given that the basis of design of these inhibitors lies in the fact that the functional species of TNF is a trimer, it is anticipated that the most likely mechanism of inhibition characterizing these compounds is a disruption of this trimerization. In order to test this hypothesis, cross-linking experiments were performed so as to detect the various TNF multimers after interaction with the compounds. Preliminary but strong evidence from these experiments indicates that specific molar ratios between inhibitor and TNF can obstruct the formation of trimers, thus resulting in TNF molecules in an inactive, monomeric state (FIG. 20).

### Example 14

### G249R Substitution Abrogates BAFF Activity

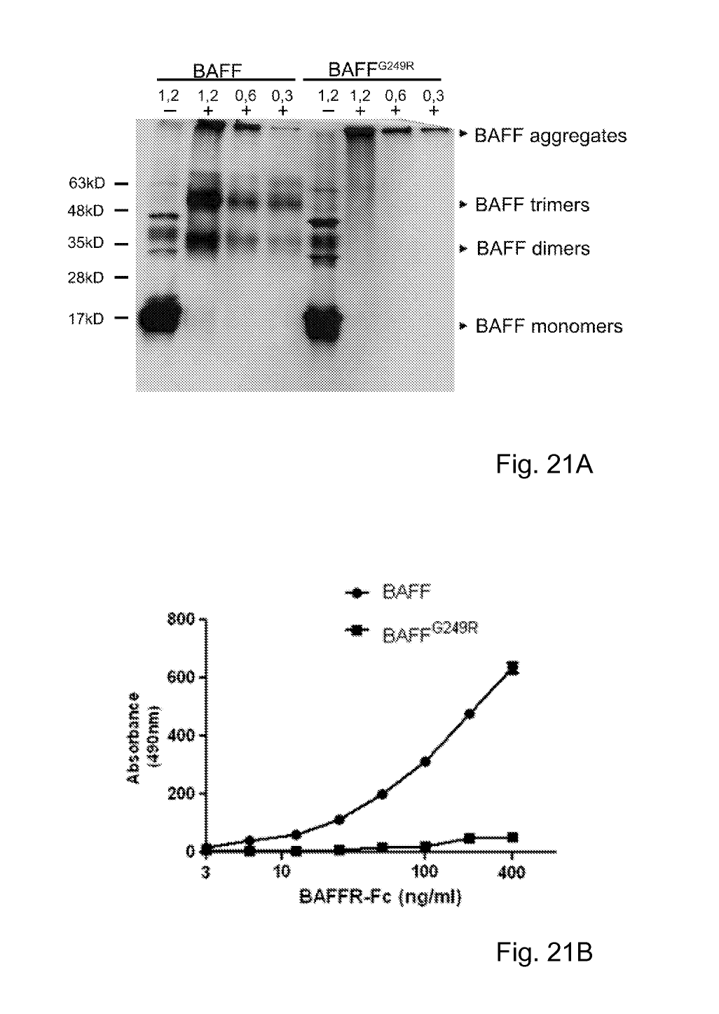

Glycine at codon 278 of mouse RANKL is highly conserved among various members of the TNF superfamily. In order to investigate whether this glycine to arginine substitution is also critical for trimerization in other TNF superfamily members, we reproduced this mutation in human BAFF (BAFF^{G249R}), a cytokine that activates B lymphocytes. Thus, we produced recombinant GST-BAFF in E coli and subsequently the soluble BAFF was released from GST by proteolytic cleavage. Chemical cross-linking of various amounts of soluble BAFF and analysis in Western blot showed the presence of trimers, dimers and monomers in wild-type BAFF but not in BAFF^{G249R} indicating failure of spontaneous trimer assembly in mutant BAFF (FIG. 21A). To examine whether soluble BAFF^{G249R} binds to BAFF receptor (BAFFR), serial dilutions of human BAFFR-Fc were incubated with immobilized soluble BAFF or BAFF^{G249R} (FIG. 21B). BAFFR-Fc interacted with BAFF in a dose-dependent manner, but not with BAFF^{G249R}, indicating that BAFF^{G249R} cannot bind to its receptor. These results indicate that a similar residue substitution in soluble human BAFF, G249R, is critically involved in the abrogation of BAFF trimer assembly, and receptor binding. Thus, substitution of this conserved glycine abrogates trimerization not only in RANKL but also in other TNF superfamily members such as TNF, BAFF and possibly in many more.

### Materials and Methods

Mouse Husbandry

The Rankl^{-/-} mice have been previously reported.^{[13]} DBA/2J mice were purchased from the Jackson Laboratories. Mice were maintained and bred under specific pathogen-free conditions in the animal facility of Biomedical Sciences Research Center (B.S.R.C.) “Alexander Fleming.” All animal procedures were approved and carried out in strict accordance with the guidelines of the Institutional Animal Care and Use Committee of B.S.R.C. “Alexander Fleming” and in accordance to the Hellenic License for Animal Experimentation at the BSRC, “Alexander Fleming” (Prot. No. 3249/18-06-07).

ENU Mutagenesis

G0 males of a mixed C57BL/6Jx129S6 background were treated with ENU (Sigma-Aldrich, Inc.) administered in three weekly doses at 100 mg/kg of body weight.^{[42]} Each G0 mouse was crossed to WT C57BL/6Jx129S6 females to produce G1 males that were further mated with WT females to produce G2 daughters that were subsequently backcrossed with the G1 parent to generate G3 progeny.^{[43]} ENU mutagenesis was performed at B.S.R.C, “Alexander Fleming.”

Mapping and Sequencing

Heterozygous tles/+ animals were outcrossed with DBA/2J mice and the F1 offspring were intercrossed to generate the F2 progeny harboring the recessive ties mutation. F2 progeny were screened for osteopetrosis, and used for genetic analysis. A total of 71 polymorphic markers, including simple sequence length polymorphisms (SSLPs) and single nucleotide polymorphisms (SNPs), were used for genome-wide linkage analysis. SSLPs were resolved on 4% agarose gels whereas SNPs were identified by pyrosequencing using the Pyromark ID instrument (Biotage AB). A standard genome scan was conducted using R/qtl (The R Foundation for Statistical Computing, version 2.8.0).^{[44]} Log likelihood linkage for single-trait analysis was established by non-parametric interval mapping of a binary model (diseased versus healthy control siblings), on 124 F2 animals in total, computed at 1 cM increments over the entire genome. Sequencing was carried out as a service by MWG Biotech AG.

Crystal Structure and Molecular Modeling

The RANKL homotrimer structure was obtained from the Protein Data Bank (PDB) (WorldWideWeb.rcsb.org/pdb/) code 1S55. Molecular models for the G278R mutant homo and heterotrimers were built using Modeller v9.4^{[45]} and tested for packing inconsistencies and atomic clashes using the program QUANTA-CHARM (Molecular Simulations Inc., San Diego, Calif., USA).^{[46]}

Histopathological Analysis

Femurs and tibiae were fixed in 4% PFA for 6 hours, decalcified in 13% EDTA and embedded in paraffin. Sections of 5-μm thickness were stained with hematoxylin/eosin. Osteoclasts were stained for TRAP activity using a leukocyte acid phosphatase (TRAP) kit (Sigma-Aldrich).

Ex Vivo Osteoclast Formation

BM cells were collected after flushing out of femurs and tibiae, subjected to gradient purification using FICOLL-PAQUE™ (GE Healthcare), plated in 24-well plates at a density of 5×10⁵ cells per well and cultured in αMEM medium (GIBCO) containing 10% fetal bovine serum supplemented with 40 ng/ml RANKL (R&D Systems) and 25 ng/ml M-CSF (R&D Systems) for 5 days. Similarly, splenocytes were collected, plated in 24-well plates at a density of 10⁶ cells per well and cultured in the presence of recombinant RANKL and M-CSF for 6 days. GST-RANKL^{G278R} was pre-incubated with WT GST-RANKL at room temperature for 20 min, prior to the stimulation of the BM cell cultures, in order to enable exchange of the RANKL variants and heterotrimer formation. Small molecule SPD304 (Sigma-Aldrich) was pre-incubated with 80 ng/ml GST-RANKL at various concentrations from 0.25 to 2 μM in αMEM medium for 1 hour at room temperature and then added to culture. Osteoclasts were stained for TRAP activity.

Osteoblasts were isolated from calvariae of 10-day-old mice using a sequential collagenase/dispase digestion procedure, were plated in 24-well plates at a density of 4×10⁴ cells per well and cultured overnight in αMEM medium with 10% FBS. BM cells or splenocytes were collected, cultured with 10 ng/ml M-CSF overnight, subjected to gradient centrifugation and co-cultured with osteoblasts at a density of 5×10⁵ (BM cells) and 2×10⁶ (splenocytes) in αMEM medium supplemented with 1.25 (OH)₂ vitamin D3 (10 nM) and PGE2 (1 μM) for 6 days.

Bone Histomorphometry

Left femurs were fixed in 4% formalin and embedded in methylmethacrylate resin (Technovit; Heraeus Kulzer, Wehrheim, Germany) using standard procedures. 4 μm thick sections were prepared with a Jung microtome (Jung, Heidelberg, Germany), and stained with von Kossa stain and toluidine blue. Standard bone histomorphometric measures were analyzed using a Zeiss Axioskop 2 microscope (Zeiss, Marburg, Germany) equipped with an Osteomeasure image analysis system.

MicroCT Imaging

MicroCT images were acquired on a vivaCT40 (Scanco Medical, Bassersdorf, Switzerland). The scanner generates a cone beam at 5-mm spot size and operates at 50 keV. Images of femurs from WT, Rankl^{-/-}, Rankl^{-+/-}, Rankl^{tles/tles} and Rankl^{+/tles} mice were acquired.

Quantification of Soluble RANKL

The levels of soluble mouse RANKL were quantitated using a commercial ELISA kit (R&D).

Expression and Purification of GST-RANKL and GST-TNF

The extracellular domains of RANKL, RANKL^{G278R}, TNF, and TNF^{G122R} were expressed in Escherichia coli as a GST-fusion protein. Briefly, a cDNA encoding the core ectodomain of murine RANKL residues 158-316, with or without the G278R substitution, was cloned into pGEX-6P-1 (GE Healthcare Life Sciences) downstream of GST. For the generation of recombinant GST-TNF, a cDNA encoding the extracellular domain of human TNF from valine 77 to leucine 233 was also cloned into pGEX-6P-1. The G122R substitution was introduced by a two-step overlapping PCR approach. Following IPTG-mediated (100 μM) induction of protein expression, BL21 cells were lysed by sonication, and incubated with glutathione-sepharose beads. The GST-fused proteins were released from the affinity matrix by competitive elution with 50 mM glutathione (Sigma-Aldrich).

Purification of Soluble RANKL and TNF

After capture of GST-RANKL or GST-TNF on glutathione beads, soluble RANKL or TNF were eluted by cleavage of beads with PRESCISSION® Protease (GE healthcare) for overnight at 4° C.

Protein Cross-Linking Assay

The chemical cross-linking reagent disuccinimidyl suberate (DSS, Sigma) was used to examine the trimeric property of RANKL and TNF.^{[33]} 50 mM of DSS was prepared as a stock solution in dimethyl sulfoxide (DMSO). RANKL or TNF proteins at a final concentration of 0.1 mM in PBS buffer (pH 7.5) were mixed with 1 mM DSS (the molar ratio of DSS is 10:1). The cross-linking reactions were carried out for 1 hour at room temperature and terminated with 50 mM Tris (pH 7.5) for 30 minutes. Proteins from reaction mixtures were separated on 12% SDS-PAGE, followed by staining with Coomassie blue R-250 or proceeded in Western blot.

Generation of C-Terminus Tagged Full-Length WT and RANKL^{G278R}

The full-length mouse WT or RANKL^{G278R} cDNA constructs encoded residues 1-316 without stop codon. A Myc-tagged RANKL expression vector was constructed by inserting full-length RANKL into the pcDNA3.1/myc-His A MCS vector (Invitrogen). FLAG-tagged RANKL was created by subcloning full-length RANKL into the p3XFLAG-CMV-14 expression vector (Sigma-Aldrich).

Transient 293 Transfection Assays

HEK 293FT cells were transfected with 1 μg plasmid DNA using TransIt-293 transfection reagent (Mirus, Madison, Wis.). After 48 hours, transfected cells were harvested in PBS and the half quantity was diluted in equal volume of 2× Laemmli sample buffer, and analyzed in 12% acrylamide denatured gels. The remaining cells were lysed by sonication, centrifuged, and analyzed in 8% native acrylamide gels.

Western Blot

Recombinant proteins or lysates were resolved either on 8% native acrylamide gels or 12% SDS denatured acrylamide gels. RANKL was detected by Western blotting using either a monoclonal (clone IK22/5, eBioscience) or a polyclonal (R&D Systems) anti-RANKL antibody, whereas for GST detection a rabbit polyclonal anti-GST antibody was used. Human TNF was detected using a rabbit polyclonal anti-TNF antibody provided by Prof. Wim Buurman (Maastricht University). Moreover, antibodies against Myc (rabbit polyclonal, Santa Cruz Biotechnology), FLAG (M2, Sigma) and actin (goat polyclonal, Santa Cruz Biotechnology) were also used.

Immunoprecipitation

HEK 293FT cells were harvested 48 h after transient transfection, lysed and incubated with an anti-Myc antibody. Anti-Myc immunocomplexes were precipitated with protein A/G Sepharose (Santa Cruz Biotechnology). Protein complexes were resolved by SDS-PAGE, and immunoblotted with an anti-FLAG antibody as described previously.

Binding Assay of GST-RANKL^{G278R} to RANK

Nunc plates were coated with recombinant WT GST-RANKL, GST-RANKL^{G278R} or GST at 3 μg/ml and after blocking with 1% BSA, were incubated with increasing amount of recombinant mouse RANK-Fc (R&D systems). RANK binding was detected with a phycoerythrin (PE) conjugated goat anti-human IgG (Fc) (SouthernBiotech, Birmingham, USA) that was measured (539-573 nm) with the fluorescent plate reader TECAN infinite M200.

Binding Assay of TNF^{G122R} to TNFR

Nunc plates were coated with recombinant soluble TNF or TNF^{G122R} at 3 μg/ml and incubated with increasing amount of recombinant human p75TNFR-Fc (Wyeth). TNFR binding was detected with a horseradish peroxidase (HRP) conjugated goat anti-human IgG (Fc) (SouthernBiotech, Birmingham, USA) using o-phenylenediamine (OPD) substrate (Thermo Scientific Pierce) that was measured at 490 nm.

In Vivo Administration of Soluble RANKL

Recombinant soluble RANKL was produced after digest of the GST-RANKL protein with PRESCISSION® protease (GE Healthcare) for the removal of GST. Mice were treated from day 13 of age for a period of 14 days with subcutaneous injections of 150 μg/kg soluble RANKL.

Statistical Analysis

Statistical analysis was performed on Prism software, using one-way ANOVA with Tukey's Multiple Comparison Test. All values are reported as the mean±standard error of the mean (SEM). All P values below 0.05 were considered significant.

Small Molecules

All compounds were dissolved and stored in DMSO. All pre-incubations with recombinant human TNF were carried out for 30 minutes at room temperature, whereas for human RANKL preincubations were performed at 37° C. for 1 hour.

Based on the crystal structure of RANKL and its predicted interactions with SPD304, novel SPD304 derivatives such as PRA123, PRA224, PRA333, PRA738, and PRA828 were designed to inhibit RANKL activity by targeting its trimerization.

The synthesis of these novel compounds was performed using standard methods known to one of skill in the art. In an exemplary embodiment, the SPD304 derivatives can be prepared as described below. It is clear to a skilled person that other methods may also be used. It will also be appreciated by persons skilled in the art that within certain of the processes described herein, the order of the synthetic steps employed can be varied and will depend inter alia on factors such as the nature of functional groups present in a particular substrate and the protecting group strategy (if any) to be adopted. Clearly, such factors will also influence the choice of reagent to be used in the synthetic steps.

The method of preparation includes reacting aldehydes or acids, which can be same or different, containing saturated or unsaturated ring systems, optionally substituted and optionally containing heteroatoms, with substituted or unsubstituted diamines to form amines or amides respectively. This can be accomplished in a single reaction or in several steps including, but not limited to, steps such as Schiff's base formation, reduction, and reductive amination, as shown in the schemes below.

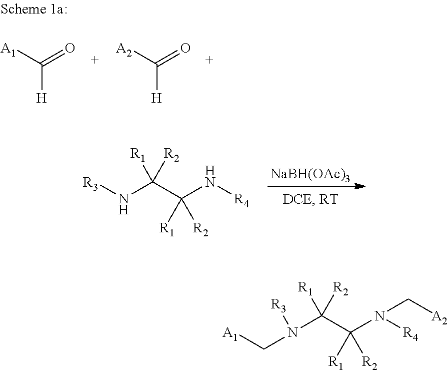

wherein:
A1 and A2 are independently a substituted or unsubstituted phenyl group or a substituted or unsubstituted heterocyclic system, as defined herein above;
R1 and R2 are independently, hydrogen or (C₁-C₄)-alkyl group;
R3 and R4 are independently, hydrogen or (C₁-C₄)-alkyl group; and
R3 and R4 can optionally form a ring system.

The process of Scheme 1a is analogous to the process disclosed in U.S. Pat. No. 6,344,334 and Tetrahedron Lett. 37:7193-7196 (1996).

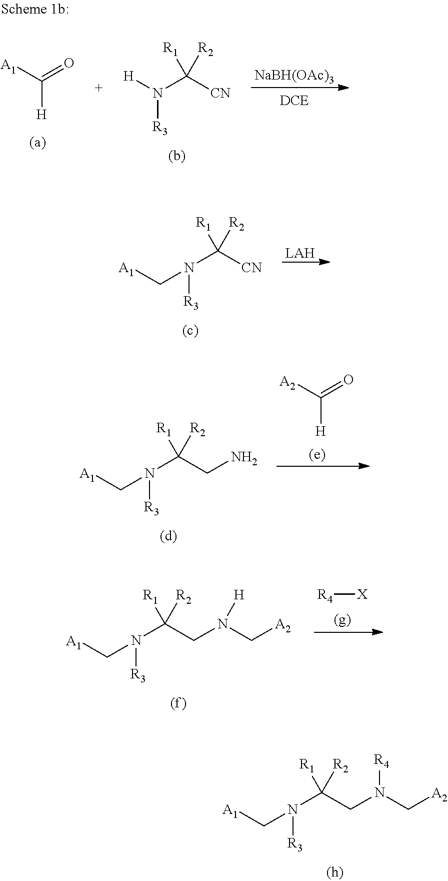

wherein:
A1 and A2 are independently a substituted or unsubstituted phenyl group or a substituted or unsubstituted heterocyclic system, as defined herein above;
R1 and R2 are independently, hydrogen or (C₁-C₄)-alkyl group;
R3 and R4 are independently, hydrogen or (C₁-C₄)-alkyl group; and
R3 and R4 can optionally form a ring system.

In Scheme 1b, reductive amination of an aromatic aldehyde (a) with amino nitrile (b) compound provides a substituted nitrile intermediate (c). The reducing agent used can be selected from, for example, sodium triacetoxy borohydride and sodium cyanoborohydride in solvents such as DCE, THF, acetonitrile and dioxane. In an embodiment, sodium triacetoxy borohydride is used as reducing agent in THF as solvent. The temperature used is 20-40<0>C, for example, ambient temperature (25<0>C). 1.0 equivalent of the intermediate (c) is taken in a suitable solvent such as ether, THF or dioxane at O<0>C and treated with LAH (Lithium aluminium hydride) (0.5 to 2.5 equivalent) to obtain an amino intermediate (d). In an embodiment, the solvent used is THF. The amino intermediate (d) is then reacted with an aldehyde (e) to give a compound (f) (intermediate/product) by reductive amination, which can be N-alkylated using suitable alkyl halide (g) in solvent such as DMF or acetone, in presence of a base such as pyridine, triethylamine, sodium hydride, sodium carbonate or potassium carbonate to give the desired product (h).

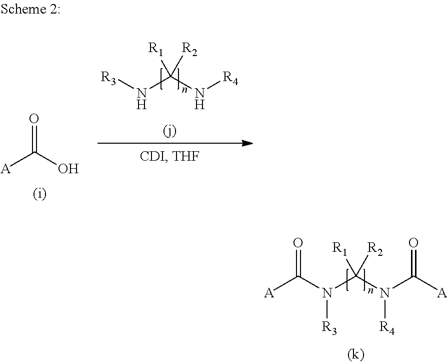

wherein:
A1 and A2 are independently a substituted or unsubstituted phenyl group or a substituted or unsubstituted heterocyclic system, as defined herein above;
n is an integer from 2-4;
R1 and R2 are independently, hydrogen or (C₁-C₄)-alkyl group;
R3 and R4 are independently, hydrogen or (C₁-C₄)-alkyl group; and
R3 and R4 can optionally form a ring system.

In Scheme 2, an aromatic acid (i) is treated with a diamine (j) in presence of a coupling agent in a suitable solvent to obtain compound (k). The coupling agent used can be, for example, CDI (1,1′-Carbonyldiimidazole), DCC (1,3-Dicyclohexylcarbodiimide), EDC (1-(3-Dimethylaminopropyl)-3-ethylcarbodiimide hydrochloride), chloro-dipyrrolidinocarbenium tetrafluoroborate, PyBOP (Benzotriazol-1-yl-oxy-thspyrrolidinophosphonium hexafluorophosphate), HOBT (1-Hydroxybenzotriazole), or DIPEA (N,N-Diisopropylethylamine). In an embodiment, CDI is used as the coupling agent. The solvent used can be, for example, THF, ether, dioxane, or DMF. In an embodiment, the solvent used is THF. The temperature used is 20-40<0>C, for example, ambient temperature (25<0>C). The time required for the completion of the reaction is 3-10 hours. In an embodiment, the reaction is mostly completed in 6 hours. The resulting product is purified by various methods, which optionally include free base isolation or salt formation. Normal phase or reversed phase silica gel chromatography or precipitation techniques are used wherever required.

The reagents, reactants and intermediates used in the present processes are either commercially available or can be prepared according to standard literature procedures known in the art.

Compound 1 of FIG. 23 (T23) was identified in a multi-step in silico approach, including computational molecular docking studies. A chemical library of more than 14,000 small molecules was screened to identify molecules with suitable properties that are predicted to interact with the TNF-alpha dimer. T23 was identified and its ability to act as a trimerization inhibitor was confirmed in vivo. The PubChem database was used to identify chemicals having a similar 3D structure as T23. The functional derivatives listed in FIG. 23 are predicted to interact with residues from TNF superfamily polypeptides. T23 and its functional derivatives are commercially available or can be prepared according to standard literature procedures known in the art.

RANKL Peptides

12 mer peptides corresponding to the F-beta strand of RANKL were synthesized by JPT Peptide Technologies GmbH. The sequence of the wild-type peptide (peptide 1) was HFYSINVGGFFK (SEQ ID NO:4), whereas the sequence of the peptide containing the glycine to arginine substitution (peptide 2) was HFYSINVGGFFK (SEQ ID NO:5). Peptides were dissolved and stored in 100% DMSO. The purity of the peptides was above 90%.

Expression and Purification of Soluble Human RANKL

The extracellular domain of human RANKL was expressed in Escherichia coli as a GST-fusion protein as previously described (Douni et al., 2012). Briefly, a cDNA encoding the core ectodomain of human RANKL residues 143-317 (20 kD), was cloned into pGEX-6P-1 (GE Healthcare Life Sciences) downstream of GST. Following IPTG-mediated (100 μM) induction of protein expression, BL21 cells were lysed by sonication, and incubated with glutathione-sepharose beads. After capture of GST-RANKL on glutathione beads, soluble human RANKL were eluted by cleavage of beads with PRESCISSION® Protease (GE healthcare) for overnight at 4° C.

RANKL Cross-Linking and Western

The chemical cross-linking reagent disuccinimidyl suberate (DSS, Sigma) was used to examine the effect of potent RANKL inhibitors (small molecules, peptides) in the trimerization of human RANKL (Douni et al., 2012). Recombinant soluble human RANKL (prepared in our laboratory) was pre-incubated with increasing amounts of inhibitors at various ratios for 1 hour at 37° C. Such complexes were mixed with 1 mM DSS (the molar ratio of DSS is 10:1). The cross-linking reactions were carried out for 1 hour at room temperature and terminated with 50 mM Tris (pH 7.5) for 30 minutes. Cross-linked soluble human RANKL protein was separated on 12% SDS-PAGE, and was detected using a polyclonal goat anti-RANKL antibody (R&D Systems) in Western blotting.

Rankl/Rank ELISA

Nunc plates were coated with 100 μl of 250 ng/ml recombinant soluble human RANK-Fc (R&D Systems) overnight. Recombinant soluble human RANKL at 200 ng/ml was pre-incubated with increasing amounts of peptides (3-100 μM) for 1 hour at 37° C. and was added in the RANK-coated wells. RANKL binding was detected with a polyclonal goat anti-RANKL antibody (R&D Systems), followed by a horseradish peroxidase (HRP) conjugated horse anti-goat IgG (Vector) using o-phenylenediamine (OPD) substrate (Thermo Scientific Pierce) that was measured at 490 nm.

RANKL-Mediated Osteoclastogenesis Assays

BM cells were collected after flushing out of femurs and tibiae, subjected to gradient purification using FICOLL-PAQUE™ (GE Healthcare), plated in 96-well plates at a density of 6×10⁴ cells per well and cultured in αMEM medium (GIBCO) containing 10% fetal bovine serum supplemented with 50 ng/ml human RANKL (Peprotech) and 25 ng/ml M-CSF (R&D Systems) for 5 days. RANKL was pre-incubated with inhibitors at 37° C. for 1 hour, prior to the stimulation of the BM cell cultures, in order to enable potent interactions. Osteoclasts were stained for TRAP activity (Sigma).

TNF-Induced L929 Cytotoxicity Assay

L929 cells were seeded onto a 96-well plate (3×10⁴ cells/well). On the following day, cells were treated with 0.25 ng/ml human TNF and 2 μg/ml actinomycin D. After 18-24 hours, dead cells were removed by washing with PBS, remaining live cells were fixed with methanol, stained with crystal violet and quantified spectrophotometrically at 570 nm after solubilization of the stain using acetic acid.

TNF/TNF-R1 ELISA

96-well plates were coated with 0.1 μg/ml recombinant soluble human TNR-R1 in PBS over-night at 4° C. Following four washes with PBS containing 0.05% TWEEN®-20, blocking was carried out using 1% BSA in PBS. 0.025 μg/ml recombinant human TNF in PBS was added and the plates were incubated for 1 hour at room temperature. After another round of washes, plates were incubated with a 1:5000 dilution of an anti-TNF antibody conjugated with HRP for 1 hour at room temperature. After a final round of washes, the signal was developed using TMB and measured spectrophoto-metrically at 450 nm.

Gelatin Zymography

For gelatin zymography experiments, serum-free supernatants were collected from serum-starved cells usually after 24 hours of stimulation. Following non-reducing SDS-PAGE in gels containing 1 mg/ml gelatin, these were incubated for 18 hours in developing buffer (50 mM Tris-HCl, pH 7.5; 5 mM CaCl₂; 0.02% NaN₃; 1 μM ZnCl₂) at 37° C. Finally, gels were stained with 0.5% Coomassie Brilliant Blue 8250 in 45% methanol/10% acetic acid and de-stained with 50% methanol/10% acetic acid.

TNF Cross-Linking Experiments

100 ng of recombinant human TNF was cross-linked using 4.8 mM BS3 for 45 min at room temperature. The reaction was stopped by adding 1/10th volume of 1 M Tris-HCl, pH 7.5. Samples were then subjected to SDS-PAGE and Western blotting using an anti-TNF antibody.

Expression and Purification of Soluble BAFF

The extracellular domains of BAFF and BAFF^{G249R} were expressed in E. coli as a GST-fusion protein. Briefly, a cDNA encoding the core ectodomain of human BAFF residues 134-285 (17.5 kD), with or without the G249R substitution, was cloned into pGEX-6P-1 (GE Healthcare Life Sciences) downstream of GST. The G249R substitution was introduced by a two-step overlapping PCR approach. Following IPTG-mediated (100 μM) induction of protein expression, BL21 cells were lysed by sonication, and incubated with glutathione-sepharose beads. After capture of GST-BAFF on glutathione beads, soluble BAFF was eluted by cleavage of beads with PRESCISSION® Protease (GE Healthcare) for overnight at 4° C.

BAFF Cross-Linking and Western

The chemical cross-linking reagent disuccinimidyl suberate (DSS, Sigma) was used to examine the trimeric property of BAFF as previously described (Douni et al., 2012). Various amounts of BAFF proteins in PBS buffer (pH 7.5) were mixed with 1 mM DSS (the molar ratio of DSS is 10:1). The cross-linking reactions were carried out for 1 hour at room temperature and terminated with 50 mM Tris (pH 7.5) for 30 minutes. Proteins from reaction mixtures were separated on 12% SDS-PAGE and proceeded in Western blot using a polyclonal anti-BAFF antibody (PeproTech).

BAFF/BAFF Receptor ELISA

Nunc plates were coated with 3 μg/ml recombinant soluble human BAFF or BAFF^{G249R} and incubated with increasing amount of recombinant human BAFFR-Fc (R&D Systems). BAFFR binding was detected with a horseradish peroxidase (HRP) conjugated goat anti-human IgG (Fc) (SouthernBiotech, Birmingham, USA) using o-phenylenediamine (OPD) substrate (Thermo Scientific Pierce) that was measured at 490 nm.

MTT Viability Assay

Bone marrow (BM) cells were plated in 96-well plates at a density of 10⁵ cells per well after gradient purification using FICOLL-PAQUE™ (GE Healthcare). BM cells were cultured in αMEM medium (GIBCO) containing 10% fetal bovine serum supplemented with 25 ng/ml M-CSF (R&D Systems) in the presence of the tested compounds at concentrations from 1-20 μM for 2 days (0.1% DMSO). Serum free a-MEM medium containing 0.5 mg/ml MTT [3-(4,5-Dimethylthiazol-2-yl)-2,5-diphenyltetrazolium bromide] was added for 2 hours in a 37° C. CO₂ incubator. After removal of the MTT solution, DMSO was added to extract the dye from the cells and cell viability was accessed at 550 nm.

### REFERENCES

1. Karsenty G. and E. F. Wagner (2002). Reaching a genetic and molecular understanding of skeletal development. Dev. Cell 2:389-406.
2. Boyle W. J., W. S. Simonet, and D. L. Lacey (2003). Osteoclast differentiation and activation. Nature 423:337-342.
3. Fuller K., B. Wong, S. Fox, Y. Choi, T. J. Chambers (1998). TRANCE is necessary and sufficient for osteoblast-mediated activation of bone resorption in osteoclasts. J. Exp. Med. 188:997-1001.
4. Anderson D. M., E. Maraskovsky, W. L. Billingsley, W. C. Dougall, M. E. Tometsko, et al. (1997). A homologue of the TNF receptor and its ligand enhance T-cell growth and dendritic-cell function. Nature 390:175-179.
5. Wong B. R., J. Rho, J. Arron, E. Robinson, J. Orlinick, et al. (1997). TRANCE is a novel ligand of the tumor necrosis factor receptor family that activates c-Jun N-terminal kinase in T cells. J. Biol. Chem. 272:25190-25194.
6. Lam J., C. A. Nelson, F. P. Ross, S. L. Teitelbaum, and D. H. Fremont (2001). Crystal structure of the TRANCE/RANKL cytokine reveals determinants of receptor-ligand specificity. J. Clin. Invest. 108:971-979.
7. Ito S., K. Wakabayashi, O. Ubukata, S. Hayashi, F. Okada, et al. (2002). Crystal structure of the extracellular domain of mouse RANK ligand at 2.2-A resolution. J. Biol. Chem. 277:6631-6636.
8. Hikita A., I. Yana, H. Wakeyama, M. Nakamura, Y. Kadono, et al. (2006). Negative regulation of osteoclastogenesis by ectodomain shedding of receptor activator of NF-kappaB ligand. J. Biol. Chem. 281:36846-36855.
9. Ikeda T., M. Kasai, M. Utsuyama, and K. Hirokawa (2001). Determination of three isoforms of the receptor activator of nuclear factor-kappaB ligand and their differential expression in bone and thymus. Endocrinology 142:1419-1426.
10. Lacey D. L., E. Timms, H. L. Tan, M. J. Kelley, C. R. Dunstan, et al. (1998). Osteoprotegerin ligand is a cytokine that regulates osteoclast differentiation and activation. Cell 93:165-176.
11. Yasuda H., N. Shima, N. Nakagawa, K. Yamaguchi, M. Kinosaki et al. (1998). Osteoclast differentiation factor is a ligand for osteoprotegerin/osteoclastogenesis-inhibitory factor and is identical to TRANCE/RANKL. Proc. Natl. Acad. Sci. U.S.A. 95:3597-3602.
12. Lacey D. L., H. L. Tan, J. Lu, S. Kaufman, G. Van, et al. (2000). Osteoprotegerin ligand modulates murine osteoclast survival in vitro and in vivo. Am. J. Pathol. 157:435-448.
13. Kong Y. Y., H. Yoshida, I. Sarosi, H. L. Tan, E. Timms, et al. (1999). OPGL is a key regulator of osteoclastogenesis, lymphocyte development and lymph-node organogenesis. Nature 397:315-323.
14. Kim N., P. R. Odgren, D. K. Kim, S. C. Marks, Jr., and Y. Choi (2000). Diverse roles of the tumor necrosis factor family member TRANCE in skeletal physiology revealed by TRANCE deficiency and partial rescue by a lymphocyte-expressed TRANCE transgene. Proc. Natl. Acad. Sci. U.S.A. 97:10905-10910.
15. Dougall W. C., M. Glaccum, K. Charrier, K. Rohrbach, K. Brasel, et al. (1999). RANK is essential for osteoclast and lymph node development. Genes Dev. 13:2412-2424.
16. Li J., I. Sarosi, X. Q. Yan, S. Morony, C. Capparelli, et al. (2000). RANK is the intrinsic hematopoietic cell surface receptor that controls osteoclastogenesis and regulation of bone mass and calcium metabolism. Proc. Natl. Acad. Sci. U.S.A. 97:1566-1571.
17. Bucay N., I. Sarosi, C. R. Dunstan, S. Morony, J. Tarpley, et al. (1998). Osteoprotegerin-deficient mice develop early onset osteoporosis and arterial calcification. Genes Dev. 12:1260-1268.
18. Rossi S. W., M. Y. Kim, A. Leibbrandt, S. M. Parnell, W. E. Jenkinson, et al. (2007). RANK signals from CD4(+)3(−) inducer cells regulate development of Aire-expressing epithelial cells in the thymic medulla. J. Exp. Med. 204:1267-1272.
19. Takayanagi H. (2007). Osteoimmunology: shared mechanisms and crosstalk between the immune and bone systems. Nat. Rev. Immunol. 7:292-304.
20. Fata J. E., Y. Y. Kong, J. Li, T. Sasaki, J. Irie-Sasaki, et al. (2000). The osteoclast differentiation factor osteoprotegerin-ligand is essential for mammary gland development. Cell 103 :41-50.
21. Hanada R., A. Leibbrandt, T. Hanada, S. Kitaoka, T. Furuyashiki, et al. (2009). Central control of fever and female body temperature by RANKL/RANK. Nature 462:505-509.
22. Jones D. H., T. Nakashima, O. H. Sanchez, I. Kozieradzki, S. V. Komarova, et al. (2006). Regulation of cancer cell migration and bone metastasis by RANKL. Nature 440:692-696.
23. Schramek D., A. Leibbrandt, V. Sigl, L. Kenner, J. A. Pospisilik, et al. (2010). Osteoclast differentiation factor RANKL controls development of progestin-driven mammary cancer. Nature 468:98-102.
24. Leibbrandt A. and J. M. Penninger (2009). RANK(L) as a key target for controlling bone loss. Adv. Exp. Med. Biol. 647:130-145.
25. Cummings S. R., J. San Martin, M. R. McClung, E. S. Siris, R. Eastell, et al. (2009). Denosumab for prevention of fractures in postmenopausal women with osteoporosis. N. Engl. J. Med. 361:756-765.
26. Smith M. R., B. Egerdie, N. Hernandez Toriz, R. Feldman, T. L. Tammela, et al. (2009). Denosumab in men receiving androgen-deprivation therapy for prostate cancer. N. Engl. J. Med. 361:745-755.
27. Sobacchi C., A. Frattini, M. M. Guerrini, M. Abinun, A. Pangrazio, et al. (2007). Osteoclast-poor human osteopetrosis due to mutations in the gene encoding RANKL. Nat. Genet. 39:960-962.
28. Grigoriadis A. E., Z. Q. Wang, M. G. Cecchini, W. Hofstetter, R. Felix, et al. (1994). c-Fos: a key regulator of osteoclast-macrophage lineage determination and bone remodeling. Science 266:443-448.
29. Yoshida H., S. Hayashi, T. Kunisada, M. Ogawa, S, Nishikawa, et al. (1990). The murine mutation osteopetrosis is in the coding region of the macrophage colony stimulating factor gene. Nature 345:442-444.
30. Suda T., Y. Ueno, K. Fujii, T. Shinki (2003). Vitamin D and bone. J. Cell Biochem. 88:259-266.
31. Xu J., J. W. Tan, L. Huang, X. H. Gao, R. Laird, et al. (2000). Cloning, sequencing, and functional characterization of the rat homologue of receptor activator of NF-kappaB ligand. J. Bone Miner. Res. 15:2178-2186.
32. Cheng T., N. J. Pavlos, C. Wang, J. W. Tan, J. M. Lin, et al. (2009). Mutations within the TNF-like core domain of RANKL impair osteoclast differentiation and activation. Mol. Endocrinol. 23:35-46.
33. Zhang S., C. Liu, P. Huang, S. Zhou, J. Ren, et al. (2009). The affinity of human RANK binding to its ligand RANKL. Arch. Biochem. Biophys. 487:49-53.
34. Zhang X. M., I. Weber, and M. J. Chen (1992). Site-directed mutational analysis of human tumor necrosis factor-alpha receptor binding site and structure-functional relationship. J. Biol. Chem. 267:24069-24075.
35. He M. M., A. S. Smith, J. D. Oslob, W. M. Flanagan, A. C. Braisted, et al. (2005). Small-molecule inhibition of TNF-alpha. Science 310:1022-1025.
36. Liu C., T. S. Walter, P. Huang, S. Zhang, X. Zhu, et al. (2010). Structural and functional insights of RANKL-RANK interaction and signaling. J. Immunol. 184:6910-6919.
37. Aoki K., H. Saito, C. Itzstein, M. Ishiguro, T. Shibata, et al. (2006). A TNF receptor loop peptide mimic blocks RANK ligand-induced signaling, bone resorption, and bone loss. J. Clin. Invest. 116:1525-1534.
38. Ta H. M., G. T. Nguyen, H. M. Jin, J. Choi, H. Park, et al. (2010). Structure-based development of a receptor activator of nuclear factor-kappaB ligand (RANKL) inhibitor peptide and molecular basis for osteopetrosis. Proc. Natl. Acad. Sci. U.S.A. 107:20281-20286.
39. Poblenz A. T., J. J. Jacoby, S. Singh, and B. G. Darnay (2007). Inhibition of RANKL-mediated osteoclast differentiation by selective TRAF6 decoy peptides. Biochem. Biophys. Res. Commun. 359:510-515.
40. Ameloot P., W. Declercq, W. Fiers, P. Vandenabeele, and P. Brouckaert (2001). Heterotrimers formed by tumor necrosis factors of different species or muteins. J. Biol. Chem. 276:27098-27103.
41. Douni E., M. Armaka, D. L. Kontoyiannis, and G. Kollias (2007). Functional genetic and genomic analysis of modeled arthritis. Adv. Exp. Med. Biol. 602:33-42.
42. Justice M. J., D. A. Carpenter, J. Favor, A. Neuhauser-Klaus, M. Hrabe de Angelis, et al. (2000). Effects of ENU dosage on mouse strains. Mamm. Genome 11:484-488.
43. Nelms K. A. and C. C. Goodnow (2001). Genome-wide ENU mutagenesis to reveal immune regulators. Immunity 15:409-418.
44. Broman K. W., H. Wu, S. Sen, and G. A. Churchill (2003). R/qtl: QTL mapping in experimental crosses. Bioinformatics 19:889-890.
45. Sali A. and T. L. Blundell (1993). Comparative protein modelling by satisfaction of spatial restraints. J. Mol. Biol. 234:779-815.
46. Brooks B. R., C. L. Brooks 3rd, A. D. Mackerell Jr., L. Nilsson, R. J. Petrella, et al. (2009). CHARMM: the biomolecular simulation program. J. Comput. Chem. 30:1545-1614.
47. Douni E., V. Rinotas, E. Makrinou, J. Zwerina, J. M. Penninger, E. Eliopoulos, G. Schett, and G. Kollias (2012). A RANKL G278R mutation causing osteopetrosis identifies a functional amino acid essential for trimer assembly in RANKL and TNF. Hum. Mol. Genet. 21(4):784-98.

1. A method of inhibiting trimerization of a TNF superfamily member peptide, the method comprising:
contacting the peptide with a trimerization inhibitor selected from the group consisting of
a) a compound that binds to the TNF superfamily member peptide in the F beta-strand of the peptide provided that when the trimerization inhibitor is 6,7-Dimethyl-3-[[methyl[2-[methyl[[1-[3-(trifluoromethyl)phenyl]-1H-indol-3-yl]methyl]amino]ethyl]amino]methyl]-(4H-1-Benzopyran-4-one), the TNF superfamily member peptide is not TNF-alpha, and
b) a dominant negative TNF superfamily member peptide or fragment thereof.

2. The method according to claim 1, wherein the compound is selected from the group consisting of PRA224, PRA828, PRA123, PRA333, PRA738, and T23.

3. A method for inhibiting osteoclast formation or decreasing bone loss in a subject in need thereof, the method comprising:
administering to the subject an amount of a compound effective to inhibit trimerization of RANKL, wherein the compound is selected from the group consisting of:
a) a compound that binds to the TNF superfamily member peptide in the F beta-strand of the peptide and
b) a dominant negative RANKL peptide or fragment thereof.

4. A method for preventing, treating, or reducing symptoms in a subject diagnosed as being afflicted with osteoporosis, rheumatoid arthritis, multiple myeloma, bone metastasis, juvenile osteoporosis, osteogenesis imperfecta, hypercalcemia, hyperparathyroidism, osteomalacia, osteohalisteresis, osteolytic bone disease, osteonecrosis, Paget's disease of bone, bone loss due to rheumatoid arthritis, inflammatory arthritis, osteomyelitis, periodontal bone loss, bone loss due to cancer, age-related loss of bone mass, osteopenia, and/or inflammatory bowel syndrome, the method comprising:
administering to the subject an amount of a compound effective to inhibit trimerization of RANKL, the compound selected from the group consisting of
a) a compound that binds to the TNF superfamily member peptide in the F beta-strand of the peptide, and
b) a dominant negative RANKL peptide or fragment thereof.

5. The method according to claim 3 or claim 4, wherein the RANKL peptide or fragment thereof comprises a mutation in the F beta-strand at the glycine residue that corresponds to position 279 in human RANKL.

6. The method according to claim 1, claim 3, or claim 4, wherein the compound that binds to the TNF superfamily member peptide is a compound of formula 1, or a stereoisomer thereof, tautomer thereof, or mixture thereof in any ratio; a pharmaceutically acceptable salt, pharmaceutically acceptable solvate, or pharmaceutically acceptable polymorph thereof; 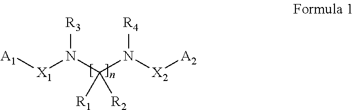
wherein:
A₁ and A₂ are independently a substituted or unsubstituted heterocyclic system selected from: 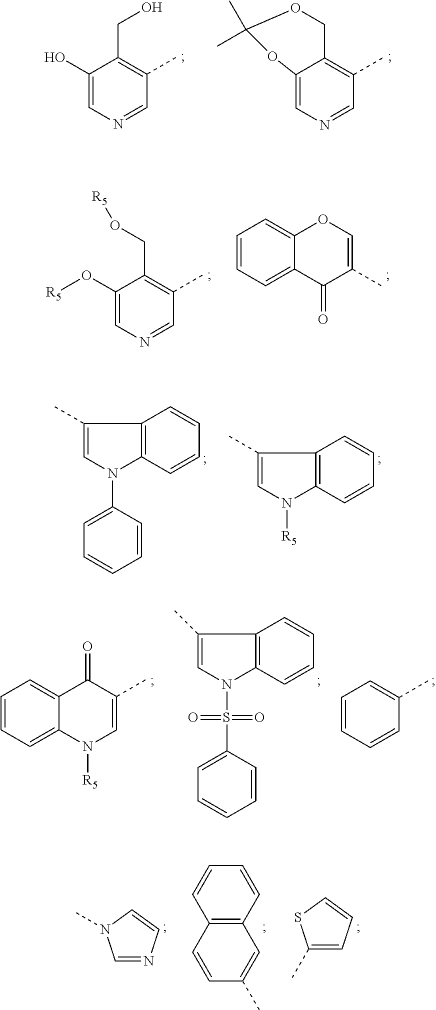
wherein the dotted line(s) indicate(s) the point of attachment, R₅ is hydrogen or (C₁-C₄)-alkyl group and the rings of the heterocyclic systems herein above are unsubstituted or substituted with one or more groups selected from (C₁-C₄)-alkyl, (C₁-C₄)-alkoxy, hydroxyl, hydroxy-(C₁-C₄)-alkyl, fluoroalkyl, halide; nitro (NO₂) and amino (NH₂);
X₁ and X₂ are independently a carbonyl group or a methylene (—CH₂—) group; n is an integer from 2-4;
R₁ and R₂ are independently, hydrogen or (C₁-C₄)-alkyl group;
R₃ and R₄ are independently, hydrogen or (C₁-C₄)-alkyl group;
or wherein R₃ and R₄ are a single (C₁-C₈)-hydrocarbon group connecting the two nitrogen atoms of formula 1, which group may be selected from saturated hydrocarbon (e.g. C₂H₄) and aromatic hydrocarbon.

7. The method according to claim 6, wherein the compound is 6,7-Dimethyl-3-[[methyl[2-[methyl[[1-[3-(trifluoromethyl)phenyl]-1H-indol-3-yl]methyl]amino]ethyl]amino]methyl]-(4H-1-Benzopyran-4-one).

8. The method according to claim 1, wherein the compound is as depicted in FIG. 23.

9. A compound selected from the group consisting of PRA224, PRA828, PRA123, PRA333, and PRA738.

10. A compound having formula 1 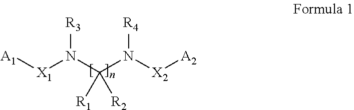
wherein:
A₁ and A₂ are independently a substituted or unsubstituted heterocyclic system selected from: 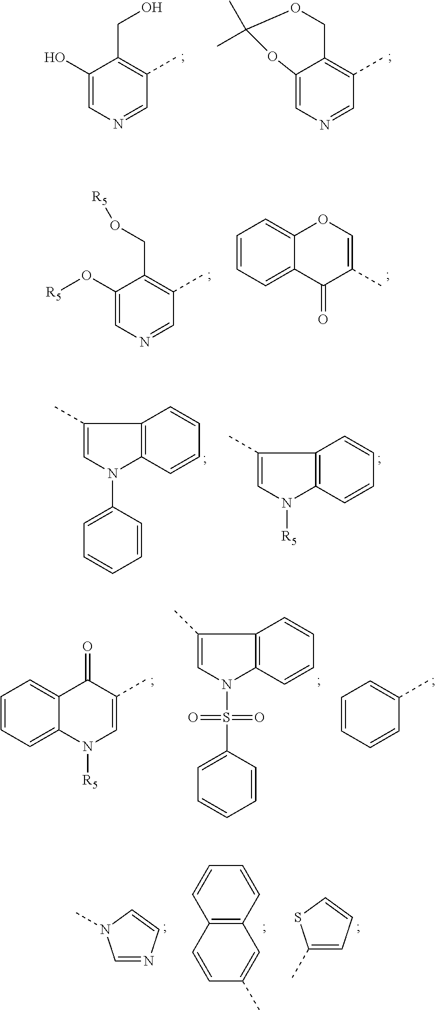
wherein the dotted line(s) indicate(s) the point of attachment, R₅ is hydrogen or (C₁-C₄)-alkyl group and the rings of the heterocyclic systems herein above are unsubstituted or substituted with one or more groups selected from (C₁-C₄)-alkyl, (C₁-C₄)-alkoxy, hydroxyl, hydroxy-(C₁-C₄)-alkyl, fluoroalkyl, halide; nitro (NO₂) and amino (NH₂);
X₁ and X₂ are independently a carbonyl group or a methylene (—CH₂—) group; n is an integer from 2-4;
R₁ and R₂ are independently, hydrogen or (C₁-C₄)-alkyl group;
R₃ and R₄ are independently, hydrogen or (C₁-C₄)-alkyl group;
or wherein R₃ and R₄ are a single (C₁-C₈)-hydrocarbon group connecting the two nitrogen atoms of formula 1, which group may be selected from saturated hydrocarbon and aromatic hydrocarbon;
with the proviso that when A₁ and A₂ are both selected from: 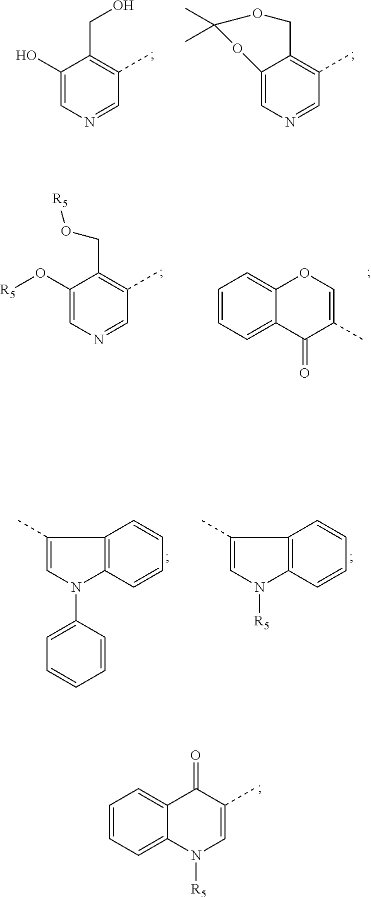
then at least one of the heterocyclic systems is substituted with one or more groups selected from halide; nitro (NO₂) and amino (NH₂).

11. The compound of claim 10, wherein the heterocyclic systems are unsubstituted or substituted with one or more groups selected from the group consisting of trifluoromethyl (CF₃), fluoro (F), nitro (NO₂), and amino (NH₂).

12. A TNF superfamily member peptide or fragment thereof that inhibits trimerization of the TNF superfamily member having a dominant negative mutation in the trimerization domain.

13. The TNF superfamily member peptide or functional fragment thereof of claim 12, comprising a mutation in F beta-strand.

14. The TNF superfamily member peptide or a functional fragment thereof of claim 13, the TNF superfamily member peptide comprising peptide having at least 80% sequence identity to K L E A Q P F A H L T I N A T D I P S G S H K V S L S S W Y H D R G W A K I S N M T F S N G K L I V N Q D G F Y Y L Y A N I C F R H H E T S G D L A T E Y L Q L M V Y V T K T S I K I P S S H T L M K G G S T K Y W S G N S E F H F Y S I N V G X F F K L R S G E E I S I E V S N P S L L D P D Q D A T Y F G A F K V R D I D (SEQ ID NO:3), wherein X is not glycine.

15. An isolated polynucleotide encoding the TNF superfamily member peptide or fragment thereof of claim 12.

16. A non-human animal comprising the polynucleotide of claim 15.

17. A vector comprising the polynucleotide of claim 15.

18. A cell comprising the vector of claim 17.

19. A pharmaceutical composition comprising:
the TNF superfamily member peptide or fragment thereof of claim 12, and
a pharmaceutically acceptable carrier.

20. A liposome comprising the TNF superfamily member peptide or fragment thereof of claim 9.
